(19) United States
(12) Patent Application Publication
(10) Pub. No.: US 2025/0295756 A1
(43) Pub. Date: Sep. 25, 2025

(54) LIVE ATTENUATED SARS-COV-2 AND A VACCINE MADE THEREOF

(71) Applicant: FREIE UNIVERSITÄT BERLIN, Berlin (DE)

(72) Inventors: Jakob TRIMPERT, Berlin (DE); Dusan KUNEC, Berlin (DE); Nikolaus OSTERRIEDER, Potsdam (DE)

(73) Assignee: FREIE UNIVERSITÄT BERLIN, Berlin (DE)

(21) Appl. No.: 18/730,516

(22) PCT Filed: Jan. 20, 2022
(86) PCT No.: PCT/EP2022/051215

§ 371 Date: Jul. 19, 2024

(51) Int. Cl. A61K 39/215; A61K 39/00; A61P 37/04; C12N 7/00

(52) U.S. Cl. CPC A61K 39/215; A61P 37/04; C12N 7/00; A61K 2039/5254; A61K 2039/542; A61K 2039/543; A61K 2039/545; C12N 2770/20021; C12N 2770/20022; C12N 2770/20034

(57) ABSTRACT

It is provided a polynucleotide encoding a) severe acute respiratory syndrome coronavirus 2 (SARS-CoV-2) spike protein; and/or b) at least one non-structural SARS-CoV-2 protein selected from the group consisting of non-structural protein 7, non-structural protein 8, non-structural protein 9, non-structural protein 10, non-structural protein 11, non-structural protein 12, an endoribonuclease, and a 2′-O-methyltransferase, wherein the polynucleotide comprises or consists of at least one sequence part comprising codon-pair deoptimizations in comparison to the SARS-CoV-2 genome.

### CROSS-REFERENCE TO A RELATED APPLICATION

This application is the United States national phase of International Patent Application No. PCT/EP2022/051215, filed on Jan. 20, 2022, the disclosure of which is hereby incorporated by reference in its entirety.

### TECHNICAL FIELD

The disclosure relates to a codon-pair deoptimized polynucleotide encoding a respiratory syndrome coronavirus 2 (SARS-CoV-2) protein, to a live attenuated SARS-CoV-2 comprising such polynucleotide, to a pharmaceutical composition comprising such a live attenuated SARS-CoV-2, to a vaccination method for administering this pharmaceutical composition, to a vector comprising such polynucleotide, to a host cell comprising such polynucleotide, as well as to method of producing a virus.

### BACKGROUND

Severe acute respiratory syndrome coronavirus 2 (SARS-CoV-2) emerged in December 2019 as the causative agent of coronavirus disease 2019 (COVID-19) (Wu et al., 2020; Zhou et al., 2020b). The virus is highly transmissible among humans (Chan et al., 2020). It has spread rapidly around the world within a matter of weeks and the world is still battling with the ongoing COVID-19 pandemic.

SARS-CoV-2 primarily replicates in the upper respiratory tract (Zou et al., 2020). The infection with SARS-CoV-2 can cause a wide spectrum of clinical manifestations, ranging from asymptomatic to life-threatening disease conditions (Chen et al., 2020; Zhou et al., 2020a). Especially the elderly and patients with pre-existing conditions are at greater risk of developing more severe disease such as pneumonia, acute respiratory distress syndrome and multiple organ failure (Chen et al., 2020; Garg et al., 2020; Zhou et al., 2020a). The ongoing pandemic imposes an enormous health, psychological, economic, and social burden. To date (December 2021) more than 270 million people have been infected with the virus, of whom more than 5.3 million have died as a result of the infection (https://coronavirus.jhu.edu/map.html) (Dong et al., 2020).

The unprecedented scale and severity of the COVID-19 pandemic prompted the rapid development of novel diagnostic tests, therapeutics and vaccines which could be used to contain the spread of the virus and limit the pandemic. Globally, more than 90 vaccines are being tested in clinical trials, but only few have reached the final stages of testing (Zimmer et al., 2021). Almost all vaccines that have been or are being evaluated in clinical trials are based either on inactivated or subunit virus preparations (Ella et al., 2021; Gao et al., 2020; Wang et al., 2020; Zhang et al., 2021), replication-defective virus vectors (Emary et al., 2021; Logunov et al., 2021; Solforosi et al., 2021; Voysey et al., 2021b; Zhu et al., 2020), or DNA/RNA molecules (Anderson et al., 2020; Baden et al., 2021; Corbett et al., 2020; Dagan et al., 2021; Jackson et al., 2020; Mulligan et al., 2020; Polack et al., 2020; Sahin et al., 2020; Walsh et al., 2020).

SARS-CoV-2, positioned at the opposite end of the incipient arms race, is rapidly evolving (Tegally et al., 2021; Faria et al., 2021; Davies et al., 2021). Benefiting from its global presence, the virus continues to adapt to its new host and to infection- or vaccine-induced immunity. During the course of the pandemic, a number of genetic variants have emerged (Tegally et al., 2021; Faria et al., 2021; Davies et al., 2021). Variants that exhibit increased infectivity, cause greater morbidity and mortality, or have the ability to evade infection- or vaccine-induced immunity pose an increased threat to public health. The World Health Organization (WHO) and other national health agencies have independently established classification systems that categorize emerging variants as variants of interest (VOIs), variants under investigation (VUIs), or variants of concern (VOCs) based on their risk to public health (cf. Table 1 of Trimpert et al. “Live attenuated virus vaccine protects against SARS-CoV-2 variants of concern B.1.1.7 (Alpha) and B.1.351 (Beta)”, Science Advances, Vol. 7, No. 49 (2021)). In addition, to simplify communication with the public, the WHO recommends that VOIs and VOCs should also be labeled using the letters of the Greek alphabet. As of 12 Aug. 2021, viruses belonging to lineages B.1.1.7 (Alpha), B.1.351 (Beta), B.1.1.28.1 (Gamma), B.1.617.2 (Delta), and, most recently, B.1.159.1 (Omicron) are classified by several health agencies as VOCs. In countries where they emerged, these variants rapidly supplanted the preexisting variants and started to spread globally.

The B.1.1.7 variant, first detected in the United Kingdom in December 2020, is 50 to 100% more transmissible and possibly also more lethal than earlier variants but shows no tendency to evade immunity induced by infection or vaccination (Davies et al., 2021; Volz et al., 2021; Abu-Raddad et al., 2021). The B.1.1.7 variant has been detected in 132 countries and rapidly became the dominant variant in Europe and the United States. The B.1.351 variant, first detected in South Africa in May 2020, is not only more transmissible but also capable of reinfecting individuals and of breaking through vaccine protection (Madhi et al., 2021; Johnson & Johnson; Naveca et al., 2021). The B.1.1.28.1 variant, better known as P.1, is similar to B.1.351 in that both share some important mutations in the spike glycoprotein (E484K, K417N/T, and N501Y). B.1.1.28.1 emerged in late 2020 in Manaus, Brazil (Faria et al., 2021). Similar to the B.1.351 variant, it can cause reinfection because it can bypass immunity developed after infection with other virus variants (Faria et al., 2021; Naveca et al., 2021). It is estimated that B.1.1.28.1 is 40 to 140% more transmissible, more pathogenic, and 10 to 80% more lethal than other variants (Faria et al., 2021). Most recently, on 7 May 2021, the WHO reclassified the B.1.617.2 variant, first detected in India, as a VOC due to its high transmissibility (WHO). As of August 2021, B.1.617.2 has largely outcompeted B.1.1.7 and is now the predominant variant in Europe and the United States. According to the WHO, B.1.617.2 is the most dangerous strain worldwide, and it has attracted considerable attention for its ability to evade infection- and vaccine-mediated protection (Dyer et al., 2021).

### SUMMARY

It is an object underlying the proposed solution to provide novel SARS-CoV-2 vaccine candidates.

This object is achieved with a specific polynucleotide encoding a) severe acute respiratory syndrome coronavirus 2 (SARS-CoV-2) spike protein; and/or b) at least one non-structural SARS-CoV-2 protein selected from the group consisting of non-structural protein 7, non-structural protein 8, non-structural protein 9, non-structural protein 10, non-structural protein 11, non-structural protein 12, an endoribonuclease (also referred to as non-structural protein 15), and a 2′-O-methyltransferase (also referred to as non-structural protein 16). In this context, the polynucleotide comprises or consists of at least one sequence part comprising codon-pair deoptimizations in comparison to the corresponding SARS-CoV-2 genome part.

The term “polynucleotide”, as used herein, refers to a molecule containing multiple nucleotides (e.g. mRNA, RNA, CRNA, cDNA or DNA). The term typically refers to oligonucleotides greater than 200, preferably greater than 300, preferably greater than 400, preferably greater than 500, preferably greater than 600, preferably greater than 700, preferably greater than 800, preferably greater than 900, preferably greater than 998 nucleotide residues in length. The polynucleotide of the proposed solution either essentially consists of the nucleic acid sequences described herein or comprises the aforementioned nucleic acid sequences. Thus, it may contain further nucleic acid sequences as well. The term polynucleotide encompasses single stranded as well as double stranded polynucleotides. Moreover, encompassed herein are also modified polynucleotides including chemically modified polynucleotides, artificial modified polynucleotides, or naturally occurring modified polynucleotides such as glycosylated or methylated polynucleotides.

The term “SARS-CoV-2” or “severe acute respiratory syndrome coronavirus 2”, as used herein, refers to any variant that is classified as SARS-CoV-2. In some embodiments, the SARS-CoV-2 described herein is at least one SARS-CoV-2 variant selected from the group consisting of Alpha, Beta, Gamma, Delta or Omicron variant. In some embodiments, the SARS-CoV-2 refers to a SARS-CoV-2 variant comprising a mutation selected from the group consisting of del 69-70, RSYLTPGD246-253N, N440K, G446V, L452R, Y453F, S477G/N, E484Q, E484K, F490S, N501Y, N501S, D614G, Q677P/H, P681H, P681R, and A701V. Therefore, “SARS-CoV-2 protein” and “SARS-CoV-2 genome” may also be understood as the protein and genome of a SARS-CoV-2 variant, respectively.

The term “codon”, as used herein, refers to any group of three consecutive nucleotides in a coding part of a polynucleotide such as a messenger RNA molecule, or coding strand of DNA that specifies a particular amino acid, a starting or stopping signal for translation. Typically, codons are specific for one amino acid, however cases of a codon sharing at least one nucleotide with another codon are known for SARS-CoV-2.

The term “codon pair”, as used herein refers to two consecutive codons.

The term “codon-pair deoptimization”, as used herein, refers to recoding of codons such that the encoded protein is the same, but suboptimal codon pairs and/or CpG dinucleotides emerge. Methods for codon-pair deoptimizations are known in the art (see, e.g., Coleman et al., 2008, Mueller et al., 2010). In some embodiments, the codon-pair deoptimization described herein comprises increasing the number of suboptimal codon pairs and CpG dinucleotides in recoded genomes. In some embodiments, the codon-pair deoptimization(s) described herein result(s) in increased mRNA decay and/or reduced translation efficiency. In some embodiments, the codon-pair deoptimization(s) described herein result(s) in less protein, less virus, reduced virulence, and/or a live-attenuated virus.

The polynucleotide of the solution may be used in virus production or in the context of a vaccine. As such, the polynucleotide of the solution emits a decreased risk of uncontrolled replication during production, transport, storage, processing and/or administration compared to the wild type sequence.

Typically, this polynucleotide forms part of a live attenuated SARS-CoV-2. In comparison to a wild type virus, a live attenuated virus provokes less and/or less severe or even no symptoms in a host organism after the host organism has been confronted (infected) with the attenuated virus. At the same time, the live attenuated virus induces an immune response of the host to the attenuated virus that is at least partially protective against a wild type virus infection and/or at least one symptom thereof.

In contrast to most of the vaccines under development, the inventors generated attenuated but replicating SARS-CoV-2 vaccine candidates by genetic modification of the SARS-CoV-2 genome via codon pair deoptimization (CPD) (Coleman et al., 2008). CPD is a virus attenuation strategy which has enabled rapid and highly efficient attenuation of a wide variety of viruses (Broadbent et al., 2016; Coleman et al., 2008; Eschke et al., 2018; Groenke et al., 2020; Khedkar et al., 2018; Kunec and Osterrieder, 2016; Le Nouen et al., 2014; Mueller et al., 2010; Shen et al., 2015; Trimpert et al., 2021a; Trimpert et al., 2021b). CPD rearranges the positions of existing synonymous codons in one or more viral genes, without changing the codon bias or amino acid composition of the encoded protein (Eschke et al., 2018; Groenke et al., 2020; Khedkar et al., 2018; Kunec and Osterrieder, 2016; Osterrieder and Kunec, 2018; Trimpert et al., 2021a; Trimpert et al., 2021b). Naturally underrepresented codons can become overrepresented by CPD. Since the effect of CPD strongly depends on the genome sequence to be deoptimized, no general measure of codons to be deoptimized can be indicated. However, the person skilled in the art is aware how to identify or estimate codon pairs that can be replaced by naturally underrepresented codon pairs at the target site (e.g. in a target species or a target tissue) at which the polynucleotide is intended to be translated. The inventors provide herein examples which codon pairs to recode to achieve overrepresentation of naturally underrepresented codon pairs and codon-pair deoptimization of the polynucleotide. The skilled person could therefore—at least from the codon pairs at the target site and the means and methods provided herein—arrive at other polynucleotides according to the solution.

A polynucleotide is to be considered as codon-pair deoptimized if at least one codon pair is deoptimized with respect to the corresponding natural sequence. Recoded viruses typically do not produce proteins from the recoded genes as efficiently as their parents, and show defects in reproductive fitness, which enables the host to control wild-type virus infection by innate and adaptive immune responses (Eschke et al., 2018; Groenke et al., 2020; Khedkar et al., 2018; Kunec and Osterrieder, 2016; Mueller et al., 2010; Trimpert et al., 2021b; Wimmer et al., 2009). The conserved antigenic identity and replicative potential enable recoded attenuated viruses to fully engage the immune system of the host and provoke strong immune responses.

Thus, by the codon-pair deoptimization, the resulting proteins are not altered. Rather, even though the genomic sequence of SARS-CoV-2 is altered, the resulting proteins remain the same. However, typically the efficiency of translation is reduced so that the virus replication is also reduced. This leads to an immune response when the live attenuated SARS-CoV-2 is used as vaccine without the risk of a pathologic virus replication in a patient having received the vaccine.

Another possible effect of codon-pair deoptimization is a CpG mediated immune response leading to virus attenuation. The present disclosure is not limited to a specific one of these effects.

In an embodiment, the polynucleotide encodes the non-structural protein 7. In an embodiment, the polynucleotide encodes the non-structural protein 8. In an embodiment, the polynucleotide encodes the non-structural protein 9. In an embodiment, the polynucleotide encodes the non-structural protein 10. In an embodiment, the polynucleotide encodes the non-structural protein 11. In an embodiment, the polynucleotide encodes the non-structural protein 12. In an embodiment, the polynucleotide encodes the endonuclease. In an embodiment, the polynucleotide encodes the 2′-O-methyltransferase. In an embodiment, the polynucleotide encodes the spike protein (sometimes also referred to as spike glycoprotein).

In an embodiment, the polynucleotide encodes at least two of the non-structural proteins. To give an example, the polynucleotide encodes, in an embodiment, the endoribonuclease and the 2′-O-methyltransferase. To give another example, the polynucleotide encodes, in an embodiment, non-structural protein 7, non-structural protein 8, non-structural protein 9, non-structural protein 10, and non-structural protein 11.

In an embodiment, the SARS-CoV-2 genome is a genome section extending from position 11,000 to position 27,000 of the genome of SARS-CoV-2. For position numbering and definition of the terms “genome of SARS-CoV-2” and “wild type SARS-CoV-2”, reference is made to the gene bank accession number MT108784.1 (freely accessible via the website https://www.ncbi.nlm.nih.gov/genbank/) that comprises 29,891 bases or nucleotides. The first of these bases or nucleotides (at the 5′ terminus) is positioned at position 1. The last of these bases or nucleotides (at the 3′ terminus) is positioned at position 29,891. The skilled person is aware of how to adjust the numbering of the referenced sequence to embodiments, wherein the SARS-CoV-2 genome understood as a sequence from a different SARS-CoV-2 variant. In some embodiments, the polynucleotide of the solution is a codon-pair deoptimized sequence of a sequence comprised in the SARS COV-2 genome section from position 11,000 to position 24,000. In an embodiment, the genome section extends from position 11,500 to position 26,000, in particular from position 11,900 to position 25,500, in particular from position 11,950 to position 25,350, in particular from position 12,000 to position 24,000. In an embodiment, the genome section extends from position 11,950 to position 14,400. In an embodiment, the genome section extends from position 11,900 to position 13,500. In an embodiment, the genome section extends from position 13,900 to position 14,400. In an embodiment, the genome section extends from position 20,300 to position 21,600. In an embodiment, the genome section extends from position 24,300 to position 25,400. These embodiments can be combined in any desired way.

In an embodiment, the at least one sequence part comprising codon-pair deoptimizations has a length lying in a range of from 750 nucleotides to 2500 nucleotides, in particular of from 800 nucleotides to 2400 nucleotides, in particular of from 900 nucleotides to 2300 nucleotides, in particular of from 999 nucleotides to 2200 nucleotides, in particular of from 1000 nucleotides to 2100 nucleotides, in particular of from 1100 nucleotides to 2000 nucleotides, in particular of from 1146 nucleotides to 1900 nucleotides, in particular of from 1200 nucleotides to 1836 nucleotides, in particular of from 1300 nucleotides to 1800 nucleotides, in particular of from 1400 nucleotides to 1700 nucleotides, in particular of from 1500 nucleotides to 1600 nucleotides.

In an embodiment, between 15% and 40%, in particular between 20% and 35%, in particular between 25% and 30% of the nucleotides of the at least one sequence part comprising codon-pair deoptimizations are different from the nucleotides of a corresponding (wild type) SARS-CoV-2 genome. Such a wild-type SARS-CoV-2 genome is the genomic sequence of a non-artificially modified virus variant or lineage, such as lineages B.1.1.7 (Alpha), B.1.351 (Beta), B.1.1.28.1 (Gamma), B.1.617.2 (Delta), or B.1.159.1 (Omicron). It can also be denoted as authentic SARS-CoV-2 genome or authentic SARS-CoV-2 genomic sequence.

In an embodiment, between 200 and 500 nucleotides, in particular between 250 and 450 nucleotides, in particular between 300 and 400 nucleotides of the at least one sequence part comprising codon-pair deoptimizations are different from the (in particular identically positioned) nucleotides of a corresponding SARS-CoV-2 genome.

In an embodiment, between 40% and 70%, in particular between 45% and 65%, in particular between 50% and 60%, in particular between 55% and 62% of the codons of the at least one sequence part comprising codon-pair deoptimizations are different from the respective codons of a corresponding SARS-CoV-2 genome.

In an embodiment, between 150 and 400 codons, in particular between 200 and 350 codons, in particular between 250 and 300 codons of the at least one sequence part comprising codon-pair deoptimizations are different from the (in particular identically positioned) codons of a corresponding SARS-CoV-2 genome.

In an embodiment, the at least one sequence part comprising codon-pair deoptimizations comprises a first deoptimized sequence part and a second deoptimized sequence part. Both deoptimized sequence parts are separated from each other by a non-deoptimized sequence section comprising at least 300 nucleotides, e.g., 300 to 1000 nucleotides, in particular 400 to 900 nucleotides, in particular 500 to 800 nucleotides, in particular 600 to 700 nucleotides. By conserving a specific part of the RNA sequence and by deoptimizing flanking parts upstream and downstream of the conserved RNA sequence, a particularly high efficacy in attenuating the SARS-CoV-2 is achieved while maintaining its ability to replicate.

In an embodiment, the first deoptimized sequence part has a length lying in a range of from 1300 nucleotides to 1600 nucleotides, in particular of from 1400 nucleotides to 1500 nucleotides, in particular of from 1450 nucleotides to 1490 nucleotides. At the same time, the second deoptimized sequence part has a length lying in a range of from 100 nucleotides to 400 nucleotides, in particular of from 200 nucleotides to 300 nucleotides, in particular of from 350 nucleotides to 400 nucleotides. The length of the first deoptimized sequence part and of the second deoptimized sequence part is chosen such that other applicable restrictions (such as an overall length of the at least one sequence part comprising codon-pair deoptimizations of not more than 2000 nucleotides) are fulfilled, if desired. If the length of the at least one sequence part comprising codon-pair deoptimizations shall not exceed 2000 nucleotides, it is immediately apparent that only the lower threshold of 1300 nucleotides can be combined with the upper threshold of 400 nucleotides for the first and second deoptimized sequence parts to fulfil the restriction of the maximum length of the at least one sequence part comprising codon-pair deoptimizations, considering that the first deoptimized sequence part and the second deoptimized sequence part are separated by at least 300 nucleotides of the authentic SARS-CoV-2 genome. At the same time, the upper threshold of 1600 nucleotides for the first deoptimized sequence part can be combined with the lower threshold of 100 nucleotides for the second deoptimized sequence part to fulfil a maximum length of 2000 nucleotides, considering the intermediate 300 non-recoded nucleotides.

In an embodiment, the first deoptimized sequence part is at least 95%, in particular at least 96%, in particular at least 97%, in particular at least 98%, in particular at least 99%, in particular 100% identical to SEQ ID NO. 2. At the same time, the second deoptimized sequence part is at least 95%, in particular at least 96%, in particular at least 97%, in particular at least 98%, in particular at least 99%, in particular 100% identical to SEQ ID NO. 4.

In an embodiment, the polynucleotide is at least 95%, in particular at least 96%, in particular at least 97%, in particular at least 98%, in particular at least 99%, in particular 100% identical to SEQ ID NO. 6.

In an embodiment, the polynucleotide is at least 95%, in particular at least 96%, in particular at least 97%, in particular at least 98%, in particular at least 99%, in particular 100% identical to SEQ ID NO. 8.

In an embodiment, the polynucleotide is at least 95%, in particular at least 96%, in particular at least 97%, in particular at least 98%, in particular at least 99%, in particular 100% identical to SEQ ID NO. 10.

In an embodiment, the polynucleotide is at least 95%, in particular at least 96%, in particular at least 97%, in particular at least 98%, in particular at least 99%, in particular 100% identical to SEQ ID NO. 15.

In an embodiment, the polynucleotide is at least 95%, in particular at least 96%, in particular at least 97%, in particular at least 98%, in particular at least 99%, in particular 100% identical to SEQ ID NO. 16.

In an embodiment, the polynucleotide is at least 95%, in particular at least 96%, in particular at least 97%, in particular at least 98%, in particular at least 99%, in particular 100% identical to SEQ ID NO. 17.

In an aspect, the solution relates to a live attenuated SARS-CoV-2. This live attenuated SARS-CoV-2 comprises a partially recoded genomic RNA sequence, i.e., a partially recoded genomic viral sequence. The partially recoded genomic RNA sequence is a codon-pair deoptimized sequence coding for a spike protein and/or a specific non-structural protein (nsp). The non-structural protein is chosen from the group consisting of non-structural protein 7, non-structural protein 8, non-structural protein 9, non-structural protein 10, non-structural protein 11 (a small, only 13 amino acids long protein), non-structural protein 12 (also referred to as RNA-dependent RNA polymerase), an endoribonuclease, and a 2′-O-methyltransferase of the live attenuated SARS-CoV-2.

In an embodiment, the partially recoded genomic RNA sequence codes for the non-structural protein 12. In an embodiment, the partially recoded genomic sequence codes for the spike protein (sometimes also referred to as spike glycoprotein).

In an embodiment, the partially recoded genomic RNA sequence comprises at least two of the non-structural proteins. To give an example, the partially recoded genomic RNA sequence codes, in an embodiment, for the endoribonuclease and the 2′-O-methyltransferase. To give another example, the partially recoded genomic RNA sequence codes, in an embodiment, for non-structural protein 7, non-structural protein 8, non-structural protein 9, non-structural protein 10, and non-structural protein 11.

In an embodiment, the partially recoded genomic RNA sequence lies in a genome section extending from position 11,000 to position 27,000 of the genome of the live attenuated SARS-CoV-2. According to gene bank accession number MT108784.1, the genome of the wild type SARS-CoV-2 comprises 29,891 bases or nucleotides. The genome of the live attenuated SARS-CoV-2 has essentially a similar length. The only difference is the length of a polyA tail at the 3′ terminus that was determined by sequencing to be eight adenine nucleotides longer than in case of the wild type SARS-CoV-2. It should be noted that some uncertainty remains by determining the length of a polyA tail by sequencing. Consequently, it is possible that the polyA tail in the wild type sequence is longer or shorter than indicated in the sequence according to gene bank accession number MT108784.1. Likewise, it is possible that the polyA tail in the live attenuated SARS-CoV-2 sequence is longer or shorter than presently determined.

The first of the 29,891 bases or nucleotides of the genome of the wild type SARS-CoV-2 (at the 5′ terminus) is positioned at position 1. The last of these bases or nucleotides (at the 3′ terminus) is positioned at position 29,891. In an embodiment, the genome section extends from position 11,500 to position 26,000, in particular from position 11,900 to position 25,500, in particular from position 11,950 to position 25,350, in particular from position 12,000 to position 24,000. In an embodiment, the genome section extends from position 11,950 to position 14,400. In an embodiment, the genome section extends from position 11,900 to position 13,500. In an embodiment, the genome section extends from position 13,900 to position 14,400. In an embodiment, the genome section extends from position 20,300 to position 21,600. In an embodiment, the genome section extends from position 24,300 to position 25,400. These embodiments can be combined in any desired way.

In an embodiment, the partially recoded genomic RNA sequence has a length lying in a range of from 750 nucleotides to 2500 nucleotides, in particular of from 800 nucleotides to 2400 nucleotides, in particular of from 900 nucleotides to 2300 nucleotides, in particular of from 999 nucleotides to 2200 nucleotides, in particular of from 1000 nucleotides to 2100 nucleotides, in particular of from 1100 nucleotides to 2000 nucleotides, in particular of from 1146 nucleotides to 1900 nucleotides, in particular of from 1200 nucleotides to 1836 nucleotides, in particular of from 1300 nucleotides to 1800 nucleotides, in particular of from 1400 nucleotides to 1700 nucleotides, in particular of from 1500 nucleotides to 1600 nucleotides.

In an embodiment, between 15% and 40%, in particular between 20% and 35%, in particular between 25% and 30% of the nucleotides of the partially recoded genomic RNA sequence are different from the nucleotides of a corresponding wild-type genomic RNA sequence. Such a wild-type genomic RNA sequence is the genomic viral sequence of a non-artificially modified virus variant or lineage, such as lineages B.1.1.7 (Alpha), B.1.351 (Beta), B.1.1.28.1 (Gamma), B.1.617.2 (Delta), or B.1.159.1 (Omicron). It can also be denoted as authentic SARS-CoV-2 genomic RNA sequence.

In an embodiment, between 200 and 500 nucleotides, in particular between 250 and 450 nucleotides, in particular between 300 and 400 nucleotides of the partially recoded genomic RNA sequence are different from the identically positioned nucleotides of a corresponding wild-type virus genomic RNA sequence.

In an embodiment, between 40% and 70%, in particular between 45% and 65%, in particular between 50% and 60%, in particular between 55% and 62% of the codons (i.e., three nucleotides in each case that code for a specific amino acid) of the partially recoded genomic RNA sequence are different from the respective codons of a corresponding wild-type virus genomic RNA sequence.

In an embodiment, between 150 and 400 codons, in particular between 200 and 350 codons, in particular between 250 and 300 codons of the partially recoded genomic RNA sequence are different from the identically positioned codons of a corresponding wild-type virus genomic RNA sequence.

In an embodiment, the partially recoded genomic RNA sequence comprises a first recoded part and a second recoded part. Both recoded parts are separated from each other by a non-recoded genome section comprising at least 300 nucleotides, e.g., 300 to 1000 nucleotides, in particular 400 to 900 nucleotides, in particular 500 to 800 nucleotides, in particular 600 to 700 nucleotides. By conserving a specific part of the RNA sequence and by recoding flanking parts upstream and downstream of the conserved RNA sequence, a particularly high efficacy in attenuating the SARS-CoV-2 while maintaining its general viability is achieved.

In an embodiment, the first recoded part has a length lying in a range of from 1300 nucleotides to 1600 nucleotides, in particular of from 1400 nucleotides to 1500 nucleotides, in particular of from 1450 nucleotides to 1490 nucleotides. At the same time, the second recoded part has a length lying in a range of from 100 nucleotides to 400 nucleotides, in particular of from 200 nucleotides to 300 nucleotides, in particular of from 350 nucleotides to 400 nucleotides. The length of the first recoded part and of the second recoded part is chosen such that other applicable restrictions (such as an overall length of the recoded genomic RNA sequence of not more than 2000 nucleotides) are fulfilled, if desired. If the length of the partially recoded genomic RNA sequence shall not exceed 2000 nucleotides, it is immediately apparent that only the lower threshold of 1300 nucleotides can be combined with the upper threshold of 400 nucleotides for the first and second recoded parts to fulfil the restriction of the maximum length of the partially recoded genomic RNA sequence, considering that the first recoded part and the second recoded part are separated by at least 300 nucleotides of the authentic SARS-CoV-2 genomic sequence. At the same time, the upper threshold of 1600 nucleotides for the first recoded part can be combined with the lower threshold of 100 nucleotides for the second recoded part to fulfil a maximum length of 2000 nucleotides, considering the intermediate 300 non-recoded nucleotides.

In an embodiment, the first recoded part is at least 95%, in particular at least 96%, in particular at least 97%, in particular at least 98%, in particular at least 99%, in particular 100% identical to SEQ ID NO. 2. At the same time, the second recoded part is at least 95%, in particular at least 96%, in particular at least 97%, in particular at least 98%, in particular at least 99%, in particular 100% identical to SEQ ID NO. 4.

In an embodiment, the partially recoded genomic RNA sequence is at least 95%, in particular at least 96%, in particular at least 97%, in particular at least 98%, in particular at least 99%, in particular 100% identical to SEQ ID NO. 6.

In an embodiment, the partially recoded genomic RNA sequence is at least 95%, in particular at least 96%, in particular at least 97%, in particular at least 98%, in particular at least 99%, in particular 100% identical to SEQ ID NO. 8.

In an embodiment, the partially recoded genomic RNA sequence is at least 95%, in particular at least 96%, in particular at least 97%, in particular at least 98%, in particular at least 99%, in particular 100% identical to SEQ ID NO. 10.

The phrase “being % identical” or “having percent (%) sequence identity” with respect to a reference sequence is defined as the percentage of nucleotides or amino acid residues in a candidate sequence that are identical with the nucleotides or amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The skilled person is aware that sequences as exemplified herein can be altered to a certain percentage without or without substantially altering biologic functions such as attenuation or the encoded protein. Therefore, the skilled person using means and methods described herein can amend codon pairs according to the teaching of the solution. E.g., codon pairs can be replaced with synonymous versions that are similarly attenuating and/or naturally underrepresented as the deoptimized codon pairs of the sequences described herein. Thus, a similar degree of deoptimization may be achieved by replacing codon pairs in the position ranges described herein.

The solution is based at least in part on the finding that codon deoptimizations in the position ranges described herein are particularly useful for attenuating the SARS-CoV-2 virus while sufficiently maintaining immunogenicity and the ability of the virus to replicate.

The individual recoded sequences and their position in the SARS-CoV-2 genome are summarized in the following Table 1.

TABLE 1
Summary of recoded RNA sequences.

| | | | | | | Position in | |
|---|---|---|---|---|---|---|---|
| | | | | | | SARS-CoV-2 | |
| | SEQ | | | | | genome | |
| Sequence | ID | | Recoded | Length | Length | (NC_045512.2) | |
| name | NO. | Description | protein | (nt) | (aa) | Start | End |
| WT6A | 1 | first part of original | pp1a/pp1ab - | 1.482 | 494 | 11.969 | 13.450 |
| | | sequence, to be | nsp7, nsp8, | | | | |
| | | replaced by first | nsp9, nsp10, | | | | |
| | | part of CPD sequence | nsp11 and nsp12 | | | | |
| CPD6A | 2 | first part of CPD | Pp1a/pp1ab - | 1.482 | 494 | 11.969 | 13.450 |
| | | sequence | nsp7, nsp8, | | | | |
| | | | nsp9,nsp10, | | | | |
| | | | nsp11 and nsp12 | | | | |
| WT6B | 3 | second part of | pp1ab - nsp12 | 354 | 118 | 13.954 | 14.307 |
| | | original sequence, | | | | | |
| | | to be replaced by | | | | | |
| | | second part of CPD | | | | | |
| | | sequence | | | | | |
| CPD6B | 4 | second part of CPD | pp1ab - nsp12 | 354 | 118 | 13.954 | 14.307 |
| | | sequence | | | | | |
| WT6 | 5 | original, to be | pp1a/pp1ab - | 1482 + | 494 + | 11.969 | 14.307 |
| | | replaced by CPD | nsp7, nsp8, | 503 + | 118 = | | |
| | | sequence | nsp9, nsp10, | 354 = | 612 | | |
| | | | nsp11 and nsp12 | 2339 | | | |
| CPD6 | 6 | CPD sequence | pp1a/pp1ab - | 1482 + | 494 + | 11.969 | 14.307 |
| | | | nsp7, nsp8, | 503 + | 118 = | | |
| | | | nsp9, nsp10, | 354 = | 612 | | |
| | | | nsp11 and nsp12 | 2339 | | | |
| sWT9 | 7 | original, to be | pp1ab - | 1.146 | 382 | 20.359 | 21.504 |
| | | replaced by CPD | endoribonuclease | | | | |
| | | sequence | 2′-O- | | | | |
| | | | methyltransferase | | | | |
| sCPD9 | 8 | CPD sequence | pp1ab - | 1.146 | 382 | 20.359 | 21.504 |
| | | | endoribonuclease | | | | |
| | | | and 2′-O- | | | | |
| | | | methyltransferase | | | | |
| sWT10 | 9 | original, to be | Spike | 999 | 333 | 24.335 | 25.333 |
| | | replaced by CPD | | | | | |
| | | sequence | | | | | |
| sCPD10 | 10 | CPD sequence | Spike | 999 | 333 | 24.335 | 25.333 |

pp = polyprotein
nsp = non-structural protein

It should be noted that WT6A/CPD6A encodes only nine nucleotides (three amino acids) of nsp12 (RNA-dependent RNA polymerase, RdRp). The majority of this protein is encoded by WT6B/CPD6B.

The middle part of fragment WT6 was not recoded in fragment CPD6, because it contains a conserved regulatory RNA sequence that is essential for virus replication. Fragment WT6 encodes a −1 ribosomal frameshifting element, a so-called RNA pseudoknot structure. This structure promotes a ribosomal frameshifting, a process during which the reading frame of translation is changed at the junction between open reading frames (ORFs) 1a and 1b. During this process, a single nucleotide of the slippery sequence that is located downstream from the RNA pseudoknot structure is read twice by the translating ribosome, and the reading frame is shifted by −1 nucleotide (the ribosome slips one nucleotide backwards at the slippery sequence).

Often, translation of ORF1a terminates at the stop codon of ORF1a. However, when-1 ribosomal frameshifting occurs, translation of ORF1a continues directly to ORF1b and polyprotein (pp) 1ab is produced. Thus, the CPD6A sequence is translated into both polyproteins 1a and 1ab; but the CPD6B sequence is translated only into polyprotein 1ab.

The differences between the CPD sequences and the underlying wild type sequences are summarized in the following Table 2.

TABLE 2
Summary of difference between CPD and wild type sequences.

| | | Preserved | Altered | Different | | | |
|---|---|---|---|---|---|---|---|
| | | nucleotides | nucleotides | nucleotides | Preserved | Altered | Different |
| | SEQ | with | with | with | codons with | codons with | codons with |
| Sequence | ID | respect to | respect to | respect to | respect to | respect to | respect to |
| name | NO. | wild type | wild type | wild type | wild type | wild type | wild type |
| CPD6A | 2 | 1.144 | 338 | 22.8% | 206 | 288 | 58.3% |
| CPD6B | 4 | 270 | 84 | 23.7% | 48 | 70 | 59.3% |
| sCPD9 | 8 | 907 | 239 | 20.9% | 177 | 205 | 53.7% |
| sCPD10 | 10 | 758 | 241 | 24.1% | 132 | 201 | 60.4% |

It should be noted that the primary structure of the proteins encoded by the CPD RNA sequences is identical to the primary structure of the wild-type (original) RNA sequences. As explained above, CPD does not alter the primary structure of the resulting protein. This is summarized in Table 3.

TABLE 3
Summary of protein sequences.

| RNA | RNA | | Protein |
|---|---|---|---|
| Sequence | SEQ ID | | SEQ ID |
| name | NO. | Recoded protein | NO. |
| WT6A | 1 | ORF1a/ORF1ab - nsp7, nsp8, nsp9, | 11 |
| | | nsp10, nsp11 and nsp12 | |
| CPD6A | 2 | ORF1a/ORF1ab - nsp7, nsp8, nsp9 | |
| | | and nsp10, nsp11 and nsp12 | |
| WT6B | 3 | ORF1ab - nsp12 | 12 |
| CPD6B | 4 | ORF1ab - nsp12 | |
| sWT9 | 7 | ORF1ab - endoribonuclease and 2′- | 13 |
| | | O-methyltransferase | |
| sCPD9 | 8 | ORF1ab - endoribonuclease and 2′- | |
| | | O-methyltransferase | |
| sWT10 | 9 | Spike | 14 |
| sCPD10 | 10 | Spike | |

By recoding sequence parts CPD6A, CPD6B, SCPD9, and sCPD 10, different fragments of the SARS-CoV-2 genome were generated that form part of a live attenuated SARS-CoV-2 in an embodiment. These fragments are listed in the following Table 4.

TABLE 4
Genome fragments of SARS-CoV-2 comprising recoded sequence parts.

| | | SEQ | | |
|---|---|---|---|---|
| Genome fragment | Length | ID | Accession | Website providing access to |
| name | (nucleotides) | NO. | number | sequence |
| Fragment 6 | 2.943 | 15 | MZ064535 | http://getentry.ddbj.nig.ac.jp/getentry/ |
| comprising CPD6A | | | | na/MZ064535?filetype=html |
| and CPD6B | | | | |
| Fragment 9 | 3.178 | 16 | MZ064545 | http://getentry.ddbj.nig.ac.jp/getentry/ |
| comprising sCPD9 | | | | na/MZ064545?filetype=html |
| Fragment 10 | 2.966 | 17 | MZ064546 | http://getentry.ddbj.nig.ac.jp/getentry/ |
| comprising sCPD10 | | | | na/MZ064546?filetype=html |

In an aspect, the solution relates to a live attenuated severe acute respiratory syndrome coronavirus 2 (SARS-CoV-2) comprising the polynucleotide according the above explanations.

In an embodiment, the live attenuated SARS-CoV-2 has a nucleic acid sequence being at least 98%, in particular at least 99%, in particular 100% identical to SEQ ID NO. 18.

In an embodiment, the SARS-CoV-2 has a nucleic acid sequence being at least 98%, in particular at least 99%, in particular 100% identical to SEQ ID NO. 19.

In an aspect, the solution relates to a pharmaceutical composition comprising the live attenuated SARS-CoV-2 according to any of the preceding explanations. Such a pharmaceutical composition can further comprise auxiliary substances like adjuvants, e.g., for enhancing an immune response of a patient. Appropriate adjuvants are potassium alum; aluminium hydroxide; aluminium phosphate; calcium phosphate hydroxide; aluminum hydroxyphosphate sulfate; paraffin oil; propolis; killed bacteria of the species Bordetella pertussis or Mycobacterium bovis; plant saponins from Quillaja, soybean, and/or Polygala senega; cytokines IL-1, IL-2, and/or IL-12; as well as Freund's complete adjuvant.

In an aspect, the solution relates to the further medical use of such a pharmaceutical composition as a vaccine.

In an aspect, the solution relates to a method for preparing a vaccine from such a pharmaceutical composition.

In an aspect, the solution relates to a method of vaccinating a human or animal patient in need thereof. The animal patient is in particular a non-human mammal such as a rodents, canines, felines, or mustelids. This method comprises the step of administering a pharmaceutical composition according to the preceding explanations to the patient.

In an embodiment, the administration is performed by an intranasal administration, an oral administration, a parenteral administration such as a subcutaneous injection, an intramuscular injection, an intravenous injection, an intraperitoneal injection, an intravenous infusion, or an intraperitoneal infusion. An intranasal or oral administration is particularly appropriate. By these routes of administration, the live attenuated SARS-CoV-2 is presented to the body of the patient to be vaccinated in the same or a similar way as in case of a natural exposure to the virus.

In an embodiment, the vaccination is performed by administering the pharmaceutical composition in a dose comprising between 1*10³ and 1*10⁸ focus-forming units (FFU), in particular between 1*10⁴ and 1*10⁷ FFU, in particular between 1*10⁵ and 1*10⁶ FFU, of the live attenuated SARS-CoV-2. The dose is chosen such that the pharmaceutical composition is well tolerated by the patient but evokes an immune response that gives protection to the patient against an infection or a severe course of an infection with SARS-CoV-2. In embodiment, the dose is one of the lowest protective dose and the highest tolerable dose or lies between the lowest protective dose and the highest tolerable dose.

Various factors can influence the dose used for a particular application. For example, the frequency of administration, duration of treatment, preventive or therapeutic purpose, the use of multiple treatment agents, route of administration, previous therapy, the patient's clinical history, the discretion of the attending physician and severity of the disease, disorder and/or condition may influence the required dose to be administered.

As with the dose, various factors can influence the actual frequency of administration used for a particular application. For example, the dose, duration of treatment, use of multiple treatment agents, route of administration, and severity of the disease, disorder and/or condition may require an increase or decrease in administration frequency.

In some cases, an effective duration for administering the pharmaceutical composition of the solution (and any additional therapeutic agent) can be any duration that reduces the severity, or occurrence, of symptoms of the disease, disorder and/or condition to be treated without producing significant toxicity to the subject. Multiple factors can influence the actual effective duration used for a particular treatment. For example, an effective duration can vary with the frequency of administration, effective amount, use of multiple treatment agents, route of administration, and severity of the disease, disorder and/or condition being treated.

In an embodiment, the pharmaceutical preparation is administered to the patient at least two times, wherein the second administration is separated from the first administration by a first time period. In this context, the first time period lies in a range of from 2 weeks to 36 months, in particular of from 3 weeks to 30 months, in particular of from 4 weeks to 24 months, in particular of from 5 weeks to 21 months, in particular of from 6 weeks to 18 months, in particular of from 7 weeks to 15 months, in particular of from 8 weeks to 12 months, in particular of from 9 weeks to 10 months, in particular of from 10 weeks to 8 months, in particular of from 12 weeks to 6 months, in particular of from 13 weeks to 4 months.

In an embodiment, the pharmaceutical preparation is administered to the patient temporally offset to administering a different vaccine (such as, e.g., a vector-based vaccine, an mRNA-based vaccine, a protein-based vaccine) to the patient, i.e., after or before vaccinating the patient with the different vaccine. In this context, the administration of the pharmaceutical composition is offset to the administration of the different vaccine by a second time period. In this context, the second time period lies in a range of from 2 weeks to 36 months, in particular of from 3 weeks to 30 months, in particular of from 4 weeks to 24 months, in particular of from 5 weeks to 21 months, in particular of from 6 weeks to 18 months, in particular of from 7 weeks to 15 months, in particular of from 8 weeks to 12 months, in particular of from 9 weeks to 10 months, in particular of from 10 weeks to 8 months, in particular of from 12 weeks to 6 months, in particular of from 13 weeks to 4 months.

In an aspect, the solution relates to a vector comprising the polynucleotide according to the above explanations.

The term “vector”, as used herein, refers to a nucleic acid molecule, capable of transferring or transporting itself and/or another nucleic acid molecule into a cell. The transferred nucleic acid is generally linked to, i.e., inserted into, the vector nucleic acid molecule. A vector may include sequences that direct autonomous replication in a cell, or may include sequences sufficient to allow integration into host cell DNA. In some embodiments, the vector described herein is a vector selected from the group of plasmids (e.g., DNA plasmids or RNA plasmids), shuttle vectors, transposons, cosmids, artificial chromosomes (e.g. bacterial, yeast, human), and viral vectors.

In some embodiments, the vector described herein is used in combination with at least one transfection enhancer, e.g., a transfection enhancer selected from the group of oligonucleotides, lipoplexes, polymersomes, polyplexes, dendrimers, inorganic nanoparticles and cell-penetrating peptides.

In an aspect, the solution relates to a host cell comprising the polynucleotide according to the above explanations.

The term “host cell”, as used herein, refers to a cell into which exogenous nucleic acid has been introduced, including the progeny of such a cell. Host cells include “transformants” and “transformed cells”, which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

In an embodiment, the host cell described herein comprises at least one cell type selected from the group of Chinese hamster ovary (CHO), Vero, Vero E6, Vero TMPRSS, MRC 5, Per.C6, PMK, and WI-38.

In an aspect, the solution relates to a method for the production of a virus. This method comprises the steps of a) culturing a host cell according to the preceding paragraph; and b) isolating a virus, wherein the virus is a live attenuated SARS-CoV-2.

All embodiments of the polynucleotide can be combined in any desired way and can be transferred either individually or in any arbitrary combination to the live attenuated SARS-CoV-2, to the pharmaceutical composition, its use, to the method of vaccinating the patient, to the vector, to the host cell, and to the method of producing a virus. All embodiments of the live attenuated SARS-CoV-2 can be combined in any desired way and can be transferred either individually or in any arbitrary combination to the polynucleotide, to the pharmaceutical composition, its use, to the method of vaccinating the patient, to the vector, to the host cell, and to the method of producing a virus. Likewise, all embodiments of the pharmaceutical composition can be combined in any desired way and can be transferred either individually or in any arbitrary combination to the polynucleotide, to the live attenuated SARS-CoV-2, the use of the pharmaceutical composition, to the method of vaccinating a patient, to the vector, to the host cell, and to the method of producing a virus. All embodiments of the use of the pharmaceutical preparation can be combined in any desired way and can be transferred either individually or in any arbitrary combination to the polynucleotide, to the live attenuated SARS-CoV-2, to the pharmaceutical preparation, to the method of vaccinating a patient, to the vector, to the host cell, and to the method of producing a virus. Finally, all embodiments of the method of vaccinating a patient can be combined in any desired way and can be transferred either individually or in any arbitrary combination to the polynucleotide, to the live attenuated SARS-CoV-2, to the pharmaceutical preparation, to the use of the pharmaceutical preparation, to the vector, to the host cell, and to the method of producing a virus.

### BRIEF DESCRIPTION OF THE DRAWINGS

Further details of aspects of the solution will be explained in the following making reference to exemplary embodiments and accompanying Figures.

FIGS. 1A to 1D schematically depict the native structure and exemplary recoding of the SARS-CoV-2 genome.

FIGS. 2A and 2B illustrate the areas of infected cell foci and multi-step growth kinetics of the parental and recoded viruses in Vero E6 cells.

FIGS. 3A to 3P illustrate the attenuation of recoded SARS-CoV-2 mutants in Syrian hamsters.

FIGS. 4A to 4N illustrate the lung histopathology of infected Syrian hamsters three days after vaccination.

FIGS. 5A to 5V illustrate the lung histopathology of vaccinated Syrian hamsters on day 2 to 14 after challenge.

FIG. 6 shows titers of SARS-CoV-2 neutralizing antibodies in vaccinated Syrian hamsters;

FIGS. 7A to 7O illustrate that recoded SARS-CoV-2 mutant sCPD9s is strongly attenuated in Roborovski dwarf hamsters.

FIG. 8 shows representative images of virus foci formed by the parental SARS-CoV-2 (WT) and recoded viruses.

FIGS. 9A and 9B show a Western Blot analysis indicating that sCPD10 virus produces less spike protein in infected Vero E6 cells than parental or sCPD9 viruses.

FIGS. 10B to 10G illustrate that live attenuated virus vaccine candidate sCPD9s is strongly attenuated and protects Roborovski dwarf hamsters from challenge with B.1, B.1.1.7, and B.1.351 viruses (FIG. 10A is intentionally omitted).

FIGS. 11A to 11T illustrate the pulmonary histopathology of mock/sCPD9-vaccinated and challenged Roborovski dwarf hamsters at different time points.

FIGS. 12A to 12C illustrate the sensitivity of SARS-CoV-2 variants B.1, B.1.1.7 (Alpha), B.1.351 (Beta), B.1.128.1 (Gamma), and B.1.617.2 (Delta) to neutralization by antibodies in sera of sCPD9-vaccinated Roborovski dwarf hamsters.

### DETAILED DESCRIPTION

### First Exemplary Embodiment

The inventors generated a series of recoded SARS-CoV-2 mutants, characterized them in cultured cells and also in vivo using the Syrian and Roborovski hamster models. It was proven that a single-dose intranasal immunization with attenuated live viruses can elicit strong immune responses and offer complete protection against SARS-CoV-2 challenge in a robust small animal model of COVID-19.

### Vaccine Design

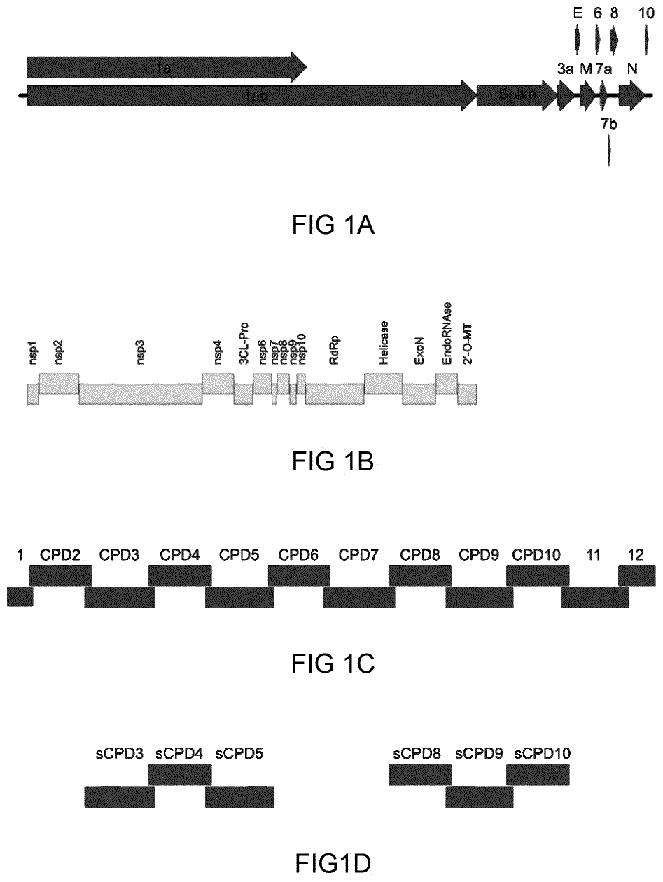

The inventors' goal was to generate attenuated SARS-CoV-2 vaccine candidates through large-scale recoding of the SARS-CoV-2 genome by CPD (Eschke et al., 2018; Groenke et al., 2020; Khedkar et al., 2018; Kunec and Osterrieder, 2016). To achieve virus attenuation in humans, the inventors recoded the genome of SARS-CoV-2 with codon pairs that are the most underrepresented in human genes (Groenke et al., 2020). The genetically modified SARS-CoV-2 mutants were produced using a recently established reverse genetics system of SARS-CoV-2 (Thi Nhu Thao et al., 2020) (FIGS. 1A to 1D). The system relies on 12 subgenomic fragments of the SARS-CoV-2 genome, which are assembled into a single yeast/bacterial artificial chromosome (YAC/BAC) by transformation-associated recombination (TAR) cloning in Saccharomyces cerevisiae (Noskov et al., 2002). The subgenomic fragments are approximately 3,000 bp long, and the neighboring fragments overlap each other by approximately 300 bp to enable the assembly of the SARS-CoV-2 infectious clone by homologous recombination.

In this context, FIGS. 1A to 1D illustrate the structure and recoding of the SARS-CoV-2 genome.

FIG. 1A illustrates that the SARS-CoV-2 genome is a single-stranded, positive-sense RNA molecule of about 30,000 nucleotides (nt), which encodes 11 canonical ORFs. “3CL-Pro” denotes 3C-like proteinase; “RdRp” denotes RNA-dependent RNA polymerase; “ExoN” denotes 3′-to-5′ exoribonuclease; “EndoRNAse” denotes endoribonuclease; and “2′-O-MT” denotes 2′O-ribose methyltransferase.

As illustrated in FIG. 1B, after infection, ORF1a/1ab is directly translated and cleaved into 15 proteins of the replication-transcription complex.

As illustrated in FIG. 1C, recoded SARS-CoV-2 mutants were constructed using a recently established reverse genetics system of SARS-CoV-2, which consists of 12 subgenomic fragments. Fragments 1, 11 and 12 were not recoded. The dark grey boxes indicate recoded sequences CPD2-10, and the light grey boxes indicate parental, non-recoded sequences in the respective fragments 2-10. The frame-shifting element contained in fragment 6 and the transcription regulatory sequence (TRS) of the spike gene in fragment 9 were excluded from the recoding process (light grey boxes present between two dark grey boxes in CPD6 and CPD9).

As shown in FIG. 1D, the dark grey boxes indicate recoded sequences sCPD3-5 and sCPD8-10 in different subgenomic fragments.

To preserve the full compatibility with the available reverse genetics system, the inventors recoded only SARS-CoV-2 sequences that were not present in the overlapping parts of the subgenomic fragments (approximately 2,500 bp in each recoded fragment) (FIGS. 1A to 1D). This design enabled the inventors to generate a wide variety of SARS-CoV-2 mutants, carrying a single or more recoded fragments.

The inventors recoded nine fragments of the SARS-CoV-2 reverse genetics system (fragments 2-10). Fragments 1 and 12, which are relatively short, 591 and 1,812 bp respectively, and fragment 11, which contains many short ORFs, were excluded from the recoding. To ensure that mutant viruses are replication-competent, two genomic regions containing essential cis-acting RNA elements were excluded from the recoding: the frame-shifting element carried by fragment 6 and the transcription regulatory sequence (TRS) of the spike gene within fragment 9. In addition, the first 500 bp of ORF1a located in fragment 2 were not recoded either.

### Recovery of Recoded Virus Mutants

The inventors attempted to rescue infectious progeny from the recoded SARS-CoV-2 constructs by two different methods—directly from DNA in Vero E6 cells and from viral RNA in BHK 21 cells. To enable recovery from DNA, the genome of the SARS-CoV-2 was placed under the control of the immediate-early promoter of the human cytomegalovirus. The inventors rescued infectious viruses that carried recoded fragments 2 and 6 (thereafter labeled as CPD2 and CPD6). It is known that extensive recoding by CPD can lead to a lethal phenotype of recoded mutants (Eschke et al., 2018). The inventors suspected that the extent of recoding in constructs that did not produce infectious viruses was too high and, therefore, constructed additional mutants with shorter deoptimized sequences (FIGS. 1A to 1D). In total, the inventors engineered six SARS-CoV-2 mutant constructs that carried shorter, approximately 1,000 bp-long recoded sequences (SCPD3, SCPD4, sCPD5, sCPD8, sCPD9, and sCPD10). As predicted, reducing the extent of deoptimization led to the rescue of several additional recoded SARS-CoV-2 viruses. Specifically, of the six constructs, the inventors rescued four mutant viruses by means of DNA transfection: sCPD3, sCPD4, sCPD9, and sCPD10.

### Results

### Characterization of Recoded SARS-CoV-2 Mutants in Cell Culture

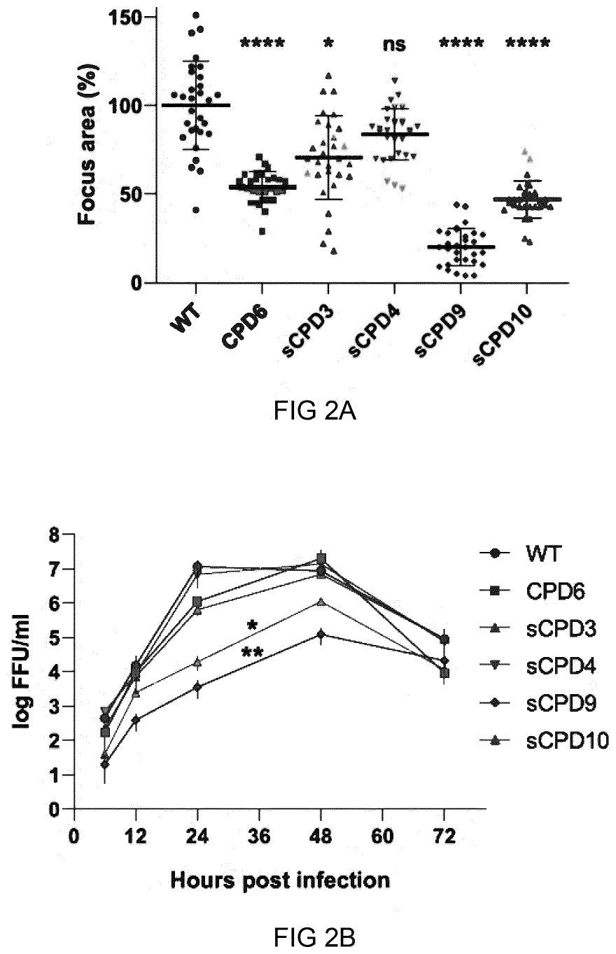

The morphological changes induced by infection on Vero E6 cells were highly variable among mutant viruses. While CPD6, sCPD3, and sCPD4 viruses produced readily visible plaques on Vero E6 cells within 24 hours of infection (hpi), sCPD9 and sCPD10 viruses did not produce visible plaques or discernable cytopathic effect (CPE) on Vero E6 cells in the first 72 hpi. Therefore, the inventors determined virus titers and spread of virus mutants in cell culture monolayers by what the inventors refer to as assays to assess virus induced foci (focus-forming assays) and is reliant on the identification of infected cells by immunofluorescence tests under semisolid medium (Avicel). The recoded viruses CPD6, sCPD3, sCPD9 and sCPD10 formed significantly smaller foci of infected cells than the parental virus (FIG. 2A, FIG. 8). The foci produced by the sCPD3 and sCPD4 viruses were slightly smaller than those formed by the parental virus. On average, they were about 65% and 80% of the size of those formed by the parental virus. In contrast, foci formed by the CPD6, sCPD9 and sCPD10 viruses were significantly smaller, accounting on average for about 50%, 20% and 45% of the size of the parental virus, respectively.

In this context, FIGS. 2A and 2B show the areas of infected cell foci and multi-step growth kinetics of the parental and recoded viruses in Vero E6 cells.

FIG. 2A shows the relative areas of infected cell foci at 48 hpi. Focus areas were normalized against the average focus size caused by the parental virus. Bars show geometric mean and SD. P-values were calculated using Kruskal-Wallis test with Dunn's post-hoc test, *P=0.0135 and ****P<0.0001. See also FIG. 8.

FIG. 2B shows multi-step growth kinetics of the parental and recoded viruses. Vero E6 cells were infected with the parental or recoded viruses at a multiplicity of infection (MOI) of 0.01. Cell culture medium was collected 6, 12, 24, 48 and 72 hpi, and virus titers were determined by focus-forming assay. Data are represented as means of three independent biological replicates±SD. Comparison of growth curves by Friedman test with Dunn's post-hoc test showed that sCPD9 and sCPD10 viruses replicated significantly worse than the parental virus, *P=0.0264 and **P=0.0049, respectively.

FIG. 8 shows representative images of virus foci formed by the parental SARS-CoV-2 (WT) and recoded viruses. The bar represents 1 mm.

These multi-step growth kinetics in Vero E6 cells showed that recoded viruses replicated with variable efficiency in Vero E6 cells (FIG. 2B). The replication kinetics of the CPD6, sCPD3 and sCPD4 viruses were comparable to those of the parental virus. In contrast, the sCPD9 and sCPD10 viruses replicated at significantly reduced levels at all assessed time points. The mean titers of sCPD9 and sCPD10 viruses were 100-1,000 times lower than those of the parental virus at 24 hpi and 10-100 times lower at 48 hpi.

To assess the genetic stability of the recoded viruses, the inventors passaged the two most attenuated viruses sCPD9 and sCPD10 serially 10 times in Vero E6 cells at an MOI of 0.1. After 10 passages, the viruses did not appear to have undergone notable phenotypic changes, as they still produced only small foci on Vero E6 cells and did not produce readily observable CPE within 72 hpi. The inventors also analyzed the genomes of the passaged viruses by RT-PCR and Sanger sequencing, and the inventors did not detect any mutations in the recoded regions after serial passage.

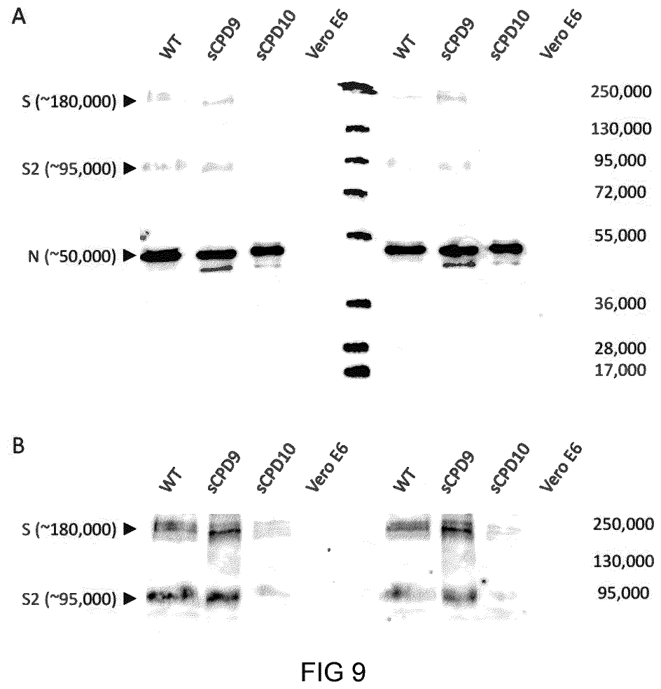

While recoding by codon pair deoptimization does not affect the amino acid sequence of the encoded proteins, it extensively alters the underlying genomic sequence. Suboptimal codon pairs reduce the mRNA stability and translation efficiency of recoded, codon pair deoptimized genes (Groenke et al., 2020; Mueller et al., 2010). This in turn reduces protein production and leads to virus attenuation. To determine whether reduced protein production from recoded genes could be the reason for attenuation of mutant viruses, the inventors examined the production of spike and nucleocapsid proteins in Vero E6 cells infected with wild-type (WT), sCPD9, or sCPD10 viruses. Because the sCPD10 virus carries a codon pair-deoptimized spike gene (FIGS. 1A to 1D), it is expected to produce comparatively less spike protein than either the WT or the sCPD9 viruses. Indeed, spike protein production was significantly reduced in cells infected with sCPD10 virus compared with cells infected with WT or sCPD9 viruses (FIGS. 9A and 9B), indicating that CPD reduced protein production of the target gene.

In this context, FIG. 9 illustrates that sCPD10 virus produces less spike protein in infected Vero E6 cells than parental or sCPD9 viruses. FIG. 9A shows a Western blot analysis of viral protein production in infected cells by SARS-CoV-2 (WT), sCPD9 and sCPD10 viruses. Vero E6 cells were infected with WT, sCPD9 or sCPD10 viruses. After 48 h, infected cells were lysed, cell lysates separated by SDS-PAGE under reducing conditions, and proteins were transferred to PVDF membranes. The membranes were cut into two parts. The upper membrane part, containing proteins with higher molecular weight, was incubated with a mouse monoclonal anti-S antibody. Note that the antibody binds to the S2 subunit of the spike protein and thus can recognize both the uncleaved spike protein (MW˜180,000) and its S2 subunit (MW˜95,000). The lower membrane part containing lower molecular weight proteins was incubated with a mouse monoclonal antibody recognizing the N protein (MW˜90,000).

FIG. 9B shows an image of the upper part of the membrane containing the uncleaved spike protein and its subunits, obtained after a longer exposure. Data are representative of three independent experiments.

### Vaccination of Syrian Hamsters and Challenge Infection Setup

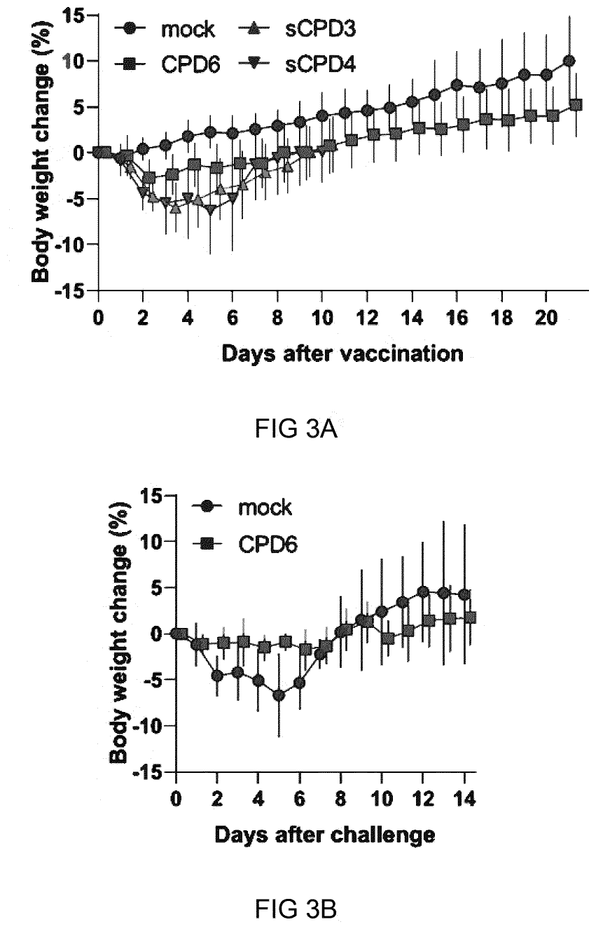

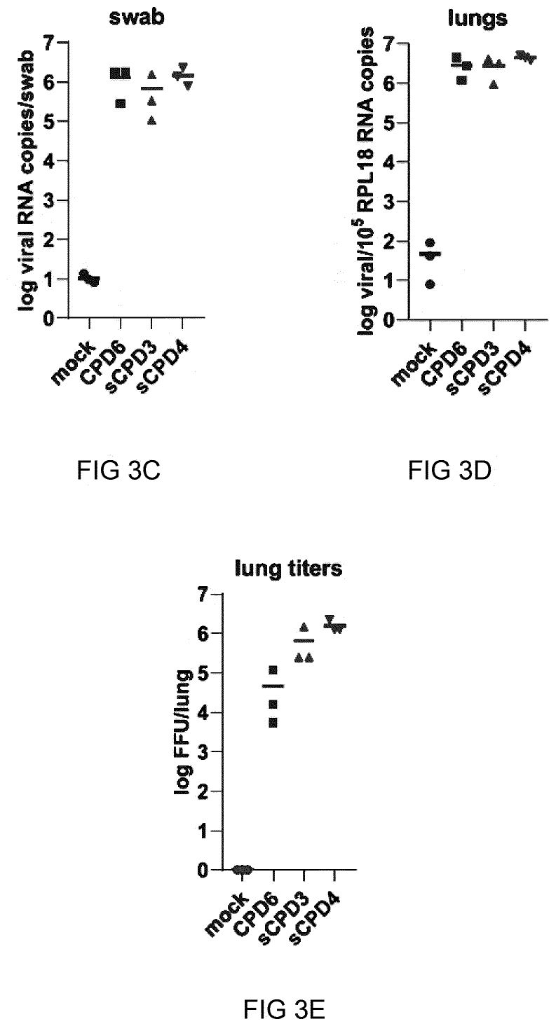

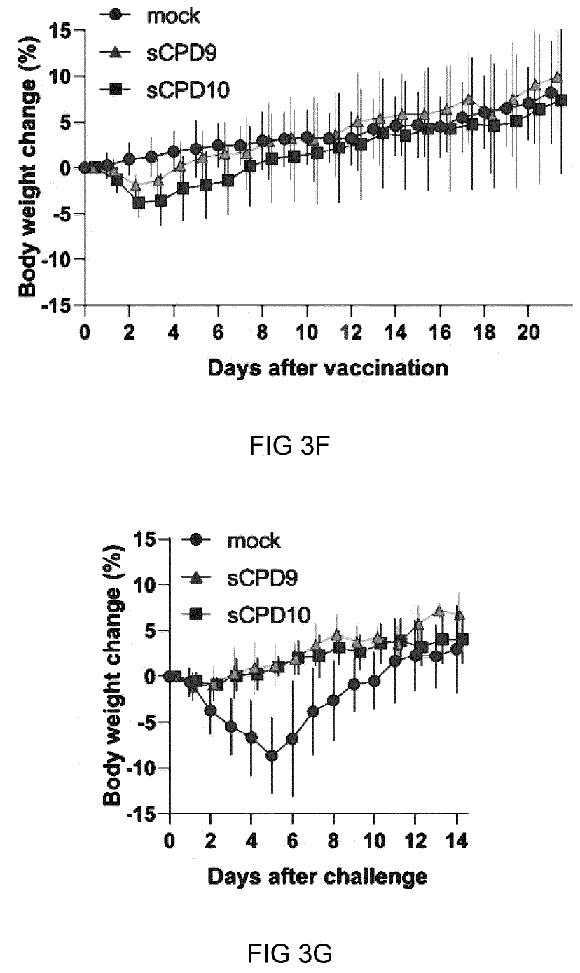

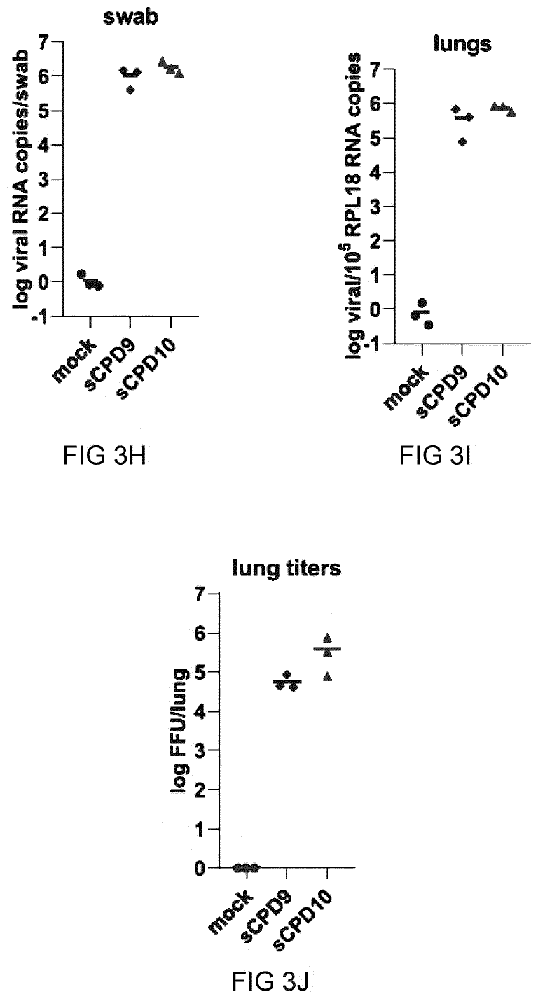

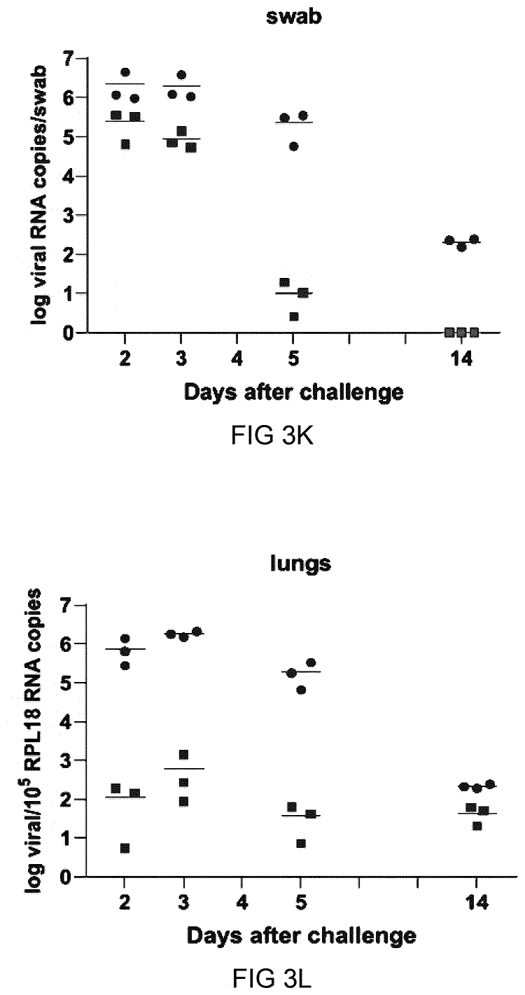

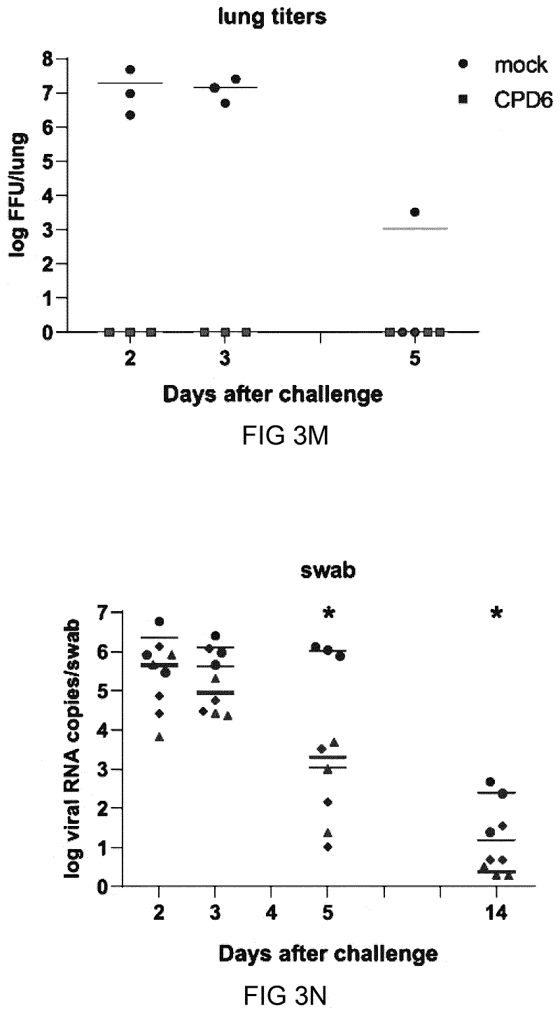

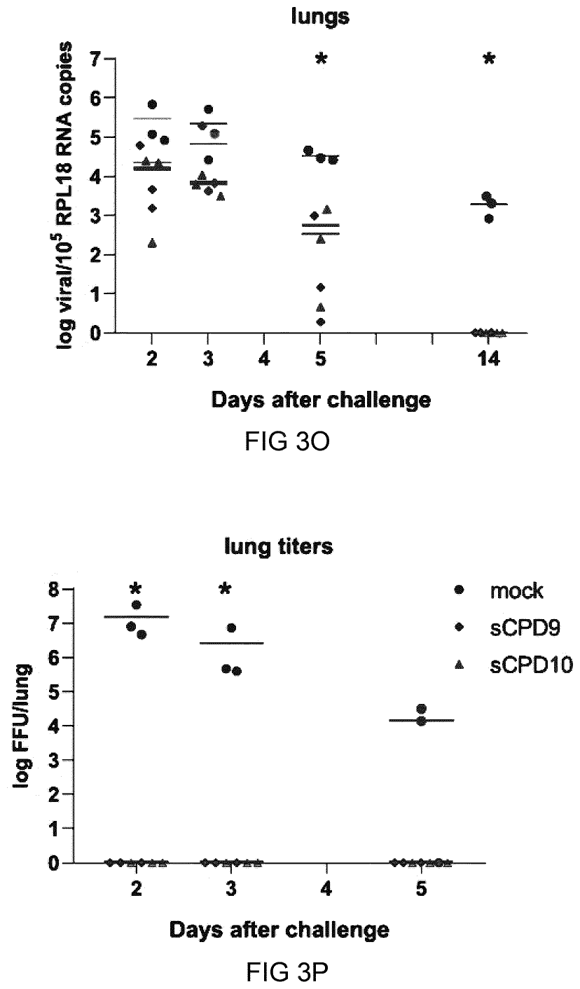

The degree of attenuation of the recoded SARS-CoV-2 mutants was assessed in the Syrian hamster model by a single-dose intranasal infection (FIGS. 3A to 3P). Hamsters were kept in individually ventilated cages and randomly assigned to four groups of 15 animals. Animals were either mock-vaccinated or vaccinated with 1×10⁴ or 1×10⁵ FFU of recoded virus on day 0. Twenty-one days after vaccination, animals were challenged by intranasal infection with 1×10⁵ FFU of the WT SARS-CoV-2, variant B.1 (Wolfel et al., 2020). For the duration of the vaccination experiment (35 days), body weights and clinical signs were recorded daily. Three animals of each group were euthanized on day 3 after vaccination, and on day 23, 24, 26 and 35 after vaccination (corresponding to day 2, 3, 5 and 14 after challenge) to determine the degree of virus replication in different organs and to assess pathological changes in the lungs.

FIGS. 3A to 3P show the attenuation of recoded SARS-CoV-2 mutants in Syrian hamsters. The attenuation level of recoded virus vaccine candidates was evaluated in two sequential trials. In the first trial, Syrian hamsters were either mock-vaccinated or vaccinated with CPD6, sCPD3, or sCPD4 viruses (FIGS. 3A to 3E and 3K to 3M). In the second trial, Syrian hamsters were either mock-vaccinated, or vaccinated with sCPD9 or sCPD10 viruses (FIGS. 3F to 3J and 3N to 3P). Twenty-one days after vaccination, all animals were challenged by infection with WT SARS-CoV-2.

FIGS. 3A, 3B, 3F, and 3G show the change in body weight of Syrian hamsters after vaccination (n=15; FIGS. 3A and 3F) and challenge (n=12; FIGS. 3B and 3G). Data are represented as means±SD.

FIGS. 3C, 3D, 3H, and 3I show the viral load in the upper (FIGS. 3C and 3H) and lower (FIGS. 3D and 3H) respiratory tract on day 3 after vaccination.

FIGS. 3E and 3J illustrate the number of infectious virus particles detected in 50 mg of lung tissue on day 3 after vaccination.

FIGS. 3K, 3L, 3N, and 3O illustrate the viral load in the upper (FIGS. 3K and 3N) and lower (FIGS. 3L and 3O) respiratory tract of animals on days 2, 3, 5 and 14 after challenge.

FIGS. 3M and 3P show the amount of infectious virus detected in 50 mg of lung tissue on days 2, 3 and 5 after challenge. The Kruskal-Wallis test was used to determine whether the differences in viral load among the different groups were significant (FIGS. 3N to 3P), *P<0.05.

Due to space limitations, the inventors examined the attenuation of five recoded viruses in two animal trials. In the first trial, hamsters were vaccinated with 1×10⁵ FFU of the recoded viruses CPD6, sCPD3, or sCPD4. In the second trial, hamsters were vaccinated with 1×10⁴ FFU of the recoded viruses sCPD9, or sCPD10, a 10-fold lower dose than in the first vaccination trial. The inventors used a lower dose because initially the inventors were initially unable to grow the sCPD9 and sCPD10 mutants to sufficiently high levels, as the peak titers of these mutants in Vero E6 cells were 10-100 times lower than those of the parental virus (FIG. 2B).

Recoded Viruses CPD6, sCPD9 and sCPD10 are Attenuated in Syrian Hamsters

On day 3 after vaccination, all tested viruses replicated efficiently in the upper and lower respiratory tract of vaccinated hamsters, as determined by the viral load in oropharyngeal swabs (FIGS. 3C and 3H) and lung tissues (FIGS. 3D and 3I). Unexpectedly, despite weaker replication in Vero E6 cells, the recoded viruses sCPD9 and sCPD10 replicated efficiently in the upper respiratory tract on day 3 after vaccination (FIG. 3H). In fact, the quantity of detected RNA copies in oropharyngeal swabs (˜10⁶ RNA copies/swab) was comparable among all recoded viruses (FIGS. 3C and 3H), and is also comparable to the viral loads of the WT virus in mock-infected animals on day 3 after challenge (FIGS. 3K and 3N). These results indicate that all recoded viruses replicated with comparable efficiency in the upper respiratory tract of infected animals, although the viruses showed markedly different replication capacity in cultured Vero E6 cells and although the animals were vaccinated with dissimilar doses of recoded viruses.

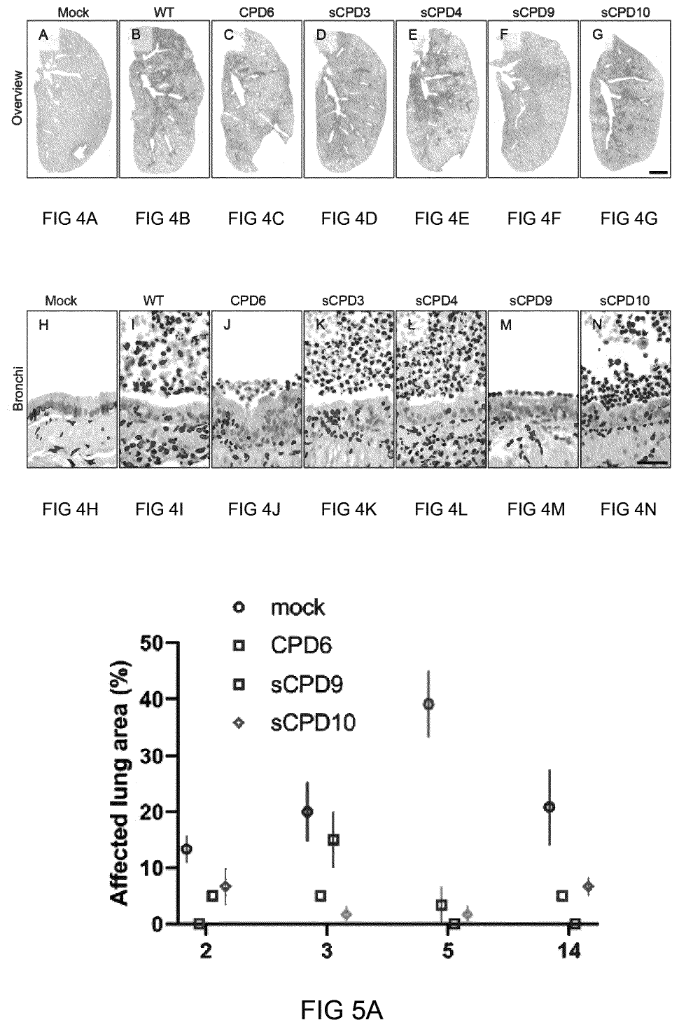

Successful reisolation of replicating virus from the lungs of vaccinated hamsters on day 3 after vaccination confirmed that all recoded viruses also replicated in the lungs of vaccinated animals (FIGS. 3E and 3J). Virus titers were lower in the lungs of hamsters vaccinated with CPD6 virus than in the lungs of hamsters vaccinated with either sCPD3 or sCPD4 viruses (FIG. 3E), indicating that CPD6 was the most attenuated virus of the three viruses examined. The inventors detected less infectious virus in the lungs of hamsters vaccinated with sCPD9 virus than in those vaccinated with sCPD10 virus (FIG. 3J), indicating that sCPD9 was more attenuated than sCPD10 virus in vivo. In general, lung virus titers correlated well with virus foci sizes (FIG. 2A) and also with lung histopathology on day 3 after vaccination (FIGS. 4A to 4N).

In this context, FIGS. 4A to 4N illustrate the lung histopathology of infected Syrian hamsters three days after vaccination.

FIGS. 4A to 4N show representative whole cross-sectional scans of left lung lobes (upper row, FIGS. 4A to 4G) and micrographs of bronchial epithelium (bottom row, FIGS. 4H to 4N) of formalin-fixed, paraffin embedded, hematoxylin and eosin-stained tissues. Hamsters were either mock-vaccinated (Mock), infected with SARS-CoV-2 (WT) or vaccinated with viruses CPD6, SCPD3, sCPD4, sCPD9 or sCPD10. Bars: 1 mm (FIG. 4A to 4G) or 100 μm (FIGS. 4H to 4N).

Hamsters vaccinated with sCPD3 and sCPD4 viruses lost significant body weight from day 2 after inoculation (FIG. 3A), and histological examination of the lungs on day 3 revealed a pronounced necrosuppurative bronchointerstitial pneumonia (FIGS. 4A to 4N). Four to five days after vaccination, hamsters reached the peak of clinical symptoms and slowly began to recover, resulting in weight gain. As the clinical and histological signs were reminiscent of those caused by infection with pathogenic WT SARS-CoV-2 (Osterrieder et al., 2020), the inventors discontinued the vaccination trial for sCPD3 and sCPD4 viruses on day 10 after vaccination.

The CPD6 virus was moderately attenuated in vivo. Individual hamsters lost up to 6% of their body weight within the first 3 days after vaccination. Vaccinated animals weighed on average 0.4% less on day 1 and 2.8% less on day 2 after vaccination (FIG. 3A). From day 3, the animals started to regain the lost weight. In contrast, average daily weight loss was significantly higher in mock-vaccinated hamsters after challenge infection with WT virus in both vaccination trials (FIGS. 3B and 3G). The average body weight of hamsters decreased by 1.1, 5.0, 7.1, 7.6 and 9.6% during the first five days after challenge infection compared to the day of challenge. In addition, individual hamsters lost up to 14% of their weight during this period (FIGS. 3B and 3G). In both trials, hamsters began to gain weight from day 5 after challenge.

Because Syrian hamsters were inoculated with a 10-fold lower virus dose in the second animal trial relative to the first trial, the degree of attenuation of sCPD9 and sCPD10 viruses cannot be directly compared with that of CPD6, sCPD3, or sCPD4 viruses or with that of WT viruses after challenge of mock-vaccinated hamsters in either experiment. Nevertheless, the obtained results show how pathogenic sCPD9 and sCPD10 viruses are to Syrian hamsters when vaccinated with 1×10⁴ FFU/animal, and whether this dose can protect vaccinated Syrian hamsters from challenge infection with WT virus.

Syrian hamsters vaccinated with sCPD9 and sCPD10 viruses did not develop any adverse reactions or signs consistent with SARS-CoV-2 infection. Hamsters lost weight during the first 2 days after vaccination, but the average daily weight loss was minor. The weight of animals vaccinated with sCPD9 virus decreased by average of 0.3% on day 1 and 1.9% on day 2 after vaccination (FIG. 3F). In hamsters vaccinated with sCPD10 virus, weight loss was more pronounced but still moderate. Animals weighed an average of 1.3% less on day 1 and 3.8% less on day 2 after vaccination compared to baseline weight. The inventors recorded weight gain in both groups on day 3 following vaccination, and when animals had fully recovered, the average daily gains were comparable to those of the mock-vaccinated group.

### Vaccination of Syrian Hamsters Provides Complete Protection Against SARS-CoV-2 Challenge

Vaccination with the CPD6 virus induced strong protective immunity against the challenge with the WT SARS-CoV-2. The CPD6-immunized animals did not lose weight (FIG. 3B) and showed no signs of disease after the challenge. CPD6-vaccinated animals had lower viral loads in the upper and lower airways on days 2, 3, 5 and 14 after challenge than mock-vaccinated animals (FIGS. 3K and 3L). To better understand efficacy of vaccination, the inventors also performed virus isolation from lung tissue (FIG. 3M). The inventors were able to detect high titers of replicating viruses from lung tissue of mock-vaccinated but not CPD6-vaccinated animals, suggesting that the vaccination with live attenuated CPD6 virus induced an immunity that efficiently prevented replication of the challenge virus in the lungs.

Similarly, vaccination with the sCPD9 and sCPD10 viruses induced strong protective immunity against challenge infection with WT SARS-CoV-2. The vaccinated animals did not lose weight and showed no clinical signs of disease after infection. RT-qPCR analysis indicated active virus replication in the upper airways of infected animals (FIG. 3N). On day 5 after challenge, virus loads were approximately 1,000-fold lower when compared to the mock-vaccinated group. Similarly, virus loads in the lower respiratory tract were significantly lower in the vaccinated animals compared to the mock-vaccinated animals (FIG. 3O). To evaluate vaccine efficacy, the inventors also tried to isolate the virus from lung tissue of challenged animals. As expected, while the inventors could readily isolate the virus from the tissue samples of the mock-vaccinated animals, no infectious virus was isolated from the lung samples of sCPD9- and sCPD10-vaccinated animals (FIG. 3P). However, these animals showed persistent viral loads in the throat, indicating active virus replication (FIG. 3N).

### Vaccination Prevents Major Tissue Damage

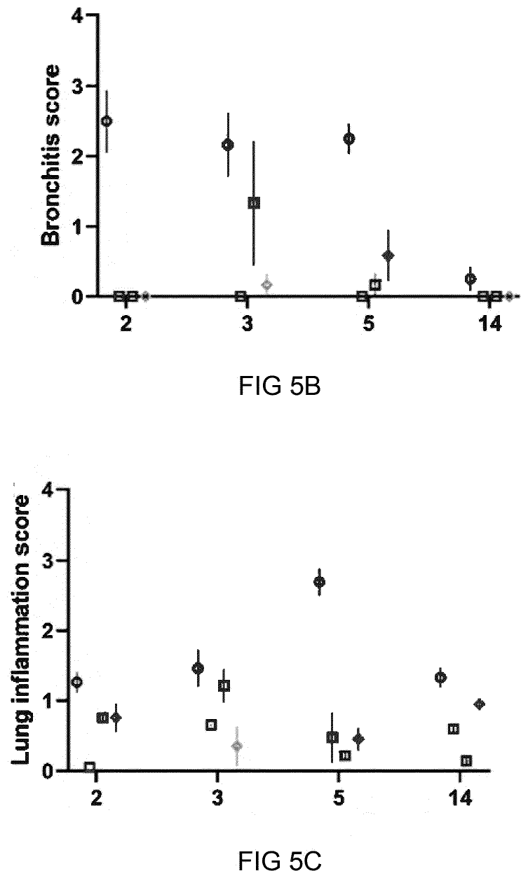

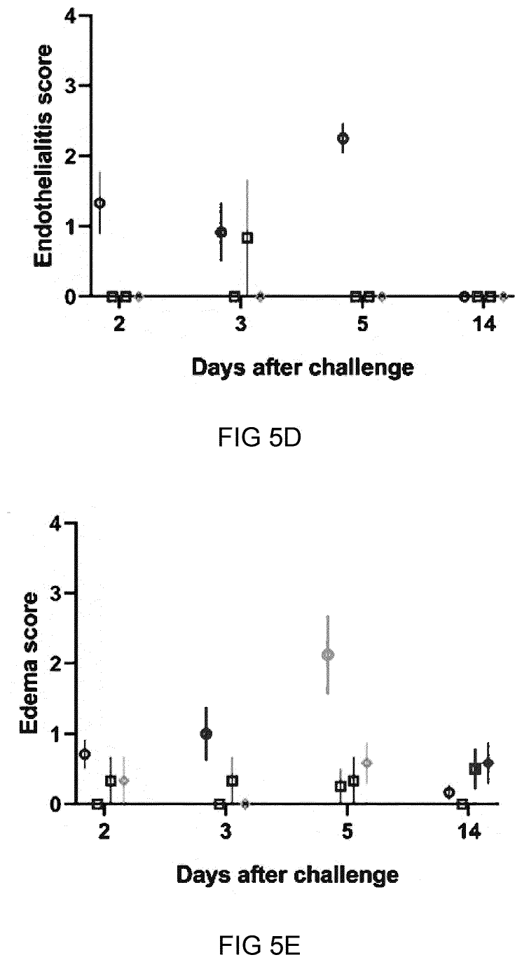

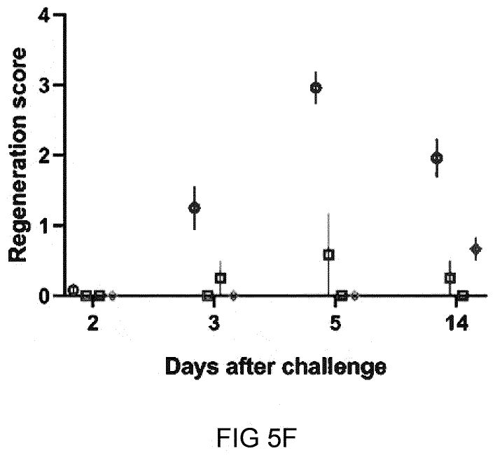

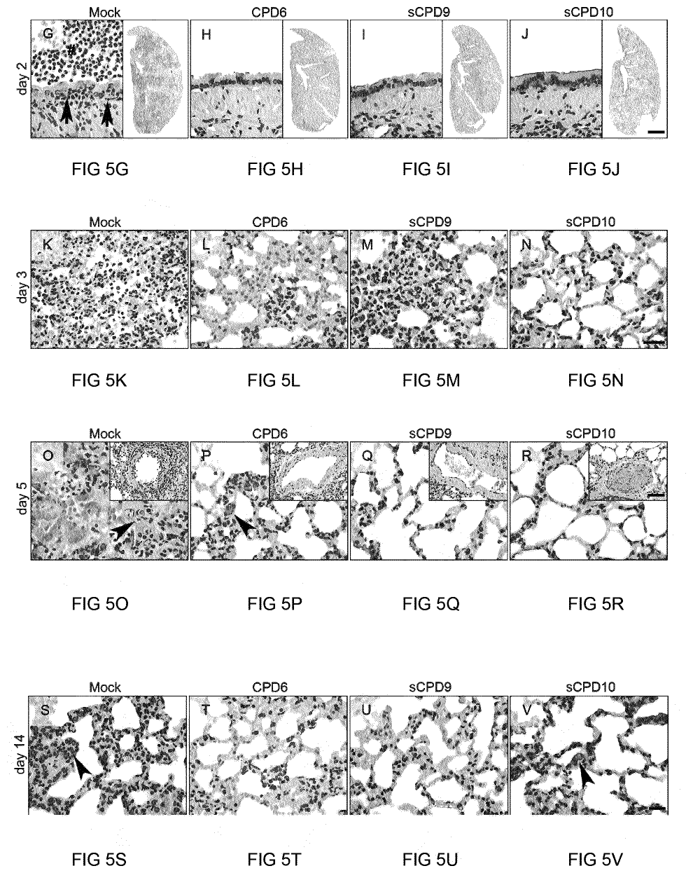

Histopathology of lungs 3 days after vaccination identified marked differences in the intensity and distribution of bronchial and lung lesions between animals infected with some of the recoded viruses and the WT virus (FIGS. 4A to 4N). However, animals within each group developed fairly similar lesions, indicating that lungs of animals were effectively infected and that disease outcomes were homogeneous within each group. Whereas sCPD3 and sCPD4 viruses caused degrees of bronchitis and pneumonia that were relatively similar to the WT virus, sCPD10 virus induced less severe pathology, and infection with CPD6 and sCPD9 viruses resulted in weakest pathology and inflammation, both along the bronchi and in the lung tissue (FIGS. 4A to 4N). A more detailed histopathological examination was done to characterize the protective capacity of the attenuated viruses. For this purpose, hamsters vaccinated with CPD6, sCPD9 or sCPD10 viruses, and challenged 21 days later with WT SARS-CoV-2 were examined following standard evaluation criteria for the COVID-19 Syrian hamster model (Gruber et al., 2020). The results clearly showed that all three attenuated viruses had smaller affected lung areas overall as well as milder degrees of bronchitis, pneumonia and other relevant lesions (FIGS. 5A to 5F and 5G to 5V).

In this context, FIGS. 5A to 5V illustrate the lung histopathology of vaccinated Syrian hamsters on day 2 to 14 after challenge.

FIGS. 5A to 5F show a histopathological evaluation and scoring of lung pathology. Parameters assessed: estimated percentages of affected lung areas (FIG. 5A), degree of bronchitis (FIG. 5B), lung inflammation (FIG. 5C), endothelialitis (FIG. 5D), edema (FIG. 5E) and epithelial hyperplasia (FIG. 5F). Scores and parameters in FIGS. 5B to 5F were classified as absent (0), minimal (1), mild (2), moderate (3), or severe (4). n=3 for each treatment at each time point.

FIGS. 5G to 5V show representative photomicrographs of formalin-fixed, paraffin-embedded, hematoxylin and eosin-stained lung tissues: bronchial epithelium (FIGS. 5G to 5J), air spaces (FIGS. 5K to 5V). Insets in FIGS. 5G to 5J show the distribution of SARS-CoV-2 RNA, visualized by in situ-hybridization in lungs of Syrian hamsters on day 2 after challenge. Red signal: viral RNA; blue: hemalaun counterstain. Insets in FIGS. 5O to 5R show blood vessels affected by endothelialitis on day 5 after challenge. Pathological changes are shown from representative animals that had been either mock-vaccinated (Mock) or vaccinated with CPD6, SCPD9, or sCPD10. Bars: 50 μm (FIGS. 5G to 5V), 1 mm (insets in FIGS. 5G to 5J), 100 μm (insets in FIGS. 5O to 5R).

Due to their particular significance in COVID-19 pneumonia, the inventors additionally scored the intensities of endothelialitis as well as perivascular and alveolar edema, which were also markedly reduced in vaccinated hamsters (FIGS. 5D and 5E). Furthermore, the inventors compared bronchial and alveolar epithelial cell hyperplasia as evidence of tissue regeneration after parenchymal damage. Again, signs indicating the necessity of tissue repair were almost completely missing in hamsters vaccinated with one of the three recoded viruses (FIGS. 5F and 5O to 5R). These observations further supported the notion that all three attenuated viruses had protected hamsters from major tissue damage in a subsequent challenge with the WT virus.

In-situ hybridization was performed to examine the protective effect of vaccination on the virus loads in the lung tissue of hamsters 2 days after challenge, the time when it reaches its peak (Osterrieder et al., 2020). The results clearly demonstrated a complete absence of signals indicative of SARS-CoV-2 RNA throughout the lungs of vaccinated hamsters, whereas a strong positive signal was detected in lungs of mock-vaccinated and challenged hamsters (insets in FIGS. 5G to 5J). It thus seems likely that the drastically reduced lung pathologies in the protected hamsters were due to a markedly diminished initial virus replication in their target cells, which is consistent with the quantity of viral loads in the lungs of vaccinated hamsters as determined by RT-qPCR and virus titration assay (FIGS. 3K to 3P).

### Vaccination Induces High Levels of Neutralizing Antibodies

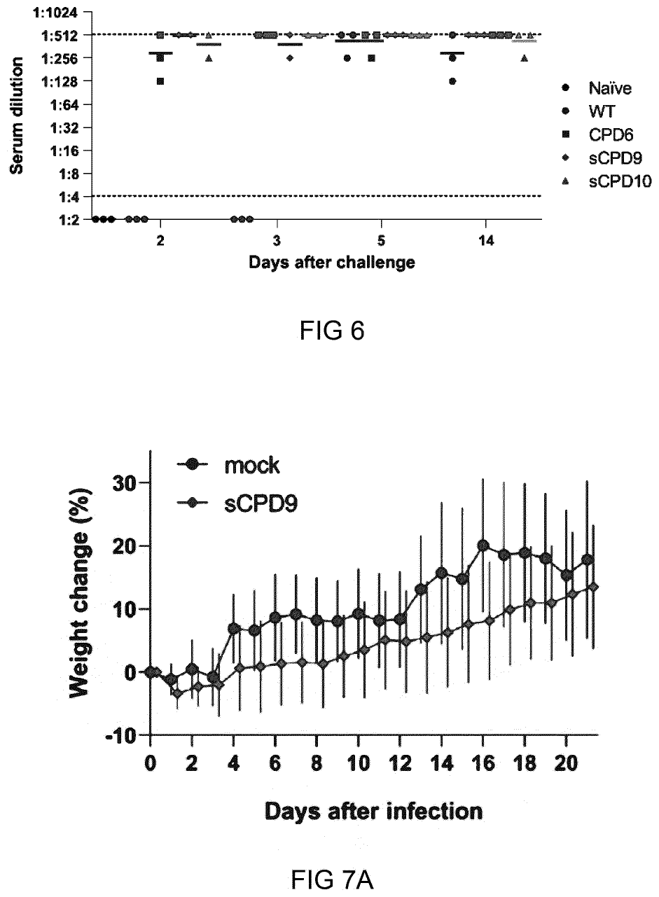

SARS-CoV-2 neutralizing antibodies were quantified in sera from vaccinated hamsters by serum virus neutralization assays (FIG. 6).

In this context, FIG. 6 shows the titers of SARS-CoV-2 neutralizing antibodies in vaccinated Syrian hamsters. SARS-CoV-2 neutralizing antibodies were quantified in sera of mock-vaccinated hamsters and in sera of hamsters vaccinated with CPD6, sCPD9 and sCPD10 after challenge infection with the WT virus. Sera of naïve hamsters were used as a negative control. The dashed lines represents the detection limits of the assay.

The analysis showed that mock-vaccinated hamsters had moderate to high titers of neutralizing antibodies as early as day 5 after challenge infection with WT virus (FIG. 6). This shows that humoral immunity develops a robust response very quickly after infection with the virus. Moreover, neutralizing titers after challenge infection were very high and remarkably consistent in hamsters that were previously vaccinated with CPD6, sCPD9, or sCP10 viruses (FIG. 6). Because the highest dilution the inventors tested was 1:512, the inventors assume individual animals had even higher titers of neutralizing antibodies.

Recoded sCPD9 Virus is Attenuated in Roborovski Dwarf Hamsters

Because the mutant viruses sCPD9 and sCPD10 replicate with delayed kinetics in Vero E6 cells compared with the parental virus and do not produce visible plaques, the inventors initially were unable to produce virus stocks of the two virus mutants with sufficiently high titers. This drawback prevented the inventors from comparing the pathogenicity of these two viruses with that of parental virus in Syrian hamsters that would be infected with a high virus dose (1×10⁵ focus forming units [FFU]/animal). To better assess the degree of attenuation of the lead attenuated vaccine candidate sCPD9, the inventors decided to study its pathogenicity in a rodent species that is highly susceptible to severe COVID-19-like disease, the Roborovski dwarf hamster (Phodopus roborovskii) (Trimpert et al., 2020). When infected with WT SARS-CoV-2, Roborovski dwarf hamsters developed fulminant clinical signs 2 to 4 days after infection, with rapid weight loss, pronounced drop of body temperature, signs of respiratory distress and death (Trimpert et al., 2020).

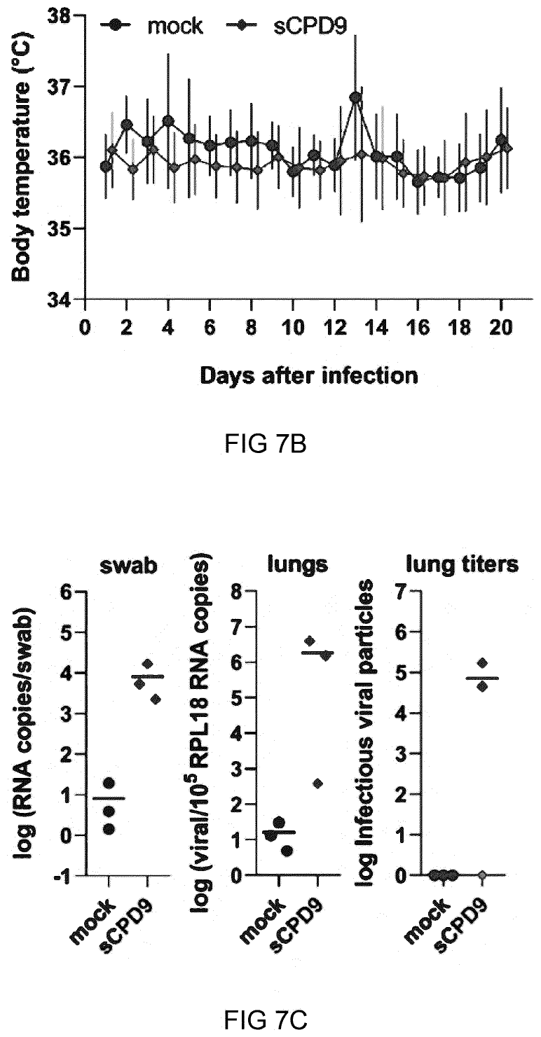

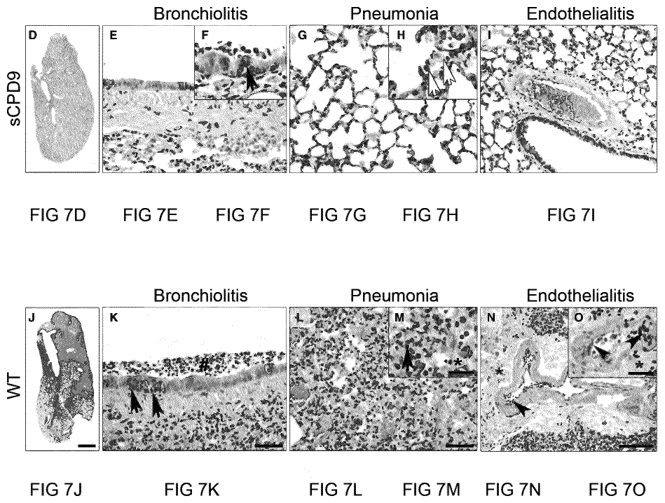

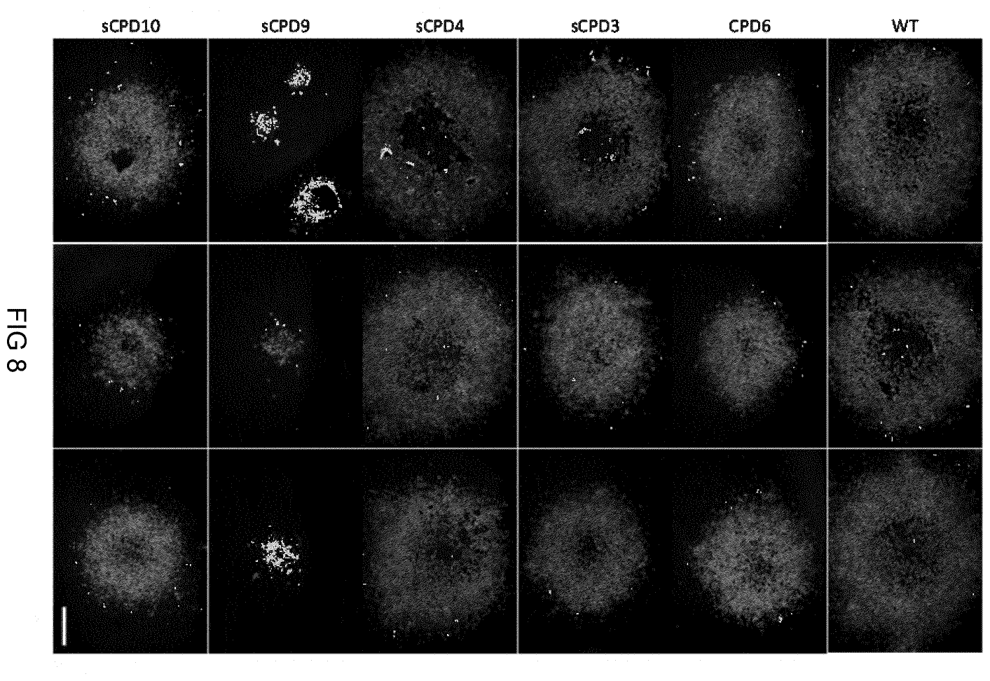

Roborovski dwarf hamsters were randomly assigned to two groups. Twelve hamsters were mock-infected and 30 hamsters were infected with 1×10⁵ FFU of the mutant virus sCPD9. Body weight and body temperature were recorded daily for the duration of the experiment (21 days). On average, the sCPD9-infected hamsters lost only little weight on day 1 (3.4%), but began to gain weight already on day 2 and continued to gain weight steadily until the end of the experiment (FIG. 7A).

In this context, FIGS. 7A to 7O illustrate that recoded SARS-CoV-2 mutant sCPD9s is strongly attenuated in Roborovski dwarf hamsters. FIG. 7A shows weights change of mock-infected (n=12) and sCPD9-infected hamsters (n=30). Data are show as mean±SD.

FIG. 7B shows daily body temperature of mock-infected (n=12) and sCPD9-infected hamsters (n=30). Data are show as mean±SD.

FIG. 7C shows the viral load in the upper (oropharyngeal swab) and lower (lung) airways and infectious virus particles detected in 50 mg of lung tissue on day 3 after infection (lung titers).

FIGS. 7D to 7N illustrate a histopathological evaluation of the infection with sCPD9 (FIG. 7D to 7H) or SARS-CoV-2 variant (WT; FIGS. 7I to 7N) on lung tissues at day 3 after infection. Left lung lobe with mild inflammatory lesions (FIG. 7D). Bronchioli had virtually normal columnar epithelium (FIG. 7E) and only occasional mild bronchiolitis (FIG. 7F); neutrophil (black arrow). The alveolar septa showed mildly increased numbers of macrophages, and few neutrophils (FIG. 7G) with smaller areas of apparent pneumonia with macrophages (white arrows), neutrophils and necrosis of alveolar epithelial cells (FIG. 7H). Normal pulmonary blood vessel (FIG. 7I). Typical lung of hamsters infected with WT virus (FIG. 7J); necrosuppurative bronchiolitis with early hyperplasia of bronchiolar epithelial cells (FIG. 7K); intraluminal cellular debris (hash); infiltrating neutrophils (black arrow). Bronchointerstitial pneumonia (FIG. 7L) with necrosis of alveolar epithelial cells, infiltration by macrophages and neutrophils (FIG. 7M, black arrow) and alveolar edema (FIG. 7M, asterisk). Endothelialitis (arrowhead) with mild to moderate perivascular edema (FIGS. 7N and 7O, asterisk). Scale bars: 1 mm (FIGS. 7D and 7J); 50 μm (FIGS. 7E, 7G, 7K and 7L); 100 μm (FIGS. 71 and 7N); 20 μm (insets FIGS. 7F, 7H, 7M and 7O).

Individual hamsters either lost up to 8.0% weight or gained up to 3.6% weight on day 1 after infection. The mock-infected hamsters did not gain weight during first 3 days after mock infection, but gained weight precipitously on day 4 and steadily thereafter. Daily weight gains were comparable between the two groups, and at the end of this experiment, on day 21, both groups showed comparable weight distribution (FIG. 7A). Note that daily weight averages show greater variation in the case of mock-infected hamsters, probably due to the smaller group size. More importantly, no decrease in body temperature (FIG. 7B) or other clinical signs, such as forced breathing or apathy, which would indicate severe disease were observed in any of the hamsters vaccinated with the sCPD9 virus (Trimpert et al., 2020). Three animals from each group were euthanized on day 3 after infection, and RT-qPCR analysis confirmed that sCPD9 virus replicated in the upper and lower airways of the infected animals (FIG. 7C). Virus reisolation from lung samples was successful in ⅔ of infected sCPD9 animals (FIG. 7B).

Lung Histopathology Confirms Strong Attenuation of sCPD9 Virus in Roborovski Dwarf Hamsters

To assess lung pathology induced by sCPD9 infection, three infected Roborovski dwarf hamsters were euthanized on day 3 after infection. Lungs from these animals and Roborovski dwarf hamsters that were euthanized on day 3 after infection with 1×10⁵ FFU of the WT SARS-CoV-2 variant B.1 in a previous study (Trimpert et al., 2020) were subjected to a detailed histopathological examination (FIGS. 7D to 7O). Although virological parameters confirmed productive viral lower respiratory tract infection in approximately two-thirds of the animals (FIG. 7C), the histologic picture was much milder in these animals compared with WT SARS-CoV-2 infection (FIGS. 7D to 7O). In Roborovski dwarf hamsters infected with sCPD9 virus, mild inflammatory lesions were visible only in small lung areas (FIG. 7D). Of note, pulmonary vessels were devoid of endothelialitis. In sharp contrast, lungs of Roborovski dwarf hamsters infected with the WT SARS-CoV-2 showed extensive inflammation involving up to 70% of the lung tissue (FIG. 7J). Detailed analysis revealed that bronchiolitis, pneumonia, and endothelial inflammation were significantly reduced in sCPD9-infected animals (FIGS. 7E to 7I) compared with the WT-infected animals (FIGS. 7K to 7O).

### DISCUSSION

Several vaccines have demonstrated efficacy in large-scale phase 3 clinical trials and have been approved for human use, and many others are in the final stages of clinical testing (Baden et al., 2021; Dagan et al., 2021; Ella et al., 2021; Emary et al., 2021; Gao et al., 2020; Logunov et al., 2021; Solforosi et al., 2021; Voysey et al., 2021b; Wang et al., 2020; Zhang et al., 2021; Zhu et al., 2020; Zimmer et al., 2021). Despite substantial progress, the majority of the world's population has not been vaccinated. More than 4.4 billion doses of vaccine have been administered globally as of Dec. 12, 2021, yet there is a wide disparity in vaccination progress between high- and low-income countries (Zimmer et al., 2021). As a result, the COVID-19 pandemic continues to disrupt lives and ravage many different regions of the world. To reduce the social and economic burden of the pandemic, it is necessary to develop, manufacture and administer billions of safe, effective, and affordable vaccines.

Among the most widely administered vaccines are mRNA vaccines developed by Pfizer-BioNTech (BNT162b2, or Comirnaty) and Moderna (mRNA-1273), non-replicating adenovirus-vectored vaccines developed by the Gamaleya Research Institute (Sputnik V), University of Oxford—AstraZeneca (AZD1222, Vaxzevria or Covishield) and Janssen-Johnson & Johnson (Ad26.COV2.S), inactivated virus vaccines developed by The Beijing Institute of Biological Products—Sinopharm (BBIBP-CorV), Sinovac Biotech (CoronaVac, or PiCoVacc), Wuhan Institute of Biological Products-Sinopharm (unnamed) and the Indian Council of Medical Research—Bharat Biotech (Covaxin) (Zimmer et al., 2021).

Although no mRNA-based vaccines had been licensed for use in humans previously, they are the most effective and up to now the safest of the licensed COVID-19 vaccines. They showed ˜95% efficacy in preventing symptomatic disease against the original Wuhan virus, including severe disease, and caused almost no serious side effects in various clinical trials and real-world settings (Baden et al., 2021; Haas et al., 2021; Hall et al., 2021). The disadvantage of mRNA vaccines is their high cost and the need for low storage temperatures (Comirnaty), which makes them unaffordable and logistically impractical for many low-income countries.

Vaxzevria is the most widely administered adenovirus-based vaccine (Zimmer et al., 2021). While the efficacy of the vaccine in preventing symptomatic COVID-19 is significantly lower than the mRNA vaccines at ˜75%, the efficacy against severe disease and hospitalization is 100% (Voysey et al., 2021a). The main advantage of this vaccine is its low cost and long stability at standard refrigeration temperatures (2-8° C.). Very rarely, however, the vaccine has led to the formation of diffuse blood clots with dramatically reduced numbers of platelets (Greinacher et al., 2021). As a result, many countries have paused, restricted, or completely discontinued vaccination with adenovirus-based vaccines, as the adenovirus vector is the suspected trigger of the rare but serious side effect.

Preliminary reports from different clinical trials indicate that inactivated virus vaccines are 50-80% effective in preventing COVID-19 caused by WT virus (Mallapaty, 2021). The vaccines appear also very safe, as no serious adverse effects have been observed to date. Inactivated virus vaccines are widely administered in China and few other countries. The World Health Organization has approved BBIBP-CorV and CoronaVac for emergency use, which may increase global trust in these vaccines and alleviate the high demand for vaccines, particularly in lower income countries (Mallapaty, 2021).

With the exception of the inactivated virus vaccines, all currently licensed vaccines are based solely on the viral spike glycoprotein. SARS-CoV-2 is evolving rapidly. During the course of the pandemic, a number of viral variants have accumulated the same or similar mutations in the spike protein through convergent evolution. Recent evidence has shown that some of the acquired mutations can compromise the efficacy of licensed vaccines. Viruses from lineages B.1.1.7 (England), B.1.1.28.1 (Brazil), B.1.1.28.3 (Philippines), B.1.525 (West Africa), and B.1.526 (USA), but especially from lineage B.1.351 and B.1.1.529 (South Africa) have been associated with re-infections and vaccine breakthroughs (Abu-Raddad et al., 2021; Madhi et al., 2021). Widespread vaccination is also expected to increase selective pressure, which could lead to the emergence of SARS-CoV-2 escape variants that cannot efficiently be controlled by vaccination. The emergence of such variants would diminish the efficacy of spike-based vaccines, and necessitate vaccine revisions. In fact, Pfizer, AstraZeneca, and Moderna, three of the largest manufacturers of licensed vaccines, are developing updated vaccines that would target the B.1.351 variant (ClinicalTrials.gov Identifier: NCT04785144) (Zimmer et al., 2021).

An important advantage of live attenuated virus vaccines is that they replicate in vaccinated individuals and thus stimulate an immune response not just against the major surface glycoprotein, but the entire ensemble of virus antigens. Therefore, modified live virus vaccines should theoretically better protect against a wide variety of virus variants, such as the recently emerged B.1.1.7, B.1.351, B.1.1.28.1, B.1.617.2, and B.1.1.529 variants, because they stimulate a broad antibody and cytotoxic repertoire. The preliminary data suggest that a single-dose vaccination with the sCPD9 mutant indeed induces strong protection. In addition, live attenuated virus vaccines can be administered intranasally and thus induce mucosal immune response directly at the site of virus entry. This may offer better protection of target tissues from infection and could further limit disease severity and virus shedding. For similar reasons, the University of Oxford and CanSino Biologics are launching clinical trials to determine whether nasal administration of the Vaxzevria and Ad5-nCOV vaccines, respectively, would increase protection against infection and limit virus transmission (ClinicalTrials.gov Identifiers: NCT04816019, NCT04840992).

Live attenuated virus vaccines also offer many practical advantages that can play an important role in pandemic response in countries where price, distribution, and administration of the vaccine are important factors. The important advantage of the modified live virus vaccine against SARS-CoV-2 is that the production, storage, distribution, and administration of such a vaccine is relatively simple.

Although the viral stocks of sCPD9 and sCPD10 viruses that the inventors prepared initially had relatively low titers (2×10⁵ FFU/ml), the inventors discovered that it was possible to significantly increase the viral yield (1×10⁷ FFU/ml) by optimizing the infection of cells and the timing for virus harvest. SARS-CoV-2 replicates rapidly to relatively high titers in Vero E6 cells, allowing a large number of vaccine doses to be produced within a short time according to standard procedures. In addition, the experiments have shown that vaccination with a relatively low viral dose (2×10⁴ FFU/animal) fully protected Syrian hamsters from WT challenge. It is conceivable that an optimal vaccine dose is also relatively low for humans. This would mean that a large number of vaccine doses could be produced very quickly and inexpensively form a small number of cells. Vero cells are easily cultured and have been approved by many regulatory agencies for the production of various human vaccines, including recent inactivated SARS-CoV-2 vaccines (Ella et al., 2021; Zhang et al., 2021). In addition, the biosafety level for highly attenuated viruses will be downgraded from the current level 3 to level 2, which would further facilitate the production and transport of such vaccines. Frozen or freeze-dried virus is stable—the virus titers drop only moderately after thawing or reconstitution—and the vaccination can be easily administered to patients in the form of nasal drops or sprays. A single-dose vaccine against SARS-CoV-2 offers a strong logistical advantage over a two-dose vaccine for mass vaccination campaigns against the COVID-19 pandemic.

The main disadvantage of live attenuated virus vaccines is the theoretical risk of reversion to virulence. For example, the oral poliovirus vaccine has 51 mutations, but only five of them are attenuating. As a result, this vaccine is prone to reversion, and in rare cases it reverted to virulence and caused paralytic poliomyelitis in vaccinated individuals (Kew et al., 2005). In contrast, recoded viruses contain hundreds of nucleotide mutations, but many of these mutations are attenuating. It is believed that attenuation of CPD viruses occurs through the addition of small defects that each underrepresented “bad” codon pair exerts on gene expression of the recoded gene. Due to the large number of mutations, CPD viruses are generally genetically stable and unlikely to revert to virulence. Nevertheless, it cannot be excluded that vaccination of large numbers of individuals would increase the chances of reversion. Another theoretical possibility is that recoded mutants could adapt to humans and become eventually endemic in the human population.

None of the most advanced vaccine candidates is a modified live or attenuated virus vaccine (WHO). From the three basic types of viral vaccines, modified live virus vaccines are considered the most efficacious vaccines for healthy individuals and generally outperform inactivated, vectored or subunit vaccines, because they generally provoke broad, strong, and durable immune responses, which are qualitatively identical to those induced by a natural infection with circulating virulent strains (Bazin, 2003; Kusters and Almond, 2008; Lauring et al., 2010). Traditionally, modified live virus vaccines have been prepared empirically by the iterative process of serial passage of a virulent virus in cell culture and/or in laboratory animals (Kusters and Almond, 2008; Lauring et al., 2010). The attenuation by CPD represents an alternative to approaches that rely on serendipity as the main attenuating principle. The attenuating mutations are introduced into viral genomes deliberately, based on rational design (Eschke et al., 2018; Groenke et al., 2020; Osterrieder and Kunec, 2018; Trimpert et al., 2021a) In CPD, viral genes are recoded with statistically underrepresented codon pairs, which in turn perturb gene expression and causes attenuation of the recoded virus (Coleman et al., 2008; Groenke et al., 2020). Codon pair bias is a species-specific feature, but phylogenetically closely related species have similar codon pair bias. For example, all mammals have nearly identical codon pair bias (Kunec and Osterrieder, 2016). Thus, mutant viruses carrying genomic sequences that have been codon pair deoptimized on the basis of human codon pair bias should not only be attenuated in human cells, but also in all permissive cells derived from different mammalian species (Groenke et al., 2020). The results are consistent with this assumption, as recoded SARS-CoV-2 mutants were attenuated in cell derived from the African green monkey, but also in two different rodent species.

Because CPD substantially alters genomic sequence, additional phenomena may contribute to the attenuation of recoded viruses. For example, recoding usually increases the number of CpG and UpA dinucleotides in recoded sequences, which then activate innate host defense mechanisms (Odon et al., 2019). In addition, recoding may destroy unknown cis-regulatory regions, thereby dampening viral replication (Song et al., 2012). Alternative attenuation strategies based on large-scale recoding of viral genomes take advantage of this knowledge and intentionally increase the number of CpG or UpA dinucleotides, rare or “near-stop” codons in viral genomes, as these (interrelated) modifications often lead to the generation of replication-competent, but severely attenuated viruses (Lauring et al., 2012; Osterrieder and Kunec, 2018).

The data presented here demonstrate that a single-dose vaccination with CPD6, sCPD9 or sCPD10 viruses protects vaccinated hamsters from developing disease after challenge infection with virulent SARS-CoV-2 (FIGS. 3A to 7O). This was evidenced by histological evaluation of lung pathology, which showed virtually no correlate of damage induced by challenge virus replication after vaccination with CPD6, sCPD9 and sCPD10. Pathological lesions could be detected only in a small part of the lung tissue and generally revealed only mild bronchitis, pneumonia and edema (FIGS. 5A to 5C). Similarly, epithelial hyperplasia was very limited in vaccinated animals at later times after challenge. This absence of cellular repair and regeneration in the lung supports the conclusion that all three vaccine candidates had protected the hamsters from relevant tissue damage caused by challenge virus infection (FIG. 5F).

The amount of viral RNA detected in the lungs of challenged animals was very low on days 2, 3 and 5 after the challenge, and, more importantly, the inventors could not isolate replicating virus from the lungs of the animals after challenge infection (FIGS. 3K to 3P). The data thus show that, although recoded viruses were unable to protect the upper respiratory tract from reinfection with the virulent WT virus, they did protect the lower respiratory tract very well from infection.

The inventors surmise that a single-dose vaccination with a live modified attenuated virus should provide protective immunity against infection with pathogenic SARS-CoV-2, as the inventors were unable to detect infectious virus particles in the lungs of any of the vaccinated animals. CPD6-, sCPD9- and sCPD10-vaccinated animals did not lose any weight after challenge, and histopathological evaluation of the lungs shows clear protective effect of all three vaccine candidates tested. The same data also suggest that a two-dose regimen should also be possible if the development of a more permanent immunity were desirable.

Because sCPD9 was the most attenuated mutant virus examined, but still induced strong protective immunity to disease induced by virulent virus, the inventors decided to investigate the safety of this virus in a recently identified non-transgenic rodent model of COVID-19, the Roborovski dwarf hamster, which mirrors severe human cases of COVID-19 and thus provides unprecedented sensitivity for preclinical testing of vaccine candidates (Trimpert et al., 2020). This experiment proved that the sCPD9 virus is highly attenuated, as the virus did not induce signs of disease in any of the 30 Roborovski dwarf hamsters and caused only minimal pathological changes in the lungs of infected animals.

Further studies will have to address the durability of protective immunity in vaccinated animals. A known limitation of this study is that the inventors have not evaluated how well the live attenuated viruses can transmit from infected animals to sentinels. While Syrian hamsters readily transmit virulent SARS-CoV-2 to co-housed, uninfected contact animals in the first days after infection (Sia et al., 2020), the inventors did not observe any clinical and only limited histological signs of disease in naturally infected contact animals. In the inventors' opinion, the Syrian hamster model has limited utility for studying the spread of attenuated viruses and the disease outcomes of infection by natural transmission. Therefore, for the purpose of this study, the inventors focused on characterizing attenuation of the lead vaccine candidate sCPD9 by experimental high-dose infection of a species that is highly susceptible to severe COVID-19-like disease. In addition, the inventors are preparing experiments in ferrets, a well-described model for studying the spread of respiratory viruses (Kutter et al., 2021), to answer questions related to spread of recoded viruses as well as the efficacy against recent SARS-CoV-2 variants of concern in the near future.

In summary, of all the engineered live attenuated viruses tested, sCPD9 showed the best balance between attenuation, safety, immunogenicity and protective efficacy. The inventors believe that the safety, immunogenicity and vaccine efficacy of this candidate should be investigated in different animals, non-human primates and ultimately humans.

### EXPERIMENTAL MODEL AND SUBJECT DETAILS

### Cells and Viruses

Vero E6 (ATCC CRL-1586) and BHK-21 (ATCC CCL-10) cells were grown in minimal essential medium (MEM) containing 10% fetal bovine serum (PAN Biotech), 100 IU/ml penicillin G and 100 μg/ml streptomycin (Carl Roth) at 37° C. and 5% CO₂. Parental and mutant SARS-CoV-2 were grown in Vero E6 cells. The ancestral SARS-CoV-2 variant B.1 (SARS-CoV-2/München-1.1/2020/929) (Wölfel et al., 2020) was used as a challenge virus. Virus stocks were stored at −80° C. prior to experimental infections.

### Ethics Statement

In vitro and animal work was done under biosafety conditions in the BSL-3 facility at the Institut für Virologie, Freie Universität Berlin, Germany. All animal experiments were approved by the Landesamt für Gesundheit und Soziales in Berlin, Germany (permit number 0086/20) and done in compliance with relevant national and international guidelines for care and humane use of animals.

### Animal Husbandry

The animal experiments were done in a certified BSL-3 facility. Six-week-old Syrian hamsters (Mesocricetus auratus; breed RjHan: AURA) were purchased form Janvier Labs, and they were kept in individually ventilated cages (IVCs; Tecniplast), which were equipped with generous enrichment (Carfil). Five- to seven-week-old Roborovski dwarf hamsters (Phodopus roborovskii) were purchased through the German pet trade from a single breeding facility. They were housed in groups of six animals in IVCs. The animals had unrestricted access to food and water and were allowed to acclimate to the conditions for seven days prior to infection. During both experiments, cage temperatures ranged from 22 to 24° C. and relative humidity ranged from 40 to 55%.

### Infection Experiments

To study the attenuation and vaccine protection of recoded virus mutants in Syrian hamsters, male and female hamsters were randomly assigned into groups of 15 animals, with each group containing 7 or 8 males and female animals. In the first trial, hamsters were either mock-vaccinated or vaccinated with CPD6, sCPD3 or sCPD4 viruses. Hamsters were mock-vaccinated with 60 μl medium from uninfected Vero E6 cells or vaccinated with 1×10⁵ FFU of the mutant virus in 60 μl by intranasal instillation under anesthesia (Osterrieder et al., 2020). In the second trial, hamsters were either mock-vaccinated or vaccinated with sCPD9 or sCPD10 mutant viruses. The vaccination was done as described above, but vaccinated hamsters received only 1×10⁴ FFU of the mutant virus. Twenty-one days after vaccination hamsters were challenged by intranasal instillation with 1×10⁵ FFU of the challenge virus (variant B.1, strain SARS-CoV-2/München-1.1/2020/929) in 60 μl. During the experiment, all hamsters were monitored twice daily for the clinical signs of disease, and body temperatures and body weight were recorded. Hamsters that had a body weight loss of more than 10% weight over a 72-h period were euthanized in compliance with the animal use protocol. On day 3, and on days 23, 24, 26 and 35 after infection (2, 3, 5 and 14 days after challenge) three hamsters of each group were euthanized (Nakamura et al., 2017). Blood, oropharyngeal swabs and lungs (left and right) were collected for virus titrations, RT-qPCR and/or histopathological examinations. All organs were preserved in 4% formalin for subsequent in-depth histopathological investigations.

To assess the pathogenicity of the recoded mutant virus sCPD9 in the Roborovski dwarf hamster, animals were randomly assigned into two groups, with 60% of the animals in each group being female. Twelve hamsters were mock-infected with cell culture medium and 30 hamsters were infected with 1×10⁵ FFU of the mutant virus sCPD9. Anesthetized hamsters were infected as described above, except that the inoculum was 20 μL. Infected hamsters were monitored twice daily for clinical signs of infection. Body weight and temperature was recorded daily. Three hamsters were sacrificed on day 3 after infection to determine virological and histological parameters of infection on day 3 after vaccination. Infected hamsters were monitored twice daily for clinical signs of infection, and body weight and temperature were recorded once daily. Three hamsters from each group were euthanized on the third day after infection to determine virologic and histologic parameters of infection by molecular and virologic assays as described above.

### Method Details

### Recoding of the SARS-CoV-2 Genome

Because the inventors planned to make SARS-CoV-2 mutants via TAR cloning, the inventors designed recoded SARS-CoV-2 fragments to be fully compatible with the available reverse genetics system of SARS-CoV-2 (Thi Nhu Thao et al., 2020). All indicated sequence positions in the SARS-CoV-2 genome correspond to the SARS-CoV-2 reference sequence NC_045512.2.

The coding sequences of SARS-CoV-2 were recoded by CPD (Coleman et al., 2008). CPD rearranges the positions of synonymous codons in recoded viral sequences, thereby creating codon-pair combinations that are statistically underrepresented in protein-coding sequences of the virus host. Because the goal was to generate SARS-CoV-2 mutants that should be attenuated for humans, the inventors recoded SARS-CoV-2 sequences using the codon pair score (CPS) calculated for human protein coding genes (Groenke et al., 2020).

The subgenomic fragments were recoded individually, only using the existing codons present in each respective fragment. The creation of novel transcription regulatory sequence (TRS; ACGAAC) and EagI restriction enzyme sites (CGGCCG) in recoded sequences was disallowed. The generation of novel TRS sites was disallowed not to cause aberrant transcription and EagI restriction enzyme sites were reserved for the release of subgenomic fragments from plasmids.

Two genomic sequences containing essential cis-acting RNA elements were excluded from the recoding. The first sequence that was omitted from recoding was the 503 bp sequence (NC_045512.2; nucleotides 13,451-13,953) containing the frameshift stimulation element (FSE). In the SARS-CoV-2 genome, the FSE is present between the overlapping genes ORF1a and ORF1b and is essential for −1 programmed ribosomal frameshifting. The 106 bp sequence containing the transcription regulatory sequence (TRS) of the Spike gene (NC_045512.2; nucleotides 21,505-21,610) was also omitted from the recoding. The TRS of the spike gene is located at the end of the ORF1b of the SARS-CoV-2 genome. The non-recoded sequences are carried by fragments 6 and 9 of the yeast-based SARS-CoV-2 reverse genetics system, respectively (FIGS. 1A to 1D). The sequences of the recoded DNA fragments have been deposited to the NCBI GenBank database (MZ064531-MZ064546).

### Recovery of Mutant SARS-CoV-2

Infectious viruses were recovered either directly from BAC/YAC SARS-CoV-2 DNA in Vero E6 cells, or from in vitro transcribed viral RNA in BHK-21 cells. To rescue virus progeny from DNA, the immediate-early promoter of cytomegalovirus was inserted upstream of the SARS-CoV-2 genome in BAC/YAC SARS-CoV-2 clones, exactly as described previously (Almazan et al., 2000; Noskov et al., 2002). To promote the recovery of infectious viruses and to monitor transfection efficiency the inventors constructed a dual expression plasmid pVITRO2-EGFP-N which expresses EGFP and SARS-CoV-2 nucleocapsid protein from two different eukaryotic promoters. To recover infectious virus Vero E6 cells were grown to 95% confluence in a T25 cell culture flask and transfected with 4 μg of BAC/YAC DNA and 1 μg pVITRO2-EGFP-N plasmid using the Xfect single shots transfection reagent (Takara Bio Inc.)

To recover infectious viruses in BHK-21 cells, viral RNA was produced by in vitro transcription using the mMESSAGE mMACHINE™ T7 transcription kit (Thermo Fisher Scientific). Ten μg of in vitro transcribed viral RNA and 1 μg of pVitro2-EGFP-N plasmid were mixed with 1×10⁶ BHK-21 cells resuspended in 100 μl OptiPro medium (Thermo Fisher Scientific) and electroporated in a 2 mm gap cuvette by applying one pulse of 140 V for 25 ms in a Gene Pulser Xcell (Bio-Rad Laboratories). Electroporated BHK-21 cells were co-cultured with susceptible Vero E6. Recovered progeny virus was collected from cell culture medium and grown on Vero E6 cells.

### Multi-Step Growth Kinetics

To assess the kinetics of virus growth, Vero E6 cells grown in 6-well plates were infected with the parental or recoded viruses at a multiplicity of infection (MOI) of 0.01. After 2 h incubation, the viral inoculum was removed and replaced by growth medium. Six, 12, 24, 48 and 72 hours after infection, cell culture medium was collected and virus titers were determined by focus forming assays.

### Virus Titrations, Indirect Immunofluorescence and Focus-Forming Assay

To determine virus titers as focus-forming units (FFU) and foci sizes, Vero E6 cells grown in 12-well plates were infected with 100 μl of serial 10-fold dilutions of virus. To determine virus titers in the lung, 50 mg lung tissue was first homogenized with a bead mill (Analytic Jena) and the homogenate was serially diluted. After 2 h of incubation, the viral inoculum was removed and cells were overlaid with MEM containing 0.6% microcrystalline cellulose Avicel (FMC BioPolymer). Forty-eight hours after infection cells were fixed with 4% formalin, permeabilized with 0.1% Triton-X 100, and blocked with 3% BSA in PBS. Cells were then incubated with a monoclonal mouse anti-SARS-CoV-2 nucleocapsid antibody (provided by Sven Reiche, Friedrich Loeffler Institute, Riems, Germany) for 1 h, and then with goat anti-mouse IgG-Alexa Fluor 568 secondary antibody (Invitrogen) for 45 min. To determine the cell-to-cell spread of mutant viruses in cells, images of 30 randomly selected foci of infected cells were taken at 50-fold magnification using an inverted fluorescence microscope (Axiovert S100, Zeiss). The foci areas were measured using ImageJ software (Schneider et al., 2012), from which the diameters were calculated.

### Histopathology and In Situ-Hybridization

For histopathology and localization of viral RNA by in situ-hybridization (ISH), the left lung lobe was carefully removed and immersion fixed in buffered 4% formalin, pH 7.0, for 48 h. Lungs were embedded in paraffin and cut at 2 μm thickness. Sections were stained with hematoxylin and eosin (HE) and periodic acid-Schiff (PAS) reaction followed by blinded microscopic evaluation by board certified veterinary pathologists (K. D., A. D. G.) (Gruber et al., 2020). For ISH, the ViewRNA™ ISH Tissue Assay Kit (Invitrogen by Thermo Fisher Scientific, Darmstadt, Germany) was used following the manufacturer's instructions with minor adjustments. Probe for the detection of the N gene RNA of SARS-CoV-2 (NCBI database NC_045512.2, nucleotides 28,274 to 29,533, assay ID: VPNKRHM) was employed. Lung sections of 2 μm thickness mounted on adhesive glass slides were dewaxed in xylol and dehydrated in graded ethanol. Tissues were incubated at 95° C. for 10 min and subsequently protease digested for 20 min. Sections were fixed with 4% paraformaldehyde dissolved in phosphate buffered saline (PBS) and hybridized with the probes. Amplifier and label probe hybridizations were performed according to the manufacturer's instructions using fast red as chromogen. Sections were counterstained with hematoxylin for 45 s, washed in tap water for 5 min, and mounted with Roti®-Mount Fluor-Care DAPI (4,6-diamidino-2-phenylindole; Carl Roth). An irrelevant probe for the detection of streptococcal pneumolysin was used as a control for sequence-specific binding. HE and PAS-stained and ISH slides were analyzed and photographed using an Olympus BX41 microscope with a DP80 Microscope Digital Camera and the cellSens™ Imaging Software, Version 1.18 (Olympus Corporation, Münster, Germany). For overviews with lower magnification, slides were automatically digitized using the Aperio CS2 slide scanner (Leica Biosystems Imaging Inc., Vista, CA, USA) and image files were generated using the Image Scope Software (Leica Biosystems Imaging Inc.).

### RNA Isolation and RT-qPCR

RNA was extracted from 25 mg lung homogenates and oropharyngeal swabs using the innuPREP Virus RNA kit (Analytik Jena AG). SARS-CoV-2 RNA copies were quantified using the NEB Luna Universal Probe One-Step RT-qPCR kit (New England Biolabs) on a StepOnePlus RealTime PCR System (Thermo Fisher Scientific) as previously described (Corman et al., 2020).

### Serum Virus Neutralization Assay

Neutralization assay was performed by two-fold serial dilutions (1:4 to 1:512) of complement inactivated (56° C., 2 h) hamster serum plated on sub-confluent monolayers of Vero E6 cells in 96 well plates. 50 FFU SARS-CoV-2 were added per well and incubated for 72 h at 37° C., fixed with 4% formalin for 24 h and stained with crystal violet (0.75% aqueous solution). Serum neutralization was considered effective in wells that did not show any cytopathic effect, the last effective dilution was counted.

### Western Blotting

Confluent Vero E6 cells grown in 6-well plates were infected with an MOI of 0.1. After 48 h, infected cells were frozen at −80° C., resuspended in cell culture supernatant and lysed in 2× Laemmli buffer containing protease inhibitors (Roche). Cell lysates were incubated at 95° C. for 30 min, and proteins were separated by sodium dodecyl sulfate (SDS)-polyacrylamide gel electrophoresis (PAGE) under reducing conditions. Proteins were transferred to PVDF membranes by semi-dry blotting. Membranes were blocked for 1 h at room temperature in 3% milk in PBS containing 0.05% Tween-20 and then incubated with the primary antibody for 16 h at 4° C. Nucleoprotein was detected with a mouse monoclonal anti-N antibody (see above) and spike protein with a mouse monoclonal anti-S2 antibody (1A9, GeneTex). Horseradish peroxidase-conjugated anti-mouse IgG (1:2000, Merck) was used as a secondary antibody. Antibody binding was visualized using chemiluminescent Amersham ECL prime western blotting detection reagent (Thermo Fisher Scientific).

### Quantification and Statistical Analysis

Statistical tests were performed using GraphPad/Prism. Statistical details of experiments can be found in the figure legends. Exact values of n and statistical tests are indicated in figure legends. P values are indicated in Figures. Data are presented as mean±SD.

### Second Exemplary Embodiment

### Results

As explained in connection with the first exemplary embodiment, the inventors constructed a series of live attenuated SARS-CoV-2 vaccine candidates by large-scale recoding of the SARS-CoV-2 genome (Trimpert et al., 2021b). The SARS-CoV-2 genome was modified by codon pair deoptimization (CPD), an approach that has resulted in rapid and efficient attenuation of a variety of RNA (Coleman et al., 2008; Groenke et al., 2020) and DNA viruses (Eschke et al., 2018; Khedkar et al., 2018). CPD is based on the observation that some codon pairs occur in protein-coding sequences significantly less or more frequently than would be statistically expected (Gutman et al., 1989). In CPD, viral genes are recoded to contain an increased number of codon pairs that are statistically underrepresented (suboptimal) in the host and are designed to lead to attenuation of the recoded viruses (Coleman et al., 2008; Groenke et al., 2020; Eschke et al., 2018). In the process of CPD recoding, the positions of synonymous codons in the recoded sequence are exchanged in such a way that the frequency of underrepresented codon pairs increases. Because CPD exchanges only the positions of synonymous codons in recoded sequences, the recoded, live attenuated virus contains exactly the same antigens as the pathogenic parent. The introduced genetic changes reduce protein production from the recoded genes and also the replication fitness of the mutant virus (Groenke et al., 2020). The conserved antigenic identity and the remaining replicative potential enable the recoded attenuated virus to fully engage the immune system of the host and provoke strong immune responses (Coleman et al., 2008; Groenke et al., 2020; Eschke et al., 2018).

In the first exemplary embodiment and a previous study, the inventors have evaluated live attenuated virus candidates and have shown that some of them are strongly attenuated, induce strong immune responses, and protect Syrian hamsters from a challenge with ancestral wild-type (WT) SARS-CoV-2 (Trimpert et al., 2021b). In that study, the inventors also showed that the lead live attenuated virus candidate sCPD9 is strongly attenuated in the Roborovski dwarf hamster (Phodopus roborovskii), which is highly susceptible to severe COVID-19-like disease (Trimpert et al., 2020). Despite the high susceptibility of this hamster species, sCPD9 did not cause clinical disease or considerable lung pathology (Trimpert et al., 2021b).

The sCPD9 virus is also highly attenuated in vitro, where it grows to 100-fold lower titers than WT virus under normal conditions on Vero E6 cells (Trimpert et al., 2021b). Nonetheless, it is possible to produce virus stocks with high titers (1×10⁷ infectious virus particles/ml) using the procedure described in Materials and Methods. In this second embodiment, the inventors assessed immunogenicity and protective efficacy of the sCPD9 virus against the ancestral B.1 and two VOCs, B.1.1.7 and B.1.351.

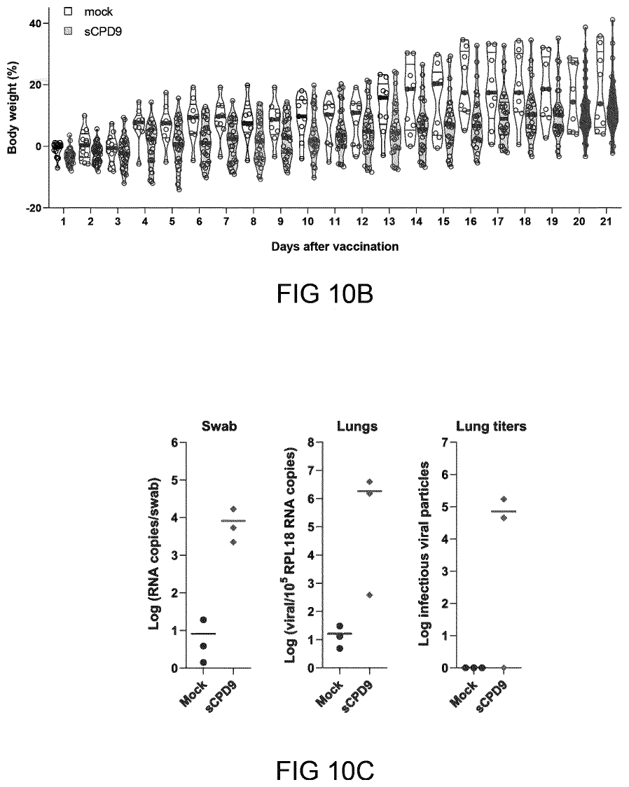

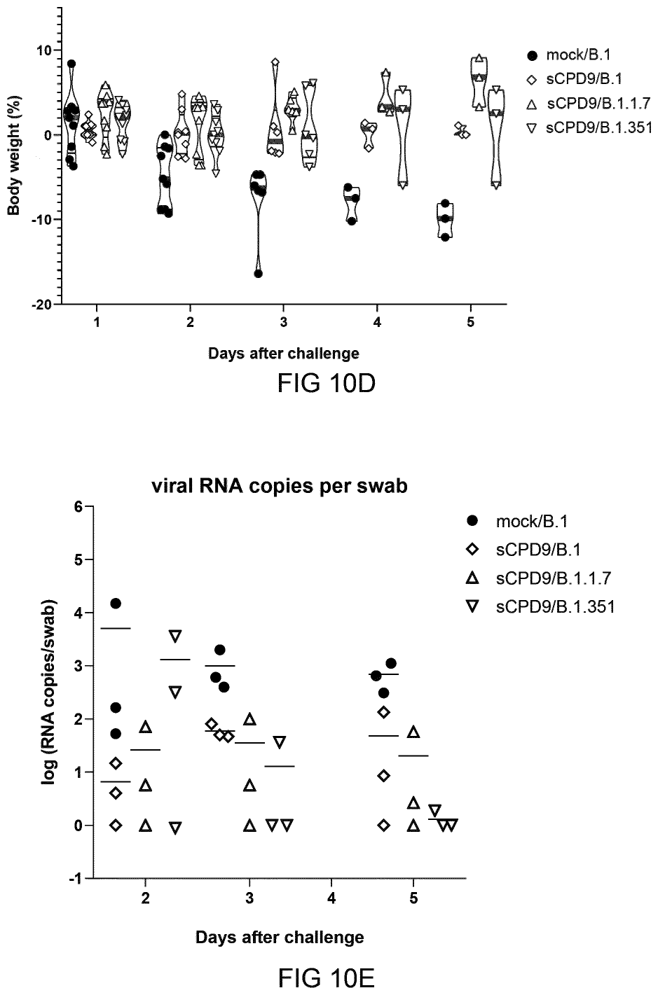

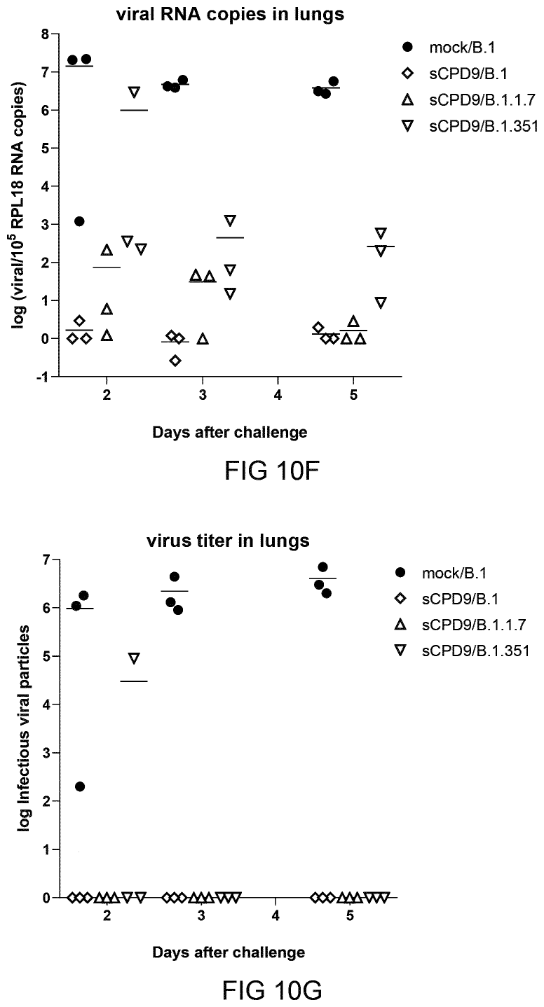

FIG. 10 shows that live attenuated virus vaccine candidate sCPD9s is strongly attenuated and protects Roborovski dwarf hamsters from challenge with B.1, B.1.1.7, and B.1.351 viruses. FIG. 10B shows body weight change of Roborovski dwarf hamsters after vaccination. The thick black lines show median, and the thin lines show the quartiles. FIG. 10C illustrates the viral load in the upper (oropharyngeal swab) and lower (lungs) respiratory tract and the amount of infectious virus detected in 50 mg of lung tissue (lung titers) on day 3 after vaccination. FIG. 10D shows the body weight change of Roborovski dwarf hamsters after challenge with pathogenic B.1, B.1.1.7, or B.1.351 viruses. FIGS. 10E and 10F show the viral load in the upper (FIG. 10E) and lower (FIG. 10F) respiratory tracts of animals on days 2, 3, 5, and 5 after challenge. FIG. 10G shows the amount of infectious virus detected in 50 mg of lung tissue on days 2, 3, and 5 after challenge.

### Vaccination

Hamsters were randomly assigned to two groups of 12 and 30 animals. On day 0, 12 hamsters were mock-vaccinated and 30 hamsters were vaccinated with 1×10⁵ focus-forming units (FFUs) of the sCPD9 virus by intranasal instillation. On day 21, hamsters were challenged with 1×10⁵ plaque-forming units (PFUs) of variants B.1, B.1.1.7, or B.1.351. Body weights and clinical signs were recorded daily for the duration of the experiment (26 days). Three animals of each group per time point were euthanized on day 3 after vaccination and on days 2, 3, and 5 after challenge to determine the degree of virus replication in various organs and to assess lung pathology (Trimpert et al., 2021b).

On average, sCPD9-vaccinated hamsters gained slightly less weight than mock-vaccinated hamsters in the 21 days after vaccination, but differences were not significant (FIG. 10B). No adverse reactions or clinical signs, such as forced breathing or apathy, that would indicate severe disease were observed in any of the mock- or sCPD9-vaccinated hamsters. Attenuated sCPD9 virus replicated efficiently in the upper and lower respiratory tract of sCPD9-vaccinated hamsters on day 3 after vaccination (FIG. 10C). In addition, the inventors detected infectious viruses in the lungs of two-thirds of sCPD9-vaccinated animals on day 3 after vaccination (FIG. 10C). Histopathological examination of lungs of sCPD9-vaccinated hamsters on day 3 after vaccination showed that sCPD9 is strongly attenuated, as only few mild inflammatory lesions could be detected in the lungs of vaccinated animals (Trimpert et al., 2021b).

### Challenge Infection

Vaccination with the sCPD9 virus induced robust protective immunity against challenge with all three SARS-CoV-2 variants. None of the vaccinated hamsters showed clinical signs of disease, and none of the challenged groups lost weight after challenge (FIG. 10D). In contrast, hamsters of the mock-vaccinated group showed typical signs of severe COVID-19-like disease, began to lose weight on day 2 after challenge, and continued to lose weight until the end of the study (day 5 after challenge) (FIG. 10D).

Virological assessments of the challenge infection confirmed virus replication in the upper airways of infected animals on days 2, 3, and 5 after challenge (FIG. 10E). The detection of viral RNA in the upper respiratory tract of many vaccinated animals suggests that the virus remains capable of establishing local infection, regardless of the variant used for challenge. Nevertheless, vaccination drastically reduced the amount of viral genomic RNA in the lower respiratory tract of challenge-infected hamsters (FIG. 10F). Vaccinated hamsters had distinctly lower viral loads than control animals on all days examined. The viral load in the control group was one- to six-fold higher on day 2 and four- to five-fold higher on days 3 and 5 after challenge (FIG. 10F). As expected, variants replicated with unequal efficiency in the lungs of challenged hamsters. Hamsters infected with the ancestral SARS-CoV-2 variant B.1, which is antigenically identical with the sCPD9 virus, produced the lowest viral loads, whereas those infected with B.1.351 virus had the highest viral loads.

To further evaluate the immunity established by vaccination, the inventors attempted to isolate the challenge virus from lungs of animals in cultured Vero E6 cells. This evaluation showed sterilizing immunity in the lower airways of all but one subject. While the inventors failed to isolate B.1 and B.1.1.7 viruses, the inventors detected infectious, replication-competent virus in a single hamster (one of three hamsters) in the B.1.351 group, but only shortly after challenge infection, on day 2 after challenge (FIG. 10G).

Histopathology after Challenge

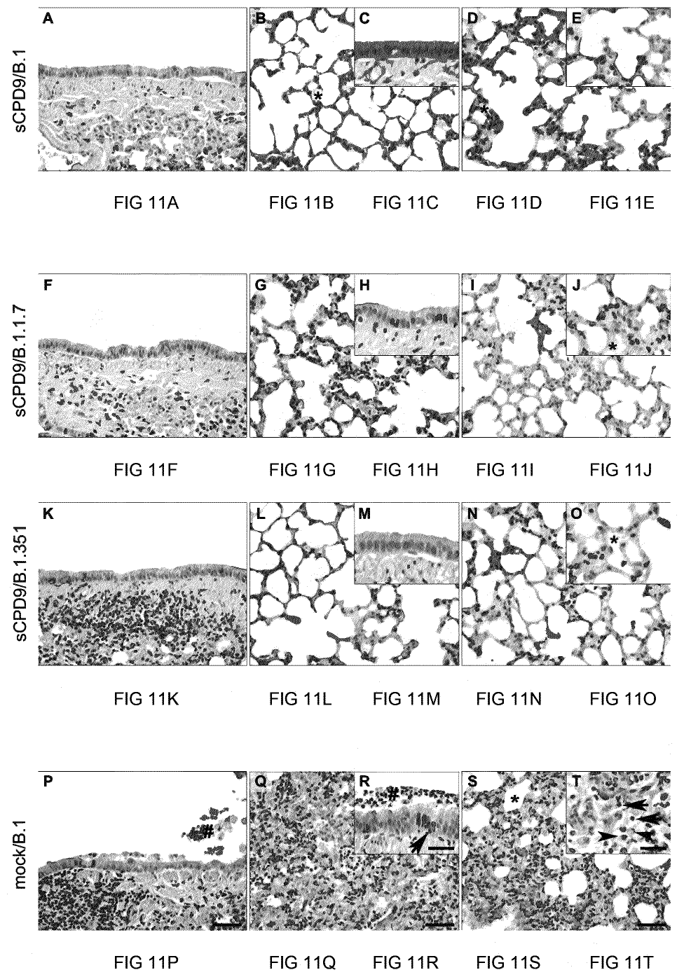

To determine the protective capacity of vaccination with the sCPD9 virus against COVID-19-like disease caused by three different SARS-CoV-2 variants, the lungs of challenged hamsters were subjected to detailed histopathological examination (FIGS. 11A to 11T).

In this context, FIGS. 11A to 11T illustrate the pulmonary histopathology of mock/sCPD9-vaccinated and challenged Roborovski dwarf hamsters on days 2 (left column), 3 (middle column), or 5 (right column) after challenge.

FIGS. 11A to 11T show representative photomicrographs of hematoxylin and eosin (H & E)-stained, formalin-fixed, and paraffin-embedded lung tissue of infected hamsters. sCPD9-vaccinated hamsters were challenged with B.1, B.1.1.7, or B.1.351 virus on day 21 after vaccination. The lungs of sCPD9-vaccinated hamsters that were challenge-infected with three different SARS-CoV-2 variants showed very similar pulmonary morphology. Bronchioles had regular, columnar bronchiolar epithelium on days 2 and 3 after challenge (FIGS. 11A, 11C, 11F, 11H, 11K, and 11M). Alveoli on days 3 (FIGS. 11B, 11G, and 11L) and 5 after challenge (FIGS. 11D, 11E, 11I, 11J, 11N, and 110) showed mild to moderate infiltration by macrophages and few neutrophils with scattered mild alveolar edema (asterisk).

In contrast, mock-vaccinated hamsters challenge-infected with the ancestral SARS-CoV-2 variant B.1 displayed typical lesions of COVID-19 at all times tested (FIGS. 11P to 11T). After 2 days, the bronchiolar epithelium was flattened due to necrosis of bronchiolar epithelial cells and necrotic cellular debris, and degenerate neutrophils and macrophages were present in the bronchiolar lumen (hash) (P). In the course of disease, there was moderate to severe bronchi interstitial pneumonia with necrosis of alveolar epithelial cells and massive infiltration by macrophages and neutrophils in the alveolar septa and alveolar spaces (FIG. 11Q). Similarly, bronchiolar epithelium was infiltrated by an increasing number of neutrophils (arrow) intraluminal cellular debris was accumulating (hash) (FIG. 11R). On day 5 after challenge, lungs showed predominantly interstitial pneumonia with remnants of alveolar edema fluid (asterisk) (FIG. 11S), neutrophils (FIG. 11T, arrow), and an increasing number of macrophages with prominently foamy cytoplasm (FIG. 11T, arrowhead). The scale bars represent 50 μm (FIGS. 11A, 11B, 11D, 11F, 11G, 11I, 11K, 11L, 11N, 11P, 11Q, and 11S) and 20 μm (FIGS. 11C, 11E, 11H, 11J, 11M, 110, 11R, and 11T).

The examination revealed that all sCPD9-vaccinated hamsters were fully protected against SARS-CoV-2-induced inflammatory damage to the lung, irrespective of the variant used for challenge infection (FIGS. 11A to 11T). In contrast, mock-vaccinated and B.1-infected hamsters developed severe lung pathology typical of experimental SARS-CoV-2 infection (Trimpert et al., 2020). These observations support the conclusion that vaccination with sCPD9 virus protected hamsters from major tissue damage and provided complete protection against COVID-19-like disease after challenge infection with three different SARS-CoV-2 variants.

Serum Neutralization after Challenge Infection

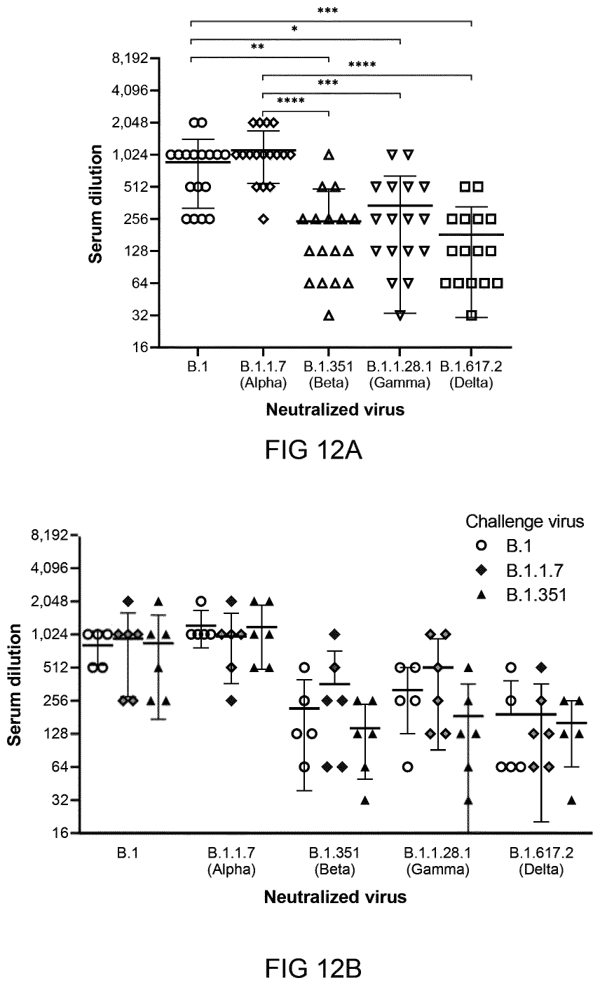

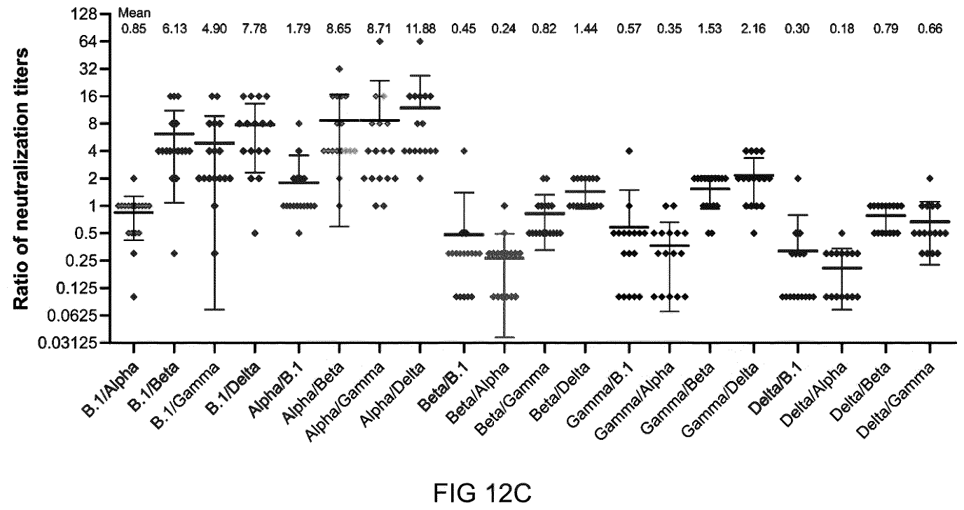

The inventors quantified the capacity of 17 hamster sera that were collected on days 2 and 3 after challenge to neutralize SARS-CoV-2 variants B.1, B.1.1.7, B.1.351, B.1.128.1, and B.1.617.2 by a microneutralization assay (FIG. 12A).

In this context, FIG. 12 illustrates the sensitivity of SARS-CoV-2 variants B.1, B.1.1.7 (Alpha), B.1.351 (Beta), B.1.128.1 (Gamma), and B.1.617.2 (Delta) to neutralization by antibodies in sera of sCPD9-vaccinated Roborovski dwarf hamsters.

FIG. 12A shows neutralizing antibody titers of hamster sera collected on days 2 and 3 after challenge. Geometric mean and SD of the respective group titers are shown. The Kruskal-Wallis test with Dunn's post hoc test showed that hamster sera neutralized the virus variants B.1.351, B.1.128.1, and B.1.617.2 significantly less efficiently than variants B.1 or B.1.1.7 (*P=0.0264, **P=0.0012, ***P<0.001, and ****P<0.0001).

FIG. 12B shows titers of neutralizing antibodies that are plotted as a function of challenge virus. FIG. 12C shows ratios of serum neutralization titers plotted against different virus variants. The ratios show to which extent each hamster serum neutralizes different virus variants. The ratios of the neutralization titers of individual sera, the geometric mean, and the SD are shown. The geometric mean is also displayed as a number in the upper part of the diagram.

Sera from mock-vaccinated hamsters had no neutralization activity against all SARS-CoV-2 variants tested. Sera from sCPD9-vaccinated hamsters had strong neutralizing activity against the B.1 and B.1.1.7 variants, with geometric mean titers of 873 and 1129, respectively, but lower neutralizing activity against the B.1.351, B.1.128.1, or B.1.617.2 variants, with geometric mean titers of 243, 341, and 182, respectively (FIG. 12A). Challenge infection is expected to boost vaccine-induced immune responses and lead to production of antibodies that better neutralize antigens of the respective challenge SARS-CoV-2 variants. Because the inventors collected sera shortly after the challenge, the inventors did not expect to observe the induction of a variant-specific humoral response. Nevertheless, to account for possible challenge-induced effects, the inventors also compared serum neutralization activity as a function of the challenge virus (FIG. 12B). This comparison showed that none of the different challenge viruses had any discernible effect on the neutralizing activity of the hamster sera. In other words, hamsters challenged with one variant, for example, B.1.351, did not produce a better humoral response against the same variant within 3 days after challenge. Furthermore, there was no significant difference in the neutralizing activity of sera collected on day 2 or 3 after challenge.

Vaccination with sCPD9 induced high levels of neutralizing antibodies that were similarly effective against not only the ancestral B.1 variant, which is antigenically identical with the sCPD9 virus, but also the evolutionarily more distant variant B.1.1.7, which has 17 mutations that alter or delete amino acids (8 mutations in the spike protein) compared with the original virus. Hamster sera also effectively neutralized the B.1.351 variant, albeit with six- to ninefold reduced efficacy (FIG. 12C). The results are in good agreement with previous studies, which showed that B.1.351 markedly decreased the neutralization activity of convalescent plasma of individuals that were infected with early SARS-CoV-2 lineages (˜9- to 15-fold reduction) (Cele et al., 2021; Dejnirattisai et al., 2021; Wang et al., 2021) and sera from individuals who have been vaccinated with vaccines containing the original version of spike protein (˜10- to 14-fold reduction) (Wang et al., 2021; Planas et al., 2021a).

Moreover, sera from vaccinated hamsters collected on days 2 and 3 after challenge also efficiently neutralized SARS-CoV-2 variants B.1.128.1 and B.1.617.2 (FIGS. 12A to 12C). These two variants were not used for challenge infection in this study, but were still neutralized with similar efficacy (˜5- to 8-fold reduction in comparison to B.1) as the B.1.351 variant (˜6-fold reduction). This result confirms that vaccination with the vaccine candidate sCPD9 elicits broad and potent neutralizing antibody responses against a wide variety of SARS-CoV-2 variants. The data also show that among the virus variants studied here, variant B.1.617.2 is the most resistant to neutralization with antibodies that were induced by vaccination with a virus that contained the ancestral form of the spike protein. Recent studies have yielded similar results, showing that sera from vaccinated individuals and convalescent sera from individuals previously infected with variants other than B.1.617.2 neutralized the B.1.617.2 variant with a three- to sixfold reduced efficacy (Planas et al., 2021; Liu et al., 2021).

### DISCUSSION

Emerging variants are a constantly growing threat, because they not only dampen the efficacy of the licensed vaccines that target the original SARS-CoV-2 but also raise concerns that the virus may soon become resistant to current vaccines (Moore et al., 2021). Mutations in the spike protein have been shown to be primarily responsible for decreased neutralization of B.1.351 virus because they interfere with the efficient binding of neutralizing antibodies. Of particular importance is the E484K mutation in the receptor-binding domain of the spike protein, which prevents binding of the most potent neutralizing antibodies found in convalescent and post vaccination sera (Yuan et al., 2021). This mutation is also present in B.1.1.28.1 and B.1.525 lineages and was also identified in some viruses of the B.1.1.7 and B.1.526 lineages.

Recent evidence has shown that some of the acquired mutations in the spike protein partially or severely compromise the efficacy of licensed vaccines that are based solely on said spike protein. For example, whereas Pfizer-BioNTech's vaccine (BNT162b2, or Comirnaty) prevents 75% of mild and 90% of severe disease cases caused by the B.1.351 variant (Abu-Raddad et al., 2021), the efficacy of Johnson & Johnson's vaccine (Ad26.COV2.S) is 57 and 100%, respectively (Johnson & Johnson), and the efficacy of Novavax vaccine (NVX-CoV2373) is 49 and 100%, respectively (Shinde et al., 2021). AstraZeneca's vaccine (Vaxzevria or Covishield) has the lowest efficacy of all vaccines licensed in Europe, preventing only 10% infection with the variant B.1.351 (Madhi et al., 2021). Similarly, although inactivated virus vaccines contain the entire antigenic repertoire of the virus and should theoretically provide better efficacy against emerging SARS-CoV-2 variants, in practice they offer the lowest efficacy against early virus variants among all licensed vaccines and therefore can offer only partial protection against emerging variants. For example, in Brazil, where 75% of infections were caused by variant B.1.1.28.1, the efficacy of Sinovac's vaccine (CoronaVac) was only 50% in preventing symptomatic infection (Hitchings et al., 2021).

In the context of continuous SARS-CoV-2 evolution, the study yielded several important findings. The inventors show that single-dose intranasal vaccination with a live attenuated virus vaccine candidate, sCPD9, induced complete protection against clinical disease and lung pathology caused by three different SARS-CoV-2 variants in a highly sensitive animal model. The challenge-infected hamsters did not develop any signs of disease and mounted strong neutralizing antibody responses. The inventors show that B.1.351 virus, although markedly more resistant to neutralization by serum antibodies than B.1 or B.1.1.7 variants, was well controlled by vaccination with sCPD9. The fact that the lungs of B.1.351-challenged hamsters were completely protected from any damage suggests that vaccination with sCPD9 induced effective immune responses and thus compensated the reduced humoral immunity against variant B.1.351. Serum from vaccinated and challenged hamsters also showed neutralizing activity against B.1.1.28.1 and B.1.617.2, with results similar to what was determined for B.1.351. This finding suggests a protective effect of vaccination with sCPD9 against a wide variety of SARS-CoV-2 variants.

Safe blood collection from dwarf hamsters before euthanasia does not yield sufficient serum to determine serum neutralization titers before challenge infection. The lack of access to the sera limits the ability to assess the antibody responses by the vaccine in the absence of infection. Excellent clinical protection, consistent with the absence of replication-competent virus in the lungs of challenged animals, and robust antibody responses shortly after challenge do, however, demonstrate the strong protective potential of this vaccine against different SARS-CoV-2 variants. In addition, vaccination with a live attenuated virus may offer better protection from (re) infection than vaccines that carry only a single antigen, because all SARS-CoV-2 proteins may also trigger stronger cytotoxic T cell responses (Jang et al., 2021).

While other studies have shown that emerging variants threaten the success of vaccination and rekindle the pandemic by diminishing the efficacy of licensed vaccines, the present study shows that a single-dose intranasal vaccination with the lead live attenuated virus candidate sCPD9 induces immunity that provides excellent protection against currently circulating VOCs. It is conceivable that live attenuated virus vaccines, once fully developed, may offer a more practical solution on how to achieve protective immunity against such variants, instead of developing a specialized vaccine against each new dangerous variant that emerges. A live attenuated vaccine as described here may be particularly beneficial to boost and broaden immune responses after primary immunization with other vaccines. It remains to be determined to what extent the vaccination with the sCPD9 virus can protect against emerging VOCs in a nonhuman primate model or in humans.

### Materials and Methods

### Cells and Viruses

Vero E6 cells (American Type Culture Collection, CRL-1586) were grown in minimal essential medium (MEM) containing 10% fetal bovine serum (PAN Biotech), penicillin G (100 IU/ml), and streptomycin (100 μg/ml; Carl Roth) at 37° C. and 5% CO₂. SARS-CoV-2 variants B.1 (BetaCoV/Munich/BavPat1/2020) (Wölfel et al., 2020), B.1.1.7 (BetaCoV/Germany/ChVir21652/2020), B.1.351 (hCoV-19/Netherlands/NoordHolland_20159/2021), B.1.1.28.1 (hCoV-19/Netherlands/NoordHolland_10915/2021), and B.1.617.2 [SARS-CoV-2, Human, 2021, Germany ex India, 20A/452R (B.1.617)] were grown in Vero E6 cells. Virus stocks were stored at −80° C. before experimental infections.

Production of High-Titer Virus Stocks of sCPD9

To produce high-titer virus stocks, confluent monolayers of Vero E6 cells grown in T-75 flasks were infected with sCPD9 virus at a multiplicity of infection of 1. Infected cells were cultured in 7 ml of cell culture medium and harvested at the onset of cytopathic effect on days 2 to 3 after infection. The virus stocks prepared in this manner typically contain approximately 1×10⁷ infectious virus particles per milliliter of cell culture.

### Virus Titrations

The concentration of infectious viruses in virus stocks and in the lungs of infected animals was determined by titration on Vero E6 cells grown in 12-well plates. Stock titers of WT SARS-CoV-2 variants were determined by a plaque assay. Because sCPD9 virus does not produce readily visible plaques on Vero E6 cells, the titers of sCPD9 virus stocks were determined by a focus-forming assay. Briefly, Vero E6 cells were infected with 100 μl of serial 10-fold dilutions of virus. After 2 hours of incubation, the viral inoculum was removed and cells were overlaid with Dulbecco's modified Eagle's medium (DMEM) containing 2.5% microcrystalline cellulose (Avicel, FMC BioPolymer). Seventy-two hours after infection, cells were fixed with 4% formaldehyde, permeabilized with 0.1% Triton X-100, and blocked with 3% bovine serum albumin in phosphate-buffered saline (PBS). Cells were then incubated with a monoclonal mouse anti-SARS-CoV-2 nucleocapsid antibody (provided by S. Reiche, Friedrich Loeffler Institute, Riems, Germany) for 1 hour and then with goat anti-mouse immunoglobulin G-Alexa Fluor 568 secondary antibody (Thermo Fisher Scientific) for 45 min. To determine virus titers in the lung, 50 mg of lung tissue was first homogenized with a bead mill (Analytic Jena), the homogenate was serially diluted, and the virus titer was determined by focus-forming assay as described above.

After challenge infection of hamsters, virus titers in the lung of challenged animals were determined by plating 10-fold serial dilutions of 50 mg of lung homogenates onto Vero E6 cells. After incubation at 37° C. for 2 hours, the homogenized tissue was aspirated, and cells were washed with PBS and overlaid with 2.5% microcrystalline cellulose (Avicel, FMC BioPolymer). Seventy-two hours after infection, cells were fixed with 4% formaldehyde and stained with crystal violet (0.75% aqueous solution), and plaques were counted.

### Ethics Statement

In vitro and animal work was done under biosafety conditions in the biosafety level-3 (BSL-3) facility at the Institut für Virologie, Freie Universität Berlin, Germany. All animal experiments were approved by the Landesamt für Gesundheit und Soziales in Berlin, Germany (permit number 0086/20) and performed in compliance with relevant institutional, national, and international guidelines for care and humane use of animal subjects.

### Animal Husbandry

The animal experiments were done in a certified BSL-3 facility. Five- to seven-week-old Roborovski dwarf hamsters (P. roborovskii) were purchased through the German pet trade from a single breeding facility. They were housed in groups of six animals in individually ventilated cages. The animals had unrestricted access to food and water and were allowed to acclimate to the conditions for 7 days before infection. During both experiments, cage temperatures ranged from 22° to 24° C. and relative humidity ranged from 40 to 55%.

### Vaccination and Challenge Experiments

To assess the vaccine efficacy of the lead attenuated virus vaccine candidate sCPD9 in the Roborovski dwarf hamster, animals were randomly assigned into two groups, with 60% of the animals in each group being female. Twelve hamsters were mock-vaccinated with cell culture medium, and 30 hamsters were vaccinated with 1×10⁵ FFU of the vaccine candidate sCPD9 virus in 20 μl by intranasal instillation under anesthesia (Osterrieder et al., 2020). Twenty-one days after vaccination, hamsters were challenge-infected with 1×10⁵ PFUs of challenge virus in 20 μl by intranasal instillation. Nine mock-vaccinated hamsters were challenged with the SARS-CoV-2 variant B.1, and nine sCPD9-vaccinated hamsters were challenged with either the SARS-CoV-2 variant B.1, B.1.1.7, or B.1.351. During the experiment, all hamsters were monitored twice daily for the clinical signs of disease and body weights were recorded. On day 3 after vaccination, and on days 2, 3, and 5 days after challenge (days 23, 24, and 26 of the experiment), three hamsters of each group were euthanized. Blood, tracheal swabs, and lungs were collected to determine virological and histological parameters of vaccination or infection. The left lungs were preserved in 4% formalin for subsequent histopathological detailed investigations.

### Histopathology and In Situ Hybridization

For histopathology, the left lung lobe was carefully removed and immersion-fixed in buffered 4% formalin, pH 7.0, for 48 hours. Lungs were embedded in paraffin and cut at 2 μm thickness. Sections were stained with hematoxylin and eosin (H & E) and periodic acid-Schiff (PAS) reaction followed by blinded microscopic evaluation by board-certified veterinary pathologist (A. D. G.) (Gruber et al., 2020). H & E- and PAS-stained and in situ hybridization slides were analyzed and photographed using an Olympus BX41 microscope with the DP80 Microscope Digital Camera and the cellSens Imaging Software, version 1.18 (Olympus Corporation). For overviews with lower magnification, slides were automatically digitized using an Aperio CS2 slide scanner (Leica Biosystems), and image files were generated using the Image Scope Software (Leica Biosystems).

### RNA Isolation and Reverse Transcription Quantitative Polymerase Chain Reaction

RNA was extracted from 25 mg of lung homogenates and tracheal swabs using the innuPREP Virus RNA Kit (Analytik Jena). SARS-CoV-2 RNA copies were quantified using the NEB Luna Universal Probe One-Step RT-qPCR Kit (New England Biolabs) on the StepOnePlus Real-Time PCR System (Thermo Fisher Scientific) as previously described (Corman et al., 2020).

### Serum Virus Neutralization Assay

Microneutralization assay was done with six sera from hamsters that were mock-vaccinated and challenge-infected with the B.1 virus variant, and with five, six, and six sera from hamsters that were sCPD9-vaccinated and challenge-infected with the B.1, B.1.1.7, or B.1.351 virus variants, respectively. Each serum was tested for neutralization of the B.1, B.1.1.7, B.1.351, B.1.1.28.1, or B.1.617.2 variant. Microneutralization assay was performed by twofold serial dilutions (1:16 to 1:2048) of complement-inactivated (56° C., 30 min) hamster serum plated on subconfluent monolayers of Vero E6 cells in 96-well plates. Cells in each well were infected with 100 PFUs of the respective SARS-CoV-2 variant, incubated for 72 hours at 37° C., fixed with 4% formalin for 24 hours, and stained with crystal violet (0.75% aqueous solution). Serum neutralization was considered effective in wells that showed no any cytopathic effect, and the last effective dilution was counted.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this solution.

### LIST OF REFERENCES CITED IN THE PRECEDING SECTIONS

1. Johnson & Johnson COVID-19 Vaccine Authorized by U. S. FDA For Emergency Use—First Single-Shot Vaccine in Fight Against Global Pandemic https://www.jnj.com/johnson-johnson-covid-19-vaccine-authorized-by-u-s-fda-for-emergency-usefirst-single-shot-vaccine-in-fight-against-global-pandemic #_edn2 (Johnson & Johnson).
2. The World Health Organization. Draft landscape of COVID-19 candidate vaccines. https://www.who.int/publications/m/item/draft-landscape-of-covid-19-candidate-vaccines (WHO).
3. Abu-Raddad, L. J., Chemaitelly, H., Butt, A. A., and National Study Group for, C. V. (2021). Effectiveness of the BNT162b2 Covid-19 Vaccine against the B.1.1.7 and B.1.351 Variants. N Engl J Med.
4. Almazan, F., Gonzalez, J. M., Penzes, Z., Izeta, A., Calvo, E., Plana-Duran, J., and Enjuanes, L. (2000). Engineering the largest RNA virus genome as an infectious bacterial artificial chromosome. Proceedings of the National Academy of Sciences of the United States of America 97, 5516-5521.
5. Anderson, E. J., Rouphael, N. G., Widge, A. T., Jackson, L. A., Roberts, P. C., Makhene, M., Chappell, J. D., Denison, M. R., Stevens, L. J., Pruijssers, A. J., et al. (2020). Safety and Immunogenicity of SARS-CoV-2 mRNA-1273 Vaccine in Older Adults. N Engl J Med 383, 2427-2438.
6. Baden, L. R., El Sahly, H. M., Essink, B., Kotloff, K., Frey, S., Novak, R., Diemert, D., Spector, S. A., Rouphael, N., Creech, C. B., et al. (2021). Efficacy and Safety of the mRNA-1273 SARS-CoV-2 Vaccine. N Engl J Med 384, 403-416.
7. Bazin, H. (2003). A brief history of the prevention of infectious diseases by immunisations. Comparative immunology, microbiology and infectious diseases 26, 293-308.
8 Broadbent, A. J., Santos, C. P., Anafu, A., Wimmer, E., Mueller, S., and Subbarao, K. (2016). Evaluation of the attenuation, immunogenicity, and efficacy of a live virus vaccine generated by codon-pair bias de-optimization of the 2009 pandemic H1N1 influenza virus, in ferrets. Vaccine 34, 563-570.
9. S. Cele, I. Gazy, L. Jackson, S. H. Hwa, H. Tegally, G. Lustig, J. Giandhari, S. Pillay, E. Wilkinson, Y. Naidoo, et al., A. Network for Genomic Surveillance in South, C. K. Team, R. J. Lessells, T. de Oliveira, A. Sigal, Escape of SARS-CoV-2 501Y. V2 from neutralization by convalescent plasma. Nature 593, 142-146 (2021).
10. Chan, J. F., Yuan, S., Kok, K. H., To, K. K., Chu, H., Yang, J., Xing, F., Liu, J., Yip, C. C., Poon, R. W., et al. (2020). A familial cluster of pneumonia associated with the 2019 novel coronavirus indicating person-to-person transmission: a study of a family cluster. Lancet 395, 514-523.
11. Chen, N., Zhou, M., Dong, X., Qu, J., Gong, F., Han, Y., Qiu, Y., Wang, J., Liu, Y., Wei, Y., et al. (2020). Epidemiological and clinical characteristics of 99 cases of 2019 novel coronavirus pneumonia in Wuhan, China: a descriptive study. Lancet 395, 507-513.
12. Coleman, J. R., Papamichail, D., Skiena, S., Futcher, B., Wimmer, E., and Mueller, S. (2008). Virus attenuation by genome-scale changes in codon pair bias. Science 320, 1784-1787.
13. Corbett, K. S., Flynn, B., Foulds, K. E., Francica, J. R., Boyoglu-Barnum, S., Werner, A. P., Flach, B., O'Connell, S., Bock, K. W., Minai, M., et al. (2020). Evaluation of the mRNA-1273 Vaccine against SARS-CoV-2 in Nonhuman Primates. N Engl J Med 383, 1544-1555.
14. Corman, V. M., Landt, O., Kaiser, M., Molenkamp, R., Meijer, A., Chu, D. K., Bleicker, T., Brunink, S., Schneider, J., Schmidt, M. L., et al. (2020). Detection of 2019 novel coronavirus (2019-nCOV) by real-time RT-PCR. Euro Surveill 25.
15. Dagan, N., Barda, N., Kepten, E., Miron, O., Perchik, S., Katz, M. A., Hernan, M. A., Lipsitch, M., Reis, B., and Balicer, R. D. (2021). BNT162b2 mRNA Covid-19 Vaccine in a Nationwide Mass Vaccination Setting. N Engl J Med 384, 1412-1423.
16. N. G. Davies, S. Abbott, R. C. Barnard, C. I. Jarvis, A. J. Kucharski, J. D. Munday, C. A. B. Pearson, T. W. Russell, D. C. Tully, A. D. Washburne, T. Wenseleers, A. Gimma, W. Waites, K. L. M. Wong, K. van Zandvoort, J. D. Silverman, C. C. W. Group, C. G. U. Consortium, K. Diaz-Ordaz, R. Keogh, R. M. Eggo, S. Funk, M. Jit, K. E. Atkins, W. J. Edmunds, Estimated transmissibility and impact of SARS-CoV-2 lineage B.1.1.7 in England. Science 372, (2021).
17. W. Dejnirattisai, D. Zhou, P. Supasa, C. Liu, A. J. Mentzer, H. M. Ginn, Y. Zhao, H. M. E. Duyvesteyn, A. Tuekprakhon, R. Nutalai, et al., Antibody evasion by the P.1 strain of SARS-CoV-2. Cell 184, 2939-2954 e2939 (2021).
18. Dong, E., Du, H., and Gardner, L. (2020). An interactive web-based dashboard to track COVID-19 in real time. Lancet Infect Dis 20, 533-534.
19. O. Dyer, Covid-19: Delta infections threaten herd immunity vaccine strategy. BMJ 374, n1933 (2021).
20. Ella, R., Reddy, S., Jogdand, H., Sarangi, V., Ganneru, B., Prasad, S., Das, D., Raju, D., Praturi, U., Sapkal, G., et al. (2021). Safety and immunogenicity of an inactivated SARS-CoV-2 vaccine, BBV152: interim results from a double-blind, randomised, multicentre, phase 2 trial, and 3-month follow-up of a double-blind, randomised phase 1 trial. Lancet Infect Dis.
21. Emary, K. R. W., Golubchik, T., Aley, P. K., Ariani, C. V., Angus, B., Bibi, S., Blane, B., Bonsall, D., Cicconi, P., Charlton, S., et al. (2021). Efficacy of ChAdOx1 nCoV-19 (AZD1222) vaccine against SARS-CoV-2 variant of concern 202012/01 (B.1.1.7): an exploratory analysis of a randomised controlled trial. Lancet 397, 1351-1362.
22. Eschke, K., Trimpert, J., Osterrieder, N., and Kunec, D. (2018). Attenuation of a very virulent Marek's disease herpesvirus (MDV) by codon pair bias deoptimization. PLoS Pathog 14, e1006857.
23. N. R. Faria, T. A. Mellan, C. Whittaker, I. M. Claro, D. D. S. Candido, S. Mishra, M. A. E. Crispim, F. C. S. Sales, I. Hawryluk, J. T. McCrone et al., Genomics and epidemiology of the P.1 SARS-CoV-2 lineage in Manaus, Brazil. Science 372, 815-821 (2021).
24. Gao, Q., Bao, L., Mao, H., Wang, L., Xu, K., Yang, M., Li, Y., Zhu, L., Wang, N., Lv, Z., et al. (2020). Development of an inactivated vaccine candidate for SARS-CoV-2. Science 369, 77-81.
25. Garg, S., Kim, L., Whitaker, M., O'Halloran, A., Cummings, C., Holstein, R., Prill, M., Chai, S. J., Kirley, P. D., Alden, N. B., et al. (2020). Hospitalization Rates and Characteristics of Patients Hospitalized with Laboratory-Confirmed Coronavirus Disease 2019-COVID-NET, 14 States, Mar. 1-30, 2020. MMWR Morb Mortal Wkly Rep 69, 458-464.
26. Greinacher, A., Thiele, T., Warkentin, T. E., Weisser, K., Kyrle, P. A., and Eichinger, S. (2021). Thrombotic Thrombocytopenia after ChAdOx1 nCov-19 Vaccination. N Engl J Med.
27. Groenke, N., Trimpert, J., Merz, S., Conradie, A. M., Wyler, E., Zhang, H., Hazapis, O. G., Rausch, S., Landthaler, M., Osterrieder, N., et al. (2020). Mechanism of Virus Attenuation by Codon Pair Deoptimization. Cell Rep 31, 107586.
28. Gruber, A. D., Osterrieder, N., Bertzbach, L. D., Vladimirova, D., Greuel, S., Ihlow, J., Horst, D., Trimpert, J., and Dietert, K. (2020). Standardization of Reporting Criteria for Lung Pathology in SARS-CoV-2 Infected Hamsters-What Matters? Am J Respir Cell Mol Biol 63, 856-859.
29. G. A. Gutman, G. W. Hatfield, Nonrandom utilization of codon pairs in Escherichia coli. Proc Natl Acad Sci USA 86, 3699-3703 (1989).
30. Haas, E. J., Angulo, F. J., Mclaughlin, J. M., Anis, E., Singer, S. R., Khan, F., Brooks, N., Smaja, M., Mircus, G., Pan, K., et al. (2021). Impact and effectiveness of mRNA BNT162b2 vaccine against SARS-CoV-2 infections and COVID-19 cases, hospitalisations, and deaths following a nationwide vaccination campaign in Israel: an observational study using national surveillance data. Lancet.
31. Hall, V. J., Foulkes, S., Saei, A., Andrews, N., Oguti, B., Charlett, A., Wellington, E., Stowe, J., Gillson, N., Atti, A., et al. (2021). COVID-19 vaccine coverage in health-care workers in England and effectiveness of BNT162b2 mRNA vaccine against infection (SIREN): a prospective, multicentre, cohort study. Lancet.
32. M. D. T. Hitchings, O. T. Ranzani, M. S. Scaramuzzini Torres, S. B. de Oliveira, M. Almiron, R. Said, R. Borg, W. L. Schulz, R. D. de Oliveira, P. V. da Silva, et al., Effectiveness of CoronaVac in the setting of high SARS-CoV-2 P.1 variant transmission in Brazil: A test-negative case-control study. medRxiv, 2021.2004.2007.21255081 (2021).
33. Y. H. Jang, B. L. Seong, Immune Responses Elicited by Live Attenuated Influenza Vaccines as Correlates of Universal Protection against Influenza Viruses. Vaccines (Basel) 9, (2021).
34. Jackson, L. A., Anderson, E. J., Rouphael, N. G., Roberts, P. C., Makhene, M., Coler, R. N., McCullough, M. P., Chappell, J. D., Denison, M. R., Stevens, L. J., et al. (2020). An mRNA Vaccine against SARS-CoV-2-Preliminary Report. N Engl J Med.
35. Kew, O. M., Sutter, R. W., de Gourville, E. M., Dowdle, W. R., and Pallansch, M. A. (2005). Vaccine-derived polioviruses and the endgame strategy for global polio eradication. Annu Rev Microbiol 59, 587-635.
36. Khedkar, P. H., Osterrieder, N., and Kunec, D. (2018). Codon pair bias deoptimization of the major oncogene meq of a very virulent Marek's disease virus. J Gen Virol 99, 1705-1716.
37. Kunec, D., and Osterrieder, N. (2016). Codon Pair Bias Is a Direct Consequence of Dinucleotide Bias. Cell Rep 14, 55-67.
38. Kusters, I., and Almond, J. W. (2008). Vaccine Strategies. In Encyclopedia of Virology (Third Edition), B. W. J. M. Editors-in-Chief: and M. H. V. v. Regenmortel, eds. (Oxford: Academic Press), pp. 235-243.
39. Kutter, J. S., de Meulder, D., Bestebroer, T. M., Lexmond, P., Mulders, A., Richard, M., Fouchier, R. A. M., and Herfst, S. (2021). SARS-COV and SARS-CoV-2 are transmitted through the air between ferrets over more than one meter distance. Nat Commun 12, 1653.
40. Lauring, A. S., Acevedo, A., Cooper, S. B., and Andino, R. (2012). Codon usage determines the mutational robustness, evolutionary capacity, and virulence of an RNA virus. Cell Host Microbe 12, 623-632.
41. Lauring, A. S., Jones, J. O., and Andino, R. (2010). Rationalizing the development of live attenuated virus vaccines. Nature biotechnology 28, 573-579.
42. Le Nouen, C., Brock, L. G., Luongo, C., McCarty, T., Yang, L., Mehedi, M., Wimmer, E., Mueller, S., Collins, P. L., Buchholz, U. J., et al. (2014). Attenuation of human respiratory syncytial virus by genome-scale codon-pair deoptimization. Proceedings of the National Academy of Sciences of the United States of America 111, 13169-13174.
43. C. Liu, H. M. Ginn, W. Dejnirattisai, P. Supasa, B. Wang, A. Tuekprakhon, R. Nutalai, D. Zhou, A. J. Mentzer, Y. Zhao, et al., Reduced neutralization of SARS-CoV-2 B.1.617 by vaccine and convalescent serum. Cell, (2021).
44. Logunov, D. Y., Dolzhikova, I. V., Shcheblyakov, D. V., Tukhvatulin, A. I., Zubkova, O. V., Dzharullaeva, A. S., Kovyrshina, A. V., Lubenets, N. L., Grousova, D. M., Erokhova, A. S., et al. (2021). Safety and efficacy of an rAd26 and rAd5 vector-based heterologous prime-boost COVID-19 vaccine: an interim analysis of a randomised controlled phase 3 trial in Russia. Lancet 397, 671-681.
45. Madhi, S. A., Baillie, V., Cutland, C. L., Voysey, M., Koen, A. L., Fairlie, L., Padayachee, S. D., Dheda, K., Barnabas, S. L., Bhorat, Q. E., et al. (2021). Efficacy of the ChAdOx1 nCOV-19 Covid-19 Vaccine against the B.1.351 Variant. N Engl J Med.
46. Mallapaty, S. (2021). China's COVID vaccines are going global—but questions remain. Nature.
47. J. P. Moore, P. A. Offit, SARS-CoV-2 Vaccines and the Growing Threat of Viral Variants. JAMA 325, 821-822 (2021).
48. Mueller, S., Coleman, J. R., Papamichail, D., Ward, C. B., Nimnual, A., Futcher, B., Skiena, S., and Wimmer, E. (2010). Live attenuated influenza virus vaccines by computer-aided rational design. Nature biotechnology 28, 723-726.
49. Mulligan, M. J., Lyke, K. E., Kitchin, N., Absalon, J., Gurtman, A., Lockhart, S., Neuzil, K., Raabe, V., Bailey, R., Swanson, K. A., et al. (2020). Phase I/II study of COVID-19 RNA vaccine BNT162b1 in adults. Nature.
50. Nakamura, T., Karakida, N., Dantsuka, A., Ichii, O., Elewa, Y. H. A., Kon, Y., Nagasaki, K. I., Hattori, H., and Yoshiyasu, T. (2017). Effects of a mixture of medetomidine, midazolam and butorphanol on anesthesia and blood biochemistry and the antagonizing action of atipamezole in hamsters. J Vet Med Sci 79, 1230-1235.
51. F. G. Naveca, V. Nascimento, V. C. de Souza, A. L. Corado, F. Nascimento, G. Silva, A. Costa, D. Duarte, K. Pessoa, M. Mejia, M. J. Brandao, M. Jesus, L. Goncalves, C. F. da Costa, V. Sampaio, D. Barros, M. Silva, T. Mattos, G. Pontes, L. Abdalla, J. H. Santos, I. Arantes, F. Z. Dezordi, M. M. Siqueira, G. L. Wallau, P. C. Resende, E. Delatorre, T. Graf, G. Bello, COVID-19 in Amazonas, Brazil, was driven by the persistence of endemic lineages and P.1 emergence. Nat Med, (2021).
52. Noskov, V., Kouprina, N., Leem, S. H., Koriabine, M., Barrett, J. C., and Larionov, V. (2002). A genetic system for direct selection of gene-positive clones during recombinational cloning in yeast. Nucleic Acids Res 30, E8.
53. Odon, V., Fros, J. J., Goonawardane, N., Dietrich, I., Ibrahim, A., Alshaikhahmed, K., Nguyen, D., and Simmonds, P. (2019). The role of ZAP and OAS3/RNAseL pathways in the attenuation of an RNA virus with elevated frequencies of CpG and UpA dinucleotides. Nucleic Acids Res 47, 8061-8083.
54. Osterrieder, N., Bertzbach, L. D., Dietert, K., Abdelgawad, A., Vladimirova, D., Kunec, D., Hoffmann, D., Beer, M., Gruber, A. D., and Trimpert, J. (2020). Age-Dependent Progression of SARS-CoV-2 Infection in Syrian Hamsters. Viruses 12.
55. Osterrieder, N., and Kunec, D. (2018). Attenuation of Viruses by Large-Scale Recoding of their Genomes: the Selection Is Always Biased. Curr Clin Microbiol Rep 5, 66-72.
56. D. Planas, T. Bruel, L. Grzelak, F. Guivel-Benhassine, I. Staropoli, F. Porrot, C. Planchais, J. Buchrieser, M. M. Rajah, E. Bishop, et al., Sensitivity of infectious SARS-CoV-2 B.1.1.7 and B.1.351 variants to neutralizing antibodies. Nat Med 27, 917-924 (2021a).
57. D. Planas, D. Veyer, A. Baidaliuk, I. Staropoli, F. Guivel-Benhassine, M. M. Rajah, C. Planchais, F. Porrot, N. Robillard, J. Puech, et al., Reduced sensitivity of SARS-CoV-2 variant Delta to antibody neutralization. Nature, (2021b).
58. Polack, F. P., Thomas, S. J., Kitchin, N., Absalon, J., Gurtman, A., Lockhart, S., Perez, J. L., Perez Marc, G., Moreira, E. D., Zerbini, C., et al. (2020). Safety and Efficacy of the BNT162b2 mRNA Covid-19 Vaccine. N Engl J Med.
59. Sahin, U., Muik, A., Derhovanessian, E., Vogler, I., Kranz, L. M., Vormehr, M., Baum, A., Pascal, K., Quandt, J., Maurus, D., et al. (2020). COVID-19 vaccine BNT162b1 elicits human antibody and THI T-cell responses. Nature.
60. Schneider, C. A., Rasband, W. S., and Eliceiri, K. W. (2012). NIH Image to ImageJ: 25 years of image analysis. Nat Methods 9, 671-675.
61. Shen, S. H., Stauft, C. B., Gorbatsevych, O., Song, Y., Ward, C. B., Yurovsky, A., Mueller, S., Futcher, B., and Wimmer, E. (2015). Large-scale recoding of an arbovirus genome to rebalance its insect versus mammalian preference. Proceedings of the National Academy of Sciences of the United States of America.
62. V. Shinde, S. Bhikha, Z. Hoosain, M. Archary, Q. Bhorat, L. Fairlie, U. Lalloo, M. S. L. Masilela, D. Moodley, S. Hanley, et al., Efficacy of NVX-CoV2373 Covid-19 Vaccine against the B.1.351 Variant. N Engl J Med 384, 1899-1909 (2021).
63. Sia, S. F., Yan, L. M., Chin, A. W. H., Fung, K., Poon, L. L. M., Nicholls, J. M., Peiris, M., and Yen, H. L. (2020). Pathogenesis and transmission of SARS-CoV-2 virus in golden Syrian hamsters.
64. Solforosi, L., Kuipers, H., Jongeneelen, M., Rosendahl Huber, S. K., van der Lubbe, J. E. M., Dekking, L., Czapska-Casey, D. N., Izquierdo Gil, A., Baert, M. R. M., Drijver, J., et al. (2021). Immunogenicity and efficacy of one and two doses of Ad26.COV2.S COVID vaccine in adult and aged NHP. J Exp Med 218.
65. Song, Y., Liu, Y., Ward, C. B., Mueller, S., Futcher, B., Skiena, S., Paul, A. V., and Wimmer, E. (2012). Identification of two functionally redundant RNA elements in the coding sequence of poliovirus using computer-generated design. Proceedings of the National Academy of Sciences of the United States of America 109, 14301-14307.
66. H. Tegally, E. Wilkinson, R. J. Lessells, J. Giandhari, S. Pillay, N. Msomi, K. Mlisana, J. N. Bhiman, A. von Gottberg, S. Walaza, V. Fonseca, M. Allam, A. Ismail, A. J. Glass, S. Engelbrecht, G. Van Zyl, W. Preiser, C. Williamson, F. Petruccione, A. Sigal, I. Gazy, D. Hardie, N. Y. Hsiao, D. Martin, D. York, D. Goedhals, E. J. San, M. Giovanetti, J. Lourenco, L. C. J. Alcantara, T. de Oliveira, Sixteen novel lineages of SARS-CoV-2 in South Africa. Nat Med 27, 440-446 (2021).
67. Thi Nhu Thao, T., Labroussaa, F., Ebert, N., V'Kovski, P., Stalder, H., Portmann, J., Kelly, J., Steiner, S., Holwerda, M., Kratzel, A., et al. (2020). Rapid reconstruction of SARS-CoV-2 using a synthetic genomics platform. Nature 582, 561-565.
68. Trimpert, J., Adler, J. M., Eschke, K., Abdelgawad, A., Firsching, T. C., Ebert, N., Thao, T. T. N., Gruber, A. D., Thiel, V., Osterrieder, N., et al. (2021a). Live attenuated virus vaccine protects against SARS-CoV-2 variants of concern B.1.1.7 (Alpha) and B.1.351 (Beta). Sci Adv 7, eabk0172.
69. Trimpert, J., Dietert, K., Firsching, T. C., Ebert, N., Thi Nhu Thao, T., Vladimirova, D., Kaufer, S., Labroussaa, F., Abdelgawad, A., Conradie, A., et al. (2021b). Development of safe and highly protective live-attenuated SARS-CoV-2 vaccine candidates by genome recoding. Cell Rep 36, 109493.
70. Trimpert, T., Vladimirova, D., Dietert, K., Abdelgawad, A., Kunec, D., Dökel, D., Gruber, A. D., Bertzbach, L., and Osterrieder, N. (2020). The Roborovski dwarf hamster—a highly susceptible model for a rapid and fatal course of SARS-CoV-2 infection. Cell Reports 33, 108488.
71. E. Volz, S. Mishra, M. Chand, J. C. Barrett, R. Johnson, L. Geidelberg, W. R. Hinsley, D. J. Laydon, G. Dabrera, A. O'Toole, R. Amato, M. Ragonnet-Cronin, I. Harrison, B. Jackson, C. V. Ariani, O. Boyd, N. J. Loman, J. T. McCrone, S. Goncalves, D. Jorgensen, R. Myers, V. Hill, D. K. Jackson, K. Gaythorpe, N. Groves, J. Sillitoe, D. P. Kwiatkowski, C. G. U. consortium, S. Flaxman, O. Ratmann, S. Bhatt, S. Hopkins, A. Gandy, A. Rambaut, N. M. Ferguson, Assessing transmissibility of SARS-CoV-2 lineage B.1.1.7 in England. Nature 593, 266-269 (2021).
72. Voysey, M., Clemens, S. A. C., Madhi, S. A., Weckx, L. Y., Folegatti, P. M., Aley, P. K., Angus, B., Baillie, V. L., Barnabas, S. L., Bhorat, Q. E., et al. (2021a). Safety and efficacy of the ChAdOx1 nCOV-19 vaccine (AZD1222) against SARS-CoV-2: an interim analysis of four randomised controlled trials in Brazil, South Africa, and the UK. Lancet 397, 99-111.
73. Voysey, M., Costa Clemens, S. A., Madhi, S. A., Weckx, L. Y., Folegatti, P. M., Aley, P. K., Angus, B., Baillie, V. L., Barnabas, S. L., Bhorat, Q. E., et al. (2021b). Single-dose administration and the influence of the timing of the booster dose on immunogenicity and efficacy of ChAdOx1 nCOV-19 (AZD1222) vaccine: a pooled analysis of four randomised trials. Lancet 397, 881-891.
74. Walsh, E. E., Frenck, R. W., Jr., Falsey, A. R., Kitchin, N., Absalon, J., Gurtman, A., Lockhart, S., Neuzil, K., Mulligan, M. J., Bailey, R., et al. (2020). Safety and Immunogenicity of Two RNA-Based Covid-19 Vaccine Candidates. N Engl J Med 383, 2439-2450.
75. Wang, H., Zhang, Y., Huang, B., Deng, W., Quan, Y., Wang, W., Xu, W., Zhao, Y., Li, N., Zhang, J., et al. (2020). Development of an Inactivated Vaccine Candidate, BBIBP-CorV, with Potent Protection against SARS-CoV-2. Cell 182, 713-721 e719.
76. P. Wang, M. S. Nair, L. Liu, S. Iketani, Y. Luo, Y. Guo, M. Wang, J. Yu, B. Zhang, P. D. Kwong, B. S. Graham, J. R. Mascola, J. Y. Chang, M. T. Yin, M. Sobieszczyk, C. A. Kyratsous, L. Shapiro, Z. Sheng, Y. Huang, D. D. Ho, Antibody resistance of SARS-CoV-2 variants B.1.351 and B.1.1.7. Nature 593, 130-135 (2021).
77. Wimmer, E., Mueller, S., Tumpey, T. M., and Taubenberger, J. K. (2009). Synthetic viruses: a new opportunity to understand and prevent viral disease. Nature biotechnology 27, 1163-1172.
78. Wölfel, R., Corman, V. M., Guggemos, W., Seilmaier, M., Zange, S., Müller, M. A., Niemeyer, D., Jones, T. C., Vollmar, P., Rothe, C., et al. (2020). Virological assessment of hospitalized patients with COVID-2019. Nature 581, 465-469.
79. Wu, A., Peng, Y., Huang, B., Ding, X., Wang, X., Niu, P., Meng, J., Zhu, Z., Zhang, Z., Wang, J., et al. (2020). Genome Composition and Divergence of the Novel Coronavirus (2019-nCOV) Originating in China. Cell Host Microbe 27, 325-328.
80. M. Yuan, D. Huang, C. D. Lee, N. C. Wu, A. M. Jackson, X. Zhu, H. Liu, L. Peng, M. J. van Gils, R. W. Sanders, et al., Structural and functional ramifications of antigenic drift in recent SARS-CoV-2 variants. Science, (2021).
81. Zhang, Y., Zeng, G., Pan, H., Li, C., Hu, Y., Chu, K., Han, W., Chen, Z., Tang, R., Yin, W., et al. (2021). Safety, tolerability, and immunogenicity of an inactivated SARS-CoV-2 vaccine in healthy adults aged 18-59 years: a randomised, double-blind, placebo-controlled, phase 1/2 clinical trial. Lancet Infect Dis 21, 181-192.
82. Zhou, F., Yu, T., Du, R., Fan, G., Liu, Y., Liu, Z., Xiang, J., Wang, Y., Song, B., Gu, X., et al. (2020a). Clinical course and risk factors for mortality of adult inpatients with COVID-19 in Wuhan, China: a retrospective cohort study. Lancet 395, 1054-1062.
83. Zhou, P., Yang, X. L., Wang, X. G., Hu, B., Zhang, L., Zhang, W., Si, H. R., Zhu, Y., Li, B., Huang, C. L., et al. (2020b). A pneumonia outbreak associated with a new coronavirus of probable bat origin. Nature 579, 270-273.
84. Zhu, F. C., Guan, X. H., Li, Y. H., Huang, J. Y., Jiang, T., Hou, L. H., Li, J. X., Yang, B. F., Wang, L., Wang, W. J., et al. (2020). Immunogenicity and safety of a recombinant adenovirus type-5-vectored COVID-19 vaccine in healthy adults aged 18 years or older: a randomised, double-blind, placebo-controlled, phase 2 trial. Lancet 396, 479-488.
85. Zimmer, C., Corum, J., and Wee, S. L. (2021). Coronavirus Vaccine Tracker. The New York Times.
86. Zou, L., Ruan, F., Huang, M., Liang, L., Huang, H., Hong, Z., Yu, J., Kang, M., Song, Y., Xia, J., et al. (2020). SARS-CoV-2 Viral Load in Upper Respiratory Specimens of Infected Patients. N Engl J Med 382, 1177-1179.

1. A polynucleotide encoding
a) severe acute respiratory syndrome coronavirus 2 (SARS-CoV-2) spike protein; and/or
b) at least one non-structural SARS-CoV-2 protein selected from the group consisting of non-structural protein 7, non-structural protein 8, non-structural protein 9, non-structural protein 10, non-structural protein 11, non-structural protein 12, an endoribonuclease, and a 2′-O-methyltransferase,
wherein the polynucleotide comprises or consists of at least one sequence part comprising codon-pair deoptimizations in comparison to the SARS-CoV-2 genome.

2. The polynucleotide of claim 1, wherein the polynucleotide encodes at least two of the non-structural proteins.

3. The polynucleotide of claim 1, wherein the SARS-CoV-2 genome is the SARS-CoV-2 genome section extending from position 11,000 to position 27,000.

4. The polynucleotide of claim 1, wherein the at least one sequence part comprising codon-pair deoptimizations has a length in a range of from 750 nucleotides to 2500 nucleotides.

5. The polynucleotide of claim 1, wherein between 15% and 40% of the nucleotides of the at least one sequence part comprising codon-pair deoptimizations are different from the nucleotides of a corresponding SARS-CoV-2 genome.

6. The polynucleotide of claim 1, wherein the at least one sequence part comprising codon-pair deoptimizations comprises between 200 and 500 nucleotides that are different from the nucleotides of a corresponding SARS-CoV-2 genome.

7. The polynucleotide of claim 1, wherein between 40% and 70% of the codons of the at least one sequence part comprising codon-pair deoptimizations are different from the codons of a corresponding SARS-CoV-2 genome.

8. The polynucleotide of claim 1, wherein the at least one sequence part comprising codon-pair deoptimizations comprises between 150 and 400 codons that are different from the codons of a corresponding SARS-CoV-2 genome.

9. The polynucleotide of claim 1, wherein the at least one sequence part comprising codon-pair deoptimizations comprises a first deoptimized sequence part and a second deoptimized sequence part, wherein the first deoptimized sequence part and the second deoptimized sequence part are separated from each other by a non-deoptimized sequence section comprising at least 300 nucleotides.

10. The polynucleotide of claim 9, wherein the first deoptimized sequence part has a length lying in a range of from 1300 nucleotides to 1600 nucleotides and the second deoptimized sequence part has a length lying in a range of from 100 nucleotides to 400 nucleotides.

11. The polynucleotide of claim 9, wherein the first deoptimized sequence part has at least 95% sequence identity to SEQ ID NO. 2 and the second deoptimized sequence part has at least 95% sequence identity to SEQ ID NO. 4.

12. The polynucleotide of claim 1, wherein the polynucleotide comprises a nucleic acid sequence as defined by SEQ ID NO. 6 or a nucleic acid sequence having at least 95% sequence identity to SEQ ID NO. 6.

13. The polynucleotide of claim 1, wherein at the polynucleotide fulfils least one of the following criteria:
the polynucleotide comprises a nucleic acid sequence as defined by SEQ ID NO. 8 or a nucleic acid sequence having at least 95% sequence identity to SEQ ID NO. 8,
the polynucleotide comprises a nucleic acid sequence as defined by SEQ ID NO. 10 or a nucleic acid sequence having at least 95% sequence identity to SEQ ID NO. 10,
the polynucleotide comprises a nucleic acid sequence as defined by SEQ ID NO. 15 or a nucleic acid sequence having at least 95% sequence identity to SEQ ID NO. 15,
the polynucleotide comprises a nucleic acid sequence as defined by SEQ ID NO. 16 or a nucleic acid sequence having at least 95% sequence identity to SEQ ID NO. 16, and
the polynucleotide comprises a nucleic acid sequence as defined by SEQ ID NO. 17 or a nucleic acid sequence having at least 95% sequence identity to SEQ ID NO. 17.

14. (canceled)

15. (canceled)

16. (canceled)

17. (canceled)

18. A live attenuated severe acute respiratory syndrome coronavirus 2 (SARS-CoV-2) comprising the polynucleotide according to claim 1.

19. The live attenuated SARS-CoV-2 according to claim 18, wherein
the SARS-CoV-2 has a nucleic acid sequence as defined by SEQ ID NO. 18 or a nucleic acid sequence having at least 98% sequence identity to SEQ ID NO. 18, or
the SARS-CoV-2 has a nucleic acid sequence as defined by SEQ ID NO. 19 or a nucleic acid sequence having at least 98% sequence identity to SEQ ID NO. 19.

20. (canceled)

21. A pharmaceutical composition comprising the live attenuated SARS-CoV-2 according to claim 18.

22. (canceled)

23. A method of vaccinating a human or animal patient, comprising the step of administering the pharmaceutical composition according to claim 21 to the patient.

24. The method according to claim 23, wherein the pharmaceutical composition is administered by an intranasal application, an oral application, or by parenteral administration.

25. The method according to claim 23, wherein a single dose of the pharmaceutical preparation comprises between 1*10³ and 1*10⁸ focus forming units of the live attenuated SARS-CoV-2.

26. The method according to claim 23, wherein the pharmaceutical preparation is administered to the patient at least two times, wherein a second administration is separated from a first administration by a first time period lying in range of from 2 weeks to 36 months.

27. (canceled)

28. (canceled)

29. (canceled)


SEQUENCE LISTING (19 sequences)

SEQ ID NO: 1 - RNA, 1482 nt, SARS-CoV-2
aaagauacuacugaagccuuugaaaaaaugguuucacuacuuucuguuuugcuuuccaugcagggugcuguagacauaaacaagcuuugugaagaaaugcuggacaacagggcaaccuuacaagcuauagccucagaguuuaguucccuuccaucauaugcagcuuuugcuacugcucaagaagcuuaugagcaggcuguugcuaauggugauucugaaguuguucuuaaaaaguugaagaagucuuugaauguggcuaaaucugaauuugaccgugaugcagccaugcaacguaaguuggaaaagauggcugaucaagcuaugacccaaauguauaaacaggcuagaucugaggacaagagggcaaaaguuacuagugcuaugcagacaaugcuuuucacuaugcuuagaaaguuggauaaugaugcacucaacaacauuaucaacaaugcaagagaugguuguguucccuugaacauaauaccucuuacaacagcagccaaacuaaugguugucauaccagacuauaacacauauaaaaauacgugugaugguacaacauuuacuuaugcaucagcauugugggaaauccaacagguuguagaugcagauaguaaaauuguucaacuuagugaaauuaguauggacaauucaccuaauuuagcauggccucuuauuguaacagcuuuaagggccaauucugcugucaaauuacagaauaaugagcuuaguccuguugcacuacgacagaugucuugugcugccgguacuacacaaacugcuugcacugaugacaaugcguuagcuuacuacaacacaacaaagggagguagguuuguacuugcacuguuauccgauuuacaggauuugaaaugggcuagauucccuaagagugauggaacugguacuaucuauacagaacuggaaccaccuuguagguuuguuacagacacaccuaaagguccuaaagugaaguauuuauacuuuauuaaaggauuaaacaaccuaaauagagguaugguacuugguaguuuagcugccacaguacgucuacaagcugguaaugcaacagaagugccugccaauucaacuguauuaucuuucugugcuuuugcuguagaugcugcuaaagcuuacaaagauuaucuagcuagugggggacaaccaaucacuaauuguguuaagauguuguguacacacacugguacuggucaggcaauaacaguuacaccggaagccaauauggaucaagaauccuuugguggugcaucguguugucuguacugccguugccacauagaucauccaaauccuaaaggauuuugugacuuaaaagguaaguauguacaaauaccuacaacuugugcuaaugacccuguggguuuuacacuuaaaaacacagucuguaccgucugcgguauguggaaagguuauggcuguaguugugaucaacuccgcgaacccaugcuucagucagcugau

SEQ ID NO: 2 - RNA, 1482 nt, SARS-CoV-2
aaagauacuaccgaagcauucgaaaaaaugguuagucuguuaucgguacuguuaucuaugcaaggugccguugacauuaauaaguuaugcgaagaaauguuagacaauagggcuacauugcaagcuaucgcuagugaauuuaguucacuaccaucauacgcugcauucgcuacagcucaagaggcauacgagcaggcagucgcuaacggugauuccgaaguagugcuuaaaaaacuuaaaaaaucacuuaacguugcgaaauccgaauuugauagggacgccgcuaugcaacguaaguuagagaaaauggccgaucaggcuaugacacaaauguauaaacaggcuagaucugaggauaaacgagcuaagguuacuagugcuaugcaaacuauguuguuuacuauguuacguaaacucgauaacgacgcacuuaacaauauaauuaauaacgcuagagacggaugcguaccacuuaauauuauaccguuaacuacugccgcuaaauugaugguaguuauaccugauuauaauacuuauaaaaauacaugugacgguacuacuuuuacauacgcuagugcauuaugggaaauacaacagguagucgacgccgauaguaagauagugcaauugucugagauaucuauggauaauaguccuaaucucgcauggccauuaaucguuaccgcauugcgugcuaacucugccguuaaguuacagaauaacgaauugucaccagucgcauugcgucaaaugucaugugccgcagguacgacacaaaccgcauguacagacgauaacgcauuagcuuauuauaauacuacuaagggagguagauucguacucgcacuauuauccgauuuacaggaucuuaaaugggcuagguuuccuaaaucugacgguacagguacuauauauaccgaacucgaaccuccauguagauucguuaccgauacaccuaagggaccuaagguuaaguaucuauauuuuauuaagggauugaauaaucuuaauagggguaugguauuagggucauuagccgcuacaguuagguugcaagccgguaacgcuaccgaagugccagcuaauaguacgguacuaucuuuuugugcauucgcaguugacgcugcuaaagcuuauaaagacuaucuagcuaguggaggucaaccuauuacuaauugcguuaaaauguuauguacacauacagguacaggucaagcuauuacgguuacaccugaagcuaauauggaucaggaaucauucggaggugcuaguuguugucuauauuguaggugucauaucgaucacccuaauccuaaggguuuuugcgaucuuaaggguaaguauguucagauaccuacuacaugugcuaacgaucccguagguuuuacacuuaaaaauacaguuuguacaguuugugguauguggaaagguuacgguuguucaugugaucaauuacgcgaaccuauguugcaauccgcugac

SEQ ID NO: 3 - RNA, 354 nt, SARS-CoV-2
uuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaagccuuacauuaagugggauuuguuaaaauaugacuucacggaagagagguuaaaacucuuugaccguuauuuuaaauau

SEQ ID NO: 4 - RNA, 354 nt, SARS-CoV-2
uuacgcguauacgcuaacuuaggcgaacgcguuagacaggcauuacuuaaaacagugcaauuuugugacgcuaugcguaacgccgguaucguagguguacuuacacuugauaaucaagaccuuaacgguaauugguacgauuuuggugauuuuauacagacuacaccugguucagguguacccguagucgauucuuauuauagucuguuaaugccuauacuuacacuuacacgugcauugacugccgaaucucacguugauacugaucugacuaagccuuauauuaaaugggaucuguuaaaauacgauuuuacagaggaacgauugaaauuguucgauagguauuuuaaguau

SEQ ID NO: 5 - RNA, 2339 nt, SARS-CoV-2
aaagauacuacugaagccuuugaaaaaaugguuucacuacuuucuguuuugcuuuccaugcagggugcuguagacauaaacaagcuuugugaagaaaugcuggacaacagggcaaccuuacaagcuauagccucagaguuuaguucccuuccaucauaugcagcuuuugcuacugcucaagaagcuuaugagcaggcuguugcuaauggugauucugaaguuguucuuaaaaaguugaagaagucuuugaauguggcuaaaucugaauuugaccgugaugcagccaugcaacguaaguuggaaaagauggcugaucaagcuaugacccaaauguauaaacaggcuagaucugaggacaagagggcaaaaguuacuagugcuaugcagacaaugcuuuucacuaugcuuagaaaguuggauaaugaugcacucaacaacauuaucaacaaugcaagagaugguuguguucccuugaacauaauaccucuuacaacagcagccaaacuaaugguugucauaccagacuauaacacauauaaaaauacgugugaugguacaacauuuacuuaugcaucagcauugugggaaauccaacagguuguagaugcagauaguaaaauuguucaacuuagugaaauuaguauggacaauucaccuaauuuagcauggccucuuauuguaacagcuuuaagggccaauucugcugucaaauuacagaauaaugagcuuaguccuguugcacuacgacagaugucuugugcugccgguacuacacaaacugcuugcacugaugacaaugcguuagcuuacuacaacacaacaaagggagguagguuuguacuugcacuguuauccgauuuacaggauuugaaaugggcuagauucccuaagagugauggaacugguacuaucuauacagaacuggaaccaccuuguagguuuguuacagacacaccuaaagguccuaaagugaaguauuuauacuuuauuaaaggauuaaacaaccuaaauagagguaugguacuugguaguuuagcugccacaguacgucuacaagcugguaaugcaacagaagugccugccaauucaacuguauuaucuuucugugcuuuugcuguagaugcugcuaaagcuuacaaagauuaucuagcuagugggggacaaccaaucacuaauuguguuaagauguuguguacacacacugguacuggucaggcaauaacaguuacaccggaagccaauauggaucaagaauccuuugguggugcaucguguugucuguacugccguugccacauagaucauccaaauccuaaaggauuuugugacuuaaaagguaaguauguacaaauaccuacaacuugugcuaaugacccuguggguuuuacacuuaaaaacacagucuguaccgucugcgguauguggaaagguuauggcuguaguugugaucaacuccgcgaacccaugcuucagucagcugaugcacaaucguuuuuaaacggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgccaacuuaggugaacguguacgccaagcuuuguuaaaaacaguacaauucugugaugccaugcgaaaugcugguauuguugguguacugacauuagauaaucaagaucucaaugguaacugguaugauuucggugauuucauacaaaccacgccagguaguggaguuccuguuguagauucuuauuauucauuguuaaugccuauauuaaccuugaccagggcuuuaacugcagagucacauguugacacugacuuaacaaagccuuacauuaagugggauuuguuaaaauaugacuucacggaagagagguuaaaacucuuugaccguuauuuuaaauau

SEQ ID NO: 6 - RNA, 2339 nt, SARS-CoV-2
aaagauacuaccgaagcauucgaaaaaaugguuagucuguuaucgguacuguuaucuaugcaaggugccguugacauuaauaaguuaugcgaagaaauguuagacaauagggcuacauugcaagcuaucgcuagugaauuuaguucacuaccaucauacgcugcauucgcuacagcucaagaggcauacgagcaggcagucgcuaacggugauuccgaaguagugcuuaaaaaacuuaaaaaaucacuuaacguugcgaaauccgaauuugauagggacgccgcuaugcaacguaaguuagagaaaauggccgaucaggcuaugacacaaauguauaaacaggcuagaucugaggauaaacgagcuaagguuacuagugcuaugcaaacuauguuguuuacuauguuacguaaacucgauaacgacgcacuuaacaauauaauuaauaacgcuagagacggaugcguaccacuuaauauuauaccguuaacuacugccgcuaaauugaugguaguuauaccugauuauaauacuuauaaaaauacaugugacgguacuacuuuuacauacgcuagugcauuaugggaaauacaacagguagucgacgccgauaguaagauagugcaauugucugagauaucuauggauaauaguccuaaucucgcauggccauuaaucguuaccgcauugcgugcuaacucugccguuaaguuacagaauaacgaauugucaccagucgcauugcgucaaaugucaugugccgcagguacgacacaaaccgcauguacagacgauaacgcauuagcuuauuauaauacuacuaagggagguagauucguacucgcacuauuauccgauuuacaggaucuuaaaugggcuagguuuccuaaaucugacgguacagguacuauauauaccgaacucgaaccuccauguagauucguuaccgauacaccuaagggaccuaagguuaaguaucuauauuuuauuaagggauugaauaaucuuaauagggguaugguauuagggucauuagccgcuacaguuagguugcaagccgguaacgcuaccgaagugccagcuaauaguacgguacuaucuuuuugugcauucgcaguugacgcugcuaaagcuuauaaagacuaucuagcuaguggaggucaaccuauuacuaauugcguuaaaauguuauguacacauacagguacaggucaagcuauuacgguuacaccugaagcuaauauggaucaggaaucauucggaggugcuaguuguugucuauauuguaggugucauaucgaucacccuaauccuaaggguuuuugcgaucuuaaggguaaguauguucagauaccuacuacaugugcuaacgaucccguagguuuuacacuuaaaaauacaguuuguacaguuugugguauguggaaagguuacgguuguucaugugaucaauuacgcgaaccuauguugcaauccgcugacgcacaaucguuuuuaaacggguuugcgguguaagugcagcccgucuuacaccgugcggcacaggcacuaguacugaugucguauacagggcuuuugacaucuacaaugauaaaguagcugguuuugcuaaauuccuaaaaacuaauuguugucgcuuccaagaaaaggacgaagaugacaauuuaauugauucuuacuuuguaguuaagagacacacuuucucuaacuaccaacaugaagaaacaauuuauaauuuacuuaaggauuguccagcuguugcuaaacaugacuucuuuaaguuuagaauagacggugacaugguaccacauauaucacgucaacgucuuacuaaauacacaauggcagaccucgucuaugcuuuaaggcauuuugaugaagguaauugugacacauuaaaagaaauacuugucacauacaauuguugugaugaugauuauuucaauaaaaaggacugguaugauuuuguagaaaacccagauauauuacgcguauacgcuaacuuaggcgaacgcguuagacaggcauuacuuaaaacagugcaauuuugugacgcuaugcguaacgccgguaucguagguguacuuacacuugauaaucaagaccuuaacgguaauugguacgauuuuggugauuuuauacagacuacaccugguucagguguacccguagucgauucuuauuauagucuguuaaugccuauacuuacacuuacacgugcauugacugccgaaucucacguugauacugaucugacuaagccuuauauuaaaugggaucuguuaaaauacgauuuuacagaggaacgauugaaauuguucgauagguauuuuaaguau

SEQ ID NO: 7 - RNA, 1146 nt, SARS-CoV-2
gguuuacaucuacugauuggacuagcuaaacguuuuaaggaaucaccuuuugaauuagaagauuuuauuccuauggacaguacaguuaaaaacuauuucauaacagaugcgcaaacagguucaucuaaguguguguguucuguuauugauuuauuacuugaugauuuuguugaaauaauaaaaucccaagauuuaucuguaguuucuaagguugucaaagugacuauugacuauacagaaauuucauuuaugcuuugguguaaagauggccauguagaaacauuuuacccaaaauuacaaucuagucaagcguggcaaccggguguugcuaugccuaaucuuuacaaaaugcaaagaaugcuauuagaaaagugugaccuucaaaauuauggugauagugcaacauuaccuaaaggcauaaugaugaaugucgcaaaauauacucaacugugucaauauuuaaacacauuaacauuagcuguacccuauaauaugagaguuauacauuuuggugcugguucugauaaaggaguugcaccagguacagcuguuuuaagacagugguugccuacggguacgcugcuugucgauucagaucuuaaugacuuugucucugaugcagauucaacuuugauuggugauugugcaacuguacauacagcuaauaaaugggaucucauuauuagugauauguacgacccuaagacuaaaaauguuacaaaagaaaaugacucuaaagaggguuuuuucacuuacauuuguggguuuauacaacaaaagcuagcucuuggagguuccguggcuauaaagauaacagaacauucuuggaaugcugaucuuuauaagcucaugggacacuucgcaugguggacagccuuuguuacuaaugugaaugcgucaucaucugaagcauuuuuaauuggauguaauuaucuuggcaaaccacgcgaacaaauagaugguuaugucaugcaugcaaauuacauauuuuggaggaauacaaauccaauucaguugucuuccuauucuuuauuugacaugaguaaauuuccccuuaaauuaagggguacugcuguuaugucuuuaaaagaaggucaaaucaaugauaugauuuuaucucuucuuaguaaagguagacuuauaauu

SEQ ID NO: 8 - RNA, 1146 nt, SARS-CoV-2
ggguuacauuuacuuauaggguuagcuaaaagauuuaaagaaucaccauucgaacucgaagacuuuauaccuauggauaguacgguuaagaauuauuuuauuacugacgcucaaaccgguucaucuaaaugcguuuguucaguuauagacuuacuguuagacgauuuugucgaaauuauuaagucucaggaucuaucagucguaucuaaagucguuaagguuacaaucgauuauacugagauaucuuuuauguuaugguguaaagacggucacguugagacuuuuuauccuaaauugcaaucuagucaagcuuggcaacccggugucgcuaugccuaaucuauauaaaaugcaacguauguuacucgaaaaaugcgauuuacagaauuacggugauuccgcuacauugccuaaagguauuaugaugaaugucgcuaaauauacacaauugugucaauaucuuaauacacuuacacuugcaguuccauauaauaugagagugauacauuuuggcgcaggaucugauaagggagucgcaccagguacugccguacuuagacaaugguuaccuacagguacacuguuagucgauuccgaucuuaacgauuuuguuucugacgcugauucuacacuuauaggcgauugugcuacagugcauaccgcuaauaaaugggaucuuauuauauccgauauguacgauccuaaaacuaaaaacguuacuaaggaaaacgauucuaaagaggguuuuuuuacuuauauuugugguuuuauacagcaaaaauuagcguuaggcggauccguugcgauuaagauuaccgaacauaguuggaaugcugaucuauauaagcuuaugggucauuucgcaugguggacugcauucguuacgaauguuaacgcuucuaguuccgaagcuuuuuuaaucggauguaauuauuuagguaagccuagggaacagauugacggauacguuaugcaugcuaauuauauuuuuuggcguaauacuaauccuauucaauugucuaguuauucauuauucgauaugucuaaauuuccacuuaaacuuagggguacugccguuaugucacuuaaggaaggucaaauuaacgauaugauacuaucauuguuaucuaaagguagauugauuaua

SEQ ID NO: 9 - RNA, 999 nt, SARS-CoV-2
aaccaauuuaauagugcuauuggcaaaauucaagacucacuuucuuccacagcaagugcacuuggaaaacuucaagauguggucaaccaaaaugcacaagcuuuaaacacgcuuguuaaacaacuuagcuccaauuuuggugcaauuucaaguguuuuaaaugauauccuuucacgucuugacaaaguugaggcugaagugcaaauugauagguugaucacaggcagacuucaaaguuugcagacauaugugacucaacaauuaauuagagcugcagaaaucagagcuucugcuaaucuugcugcuacuaaaaugucagaguguguacuuggacaaucaaaaagaguugauuuuuguggaaagggcuaucaucuuauguccuucccucagucagcaccucaugguguagucuucuugcaugugacuuaugucccugcacaagaaaagaacuucacaacugcuccugccauuugucaugauggaaaagcacacuuuccucgugaaggugucuuuguuucaaauggcacacacugguuuguaacacaaaggaauuuuuaugaaccacaaaucauuacuacagacaacacauuugugucugguaacugugauguuguaauaggaauugucaacaacacaguuuaugauccuuugcaaccugaauuagacucauucaaggaggaguuagauaaauauuuuaagaaucauacaucaccagauguugauuuaggugacaucucuggcauuaaugcuucaguuguaaacauucaaaaagaaauugaccgccucaaugagguugccaagaauuuaaaugaaucucucaucgaucuccaagaacuuggaaaguaugagcaguauauaaaauggccaugguacauuuggcuagguuuuauagcuggcuugauugccauaguaauggugacaauuaugcuuugcuguaugaccaguugcuguaguugucucaagggcuguuguucuuguggauccugcugcaaauuugau

SEQ ID NO: 10 - RNA, 999 nt, SARS-CoV-2
aaccaauuuaauuccgcuauagguaagauucaagacucauugucuaguaccgcuagugcauuagguaaguugcaagacgucguuaaccaaaacgcucaagcacuuaauacacuuguuaagcaauugucuaguaauuuuggcgcuauuaguucagugcuuaacgauauucuaucacgucuugauaaagucgaagccgaagugcaaaucgauagauugauuaccgguagauugcaaucucuucaaacuuauguuacacaacaauugauuagggcugccgaaauuagggcuagugcuaaucucgcagcuacuaaaaugucugaaugcguacucggucaaucuaaacgugucgauuuuugcgguaagggauaucaucuuaugucuuuuccacaauccgcaccacauggagugguuuuuuuacacguuacauacguuccagcucaggaaaaaaauuuuacuaccgcaccagcuauuugucaugacgguaaggcacauuuuccuagagagggaguauucguuucuaacgguacacauugguucguuacacaacguaauuuuuacgagccacaaauuauuacuacugauaauacauucguuagcgguaauugcgacguagugauagguauaguuaauaauacaguuuacgauccauugcaaccugaacucgauucuuuuaaagaggaacucgauaaguauuuuaaaaaccauacaucaccugacguugacuuaggcgauauuuccgguauuaacgcuagcguaguuaauauucaaaaagaaauugauagacuuaacgaagucgcuaaaaaccuuaacgaaucacuuaucgaucuucaagaguuagguaaguaugagcaauauauuaaauggccuugguauauuugguuaggguuuauagccggucuuaucgcaaucguuaugguuacaauuauguuauguuguaugacaucauguuguucaugucuuaagggauguuguucaugcggaucauguuguaaauuugac

SEQ ID NO: 11 - protein, 494 aa, SARS-CoV-2
KDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSAD

SEQ ID NO: 12 - protein, 118 aa, SARS-CoV-2
LRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKY

SEQ ID NO: 13 - protein, 382 aa, SARS-CoV-2
GLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLII

SEQ ID NO: 14 - protein, 333 aa, SARS-CoV-2
NQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFD

SEQ ID NO: 15 - DNA, 2943 bp, SARS-CoV-2
tcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactaccgaagcattcgaaaaaatggttagtctgttatcggtactgttatctatgcaaggtgccgttgacattaataagttatgcgaagaaatgttagacaatagggctacattgcaagctatcgctagtgaatttagttcactaccatcatacgctgcattcgctacagctcaagaggcatacgagcaggcagtcgctaacggtgattccgaagtagtgcttaaaaaacttaaaaaatcacttaacgttgcgaaatccgaatttgatagggacgccgctatgcaacgtaagttagagaaaatggccgatcaggctatgacacaaatgtataaacaggctagatctgaggataaacgagctaaggttactagtgctatgcaaactatgttgtttactatgttacgtaaactcgataacgacgcacttaacaatataattaataacgctagagacggatgcgtaccacttaatattataccgttaactactgccgctaaattgatggtagttatacctgattataatacttataaaaatacatgtgacggtactacttttacatacgctagtgcattatgggaaatacaacaggtagtcgacgccgatagtaagatagtgcaattgtctgagatatctatggataatagtcctaatctcgcatggccattaatcgttaccgcattgcgtgctaactctgccgttaagttacagaataacgaattgtcaccagtcgcattgcgtcaaatgtcatgtgccgcaggtacgacacaaaccgcatgtacagacgataacgcattagcttattataatactactaagggaggtagattcgtactcgcactattatccgatttacaggatcttaaatgggctaggtttcctaaatctgacggtacaggtactatatataccgaactcgaacctccatgtagattcgttaccgatacacctaagggacctaaggttaagtatctatattttattaagggattgaataatcttaataggggtatggtattagggtcattagccgctacagttaggttgcaagccggtaacgctaccgaagtgccagctaatagtacggtactatctttttgtgcattcgcagttgacgctgctaaagcttataaagactatctagctagtggaggtcaacctattactaattgcgttaaaatgttatgtacacatacaggtacaggtcaagctattacggttacacctgaagctaatatggatcaggaatcattcggaggtgctagttgttgtctatattgtaggtgtcatatcgatcaccctaatcctaagggtttttgcgatcttaagggtaagtatgttcagatacctactacatgtgctaacgatcccgtaggttttacacttaaaaatacagtttgtacagtttgtggtatgtggaaaggttacggttgttcatgtgatcaattacgcgaacctatgttgcaatccgctgacgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgctaacttaggcgaacgcgttagacaggcattacttaaaacagtgcaattttgtgacgctatgcgtaacgccggtatcgtaggtgtacttacacttgataatcaagaccttaacggtaattggtacgattttggtgattttatacagactacacctggttcaggtgtacccgtagtcgattcttattatagtctgttaatgcctatacttacacttacacgtgcattgactgccgaatctcacgttgatactgatctgactaagccttatattaaatgggatctgttaaaatacgattttacagaggaacgattgaaattgttcgataggtattttaagtattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattact

SEQ ID NO: 16 - DNA, 3178 bp, SARS-CoV-2
tggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtgggttacatttacttatagggttagctaaaagatttaaagaatcaccattcgaactcgaagactttatacctatggatagtacggttaagaattattttattactgacgctcaaaccggttcatctaaatgcgtttgttcagttatagacttactgttagacgattttgtcgaaattattaagtctcaggatctatcagtcgtatctaaagtcgttaaggttacaatcgattatactgagatatcttttatgttatggtgtaaagacggtcacgttgagactttttatcctaaattgcaatctagtcaagcttggcaacccggtgtcgctatgcctaatctatataaaatgcaacgtatgttactcgaaaaatgcgatttacagaattacggtgattccgctacattgcctaaaggtattatgatgaatgtcgctaaatatacacaattgtgtcaatatcttaatacacttacacttgcagttccatataatatgagagtgatacattttggcgcaggatctgataagggagtcgcaccaggtactgccgtacttagacaatggttacctacaggtacactgttagtcgattccgatcttaacgattttgtttctgacgctgattctacacttataggcgattgtgctacagtgcataccgctaataaatgggatcttattatatccgatatgtacgatcctaaaactaaaaacgttactaaggaaaacgattctaaagagggtttttttacttatatttgtggttttatacagcaaaaattagcgttaggcggatccgttgcgattaagattaccgaacatagttggaatgctgatctatataagcttatgggtcatttcgcatggtggactgcattcgttacgaatgttaacgcttctagttccgaagcttttttaatcggatgtaattatttaggtaagcctagggaacagattgacggatacgttatgcatgctaattatattttttggcgtaatactaatcctattcaattgtctagttattcattattcgatatgtctaaatttccacttaaacttaggggtactgccgttatgtcacttaaggaaggtcaaattaacgatatgatactatcattgttatctaaaggtagattgattataagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgc

SEQ ID NO: 17 - DNA, 2966 bp, SARS-CoV-2
aatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaattccgctataggtaagattcaagactcattgtctagtaccgctagtgcattaggtaagttgcaagacgtcgttaaccaaaacgctcaagcacttaatacacttgttaagcaattgtctagtaattttggcgctattagttcagtgcttaacgatattctatcacgtcttgataaagtcgaagccgaagtgcaaatcgatagattgattaccggtagattgcaatctcttcaaacttatgttacacaacaattgattagggctgccgaaattagggctagtgctaatctcgcagctactaaaatgtctgaatgcgtactcggtcaatctaaacgtgtcgatttttgcggtaagggatatcatcttatgtcttttccacaatccgcaccacatggagtggtttttttacacgttacatacgttccagctcaggaaaaaaattttactaccgcaccagctatttgtcatgacggtaaggcacattttcctagagagggagtattcgtttctaacggtacacattggttcgttacacaacgtaatttttacgagccacaaattattactactgataatacattcgttagcggtaattgcgacgtagtgataggtatagttaataatacagtttacgatccattgcaacctgaactcgattcttttaaagaggaactcgataagtattttaaaaaccatacatcacctgacgttgacttaggcgatatttccggtattaacgctagcgtagttaatattcaaaaagaaattgatagacttaacgaagtcgctaaaaaccttaacgaatcacttatcgatcttcaagagttaggtaagtatgagcaatatattaaatggccttggtatatttggttagggtttatagccggtcttatcgcaatcgttatggttacaattatgttatgttgtatgacatcatgttgttcatgtcttaagggatgttgttcatgcggatcatgttgtaaatttgacgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatga

SEQ ID NO: 18 - DNA, 29899 bp, SARS-CoV-2
atattaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtgggttacatttacttatagggttagctaaaagatttaaagaatcaccattcgaactcgaagactttatacctatggatagtacggttaagaattattttattactgacgctcaaaccggttcatctaaatgcgtttgttcagttatagacttactgttagacgattttgtcgaaattattaagtctcaggatctatcagtcgtatctaaagtcgttaaggttacaatcgattatactgagatatcttttatgttatggtgtaaagacggtcacgttgagactttttatcctaaattgcaatctagtcaagcttggcaacccggtgtcgctatgcctaatctatataaaatgcaacgtatgttactcgaaaaatgcgatttacagaattacggtgattccgctacattgcctaaaggtattatgatgaatgtcgctaaatatacacaattgtgtcaatatcttaatacacttacacttgcagttccatataatatgagagtgatacattttggcgcaggatctgataagggagtcgcaccaggtactgccgtacttagacaatggttacctacaggtacactgttagtcgattccgatcttaacgattttgtttctgacgctgattctacacttataggcgattgtgctacagtgcataccgctaataaatgggatcttattatatccgatatgtacgatcctaaaactaaaaacgttactaaggaaaacgattctaaagagggtttttttacttatatttgtggttttatacagcaaaaattagcgttaggcggatccgttgcgattaagattaccgaacatagttggaatgctgatctatataagcttatgggtcatttcgcatggtggactgcattcgttacgaatgttaacgcttctagttccgaagcttttttaatcggatgtaattatttaggtaagcctagggaacagattgacggatacgttatgcatgctaattatattttttggcgtaatactaatcctattcaattgtctagttattcattattcgatatgtctaaatttccacttaaacttaggggtactgccgttatgtcacttaaggaaggtcaaattaacgatatgatactatcattgttatctaaaggtagattgattataagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 19 - DNA, 29899 bp, SARS-CoV-2
atattaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaattccgctataggtaagattcaagactcattgtctagtaccgctagtgcattaggtaagttgcaagacgtcgttaaccaaaacgctcaagcacttaatacacttgttaagcaattgtctagtaattttggcgctattagttcagtgcttaacgatattctatcacgtcttgataaagtcgaagccgaagtgcaaatcgatagattgattaccggtagattgcaatctcttcaaacttatgttacacaacaattgattagggctgccgaaattagggctagtgctaatctcgcagctactaaaatgtctgaatgcgtactcggtcaatctaaacgtgtcgatttttgcggtaagggatatcatcttatgtcttttccacaatccgcaccacatggagtggtttttttacacgttacatacgttccagctcaggaaaaaaattttactaccgcaccagctatttgtcatgacggtaaggcacattttcctagagagggagtattcgtttctaacggtacacattggttcgttacacaacgtaatttttacgagccacaaattattactactgataatacattcgttagcggtaattgcgacgtagtgataggtatagttaataatacagtttacgatccattgcaacctgaactcgattcttttaaagaggaactcgataagtattttaaaaaccatacatcacctgacgttgacttaggcgatatttccggtattaacgctagcgtagttaatattcaaaaagaaattgatagacttaacgaagtcgctaaaaaccttaacgaatcacttatcgatcttcaagagttaggtaagtatgagcaatatattaaatggccttggtatatttggttagggtttatagccggtcttatcgcaatcgttatggttacaattatgttatgttgtatgacatcatgttgttcatgtcttaagggatgttgttcatgcggatcatgttgtaaatttgacgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa